SEQUENCE LISTING (79 sequences)

SEQ ID NO: 1 - DNA, 1680 bp, K. pneumoniae
atggacaaacagtatccggtacgccagtgggcgcacggcgccgatctcgtcgtcagtcagctggaagctcagggagtacgccaggtgttcggcatccccggcgccaaaatcgacaaggtctttgattcactgctggattcctccattcgcattattccggtacgccacgaagccaacgccgcatttatggccgccgccgtcggacgcattaccggcaaagcgggcgtggcgctggtcacctccggtccgggctgttccaacctgatcaccggcatggccaccgcgaacagcgaaggcgacccggtggtggccctgggcggcgcggtaaaacgcgccgataaagcgaagcaggtccaccagagtatggatacggtggcgatgttcagcccggtcaccaaatacgccatcgaggtgacggcgccggatgcgctggcggaagtggtctccaacgccttccgcgccgccgagcagggccggccgggcagcgcgttcgttagcctgccgcaggatgtggtcgatggcccggtcagcggcaaagtgctgccggccagcggggccccgcagatgggcgccgcgccggatgatgccatcgaccaggtggcgaagcttatcgcccaggcgaagaacccgatcttcctgctcggcctgatggccagccagccggaaaacagcaaggcgctgcgccgtttgctggagaccagccatattccagtcaccagcacctatcaggccgccggagcggtgaatcaggataacttctctcgcttcgccggccgggttgggctgtttaacaaccaggccggggaccgtctgctgcagctcgccgacctggtgatctgcatcggctacagcccggtggaatacgaaccggcgatgtggaacagcggcaacgcgacgctggtgcacatcgacgtgctgcccgcctatgaagagcgcaactacaccccggatgtcgagctggtgggcgatatcgccggcactctcaacaagctggcgcaaaatatcgatcatcggctggtgctctccccgcaggcggcggagatcctccgcgaccgccagcaccagcgcgagctgctggaccgccgcggcgcgcagctcaaccagtttgccctgcatcccctgcgcatcgttcgcgccatgcaggatatcgtcaacagcgacgtcacgttgaccgtggacatgggcagcttccatatctggattgcccgctacctgtacacgttccgcgcccgtcaggtgatgatctccaacggccagcagaccatgggcgtcgccctgccctgggctatcggcgcctggctggtcaatcctgagcgcaaagtggtctccgtctccggcgacggcggcttcctgcagtcgagcatggagctggagaccgccgtccgcctgaaagccaacgtgctgcatcttatctgggtcgataacggctacaacatggtcgctatccaggaagagaaaaaatatcagcgcctgtccggcgtcgagtttgggccgatggattttaaagcctatgccgaatccttcggcgcgaaagggtttgccgtggaaagcgccgaggcgctggagccgaccctgcgcgcggcgatggacgtcgacggcccggcggtagtggccatcccggtggattatcgcgataacccgctgctgatgggccagctgcatctgagtcagattctgtaa

SEQ ID NO: 2 - protein, 559 aa, K. pneumoniae
MDKQYPVRQWAHGADLVVSQLEAQGVRQVFGIPGAKIDKVFDSLLDSSIRIIPVRHEANAAFMAAAVGRITGKAGVALVTSGPGCSNLITGMATANSEGDPVVALGGAVKRADKAKQVHQSMDTVAMFSPVTKYAIEVTAPDALAEVVSNAFRAAEQGRPGSAFVSLPQDVVDGPVSGKVLPASGAPQMGAAPDDAIDQVAKLIAQAKNPIFLLGLMASQPENSKALRRLLETSHIPVTSTYQAAGAVNQDNFSRFAGRVGLFNNQAGDRLLQLADLVICIGYSPVEYEPAMWNSGNATLVHIDVLPAYEERNYTPDVELVGDIAGTLNKLAQNIDHRLVLSPQAAEILRDRQHQRELLDRRGAQLNQFALHPLRIVRAMQDIVNSDVTLTVDMGSFHIWIARYLYTFRARQVMISNGQQTMGVALPWAIGAWLVNPERKVVSVSGDGGFLQSSMELETAVRLKANVLHLIWVDNGYNMVAIQEEKKYQRLSGVEFGPMDFKAYAESFGAKGFAVESAEALEPTLRAAMDVDGPAVVAIPVDYRDNPLLMGQLHLSQIL

SEQ ID NO: 3 - DNA, 1716 bp, Bacillus subtilis
atgttgacaaaagcaacaaaagaacaaaaatcccttgtgaaaaacagaggggcggagcttgttgttgattgcttagtggagcaaggtgtcacacatgtatttggcattccaggtgcaaaaattgatgcggtatttgacgctttacaagataaaggacctgaaattatcgttgcccggcacgaacaaaacgcagcattcatggcccaagcagtcggccgtttaactggaaaaccgggagtcgtgttagtcacatcaggaccgggtgcctctaacttggcaacaggcctgctgacagcgaacactgaaggagaccctgtcgttgcgcttgctggaaacgtgatccgtgcagatcgtttaaaacggacacatcaatctttggataatgcggcgctattccagccgattacaaaatacagtgtagaagttcaagatgtaaaaaatataccggaagctgttacaaatgcatttaggatagcgtcagcagggcaggctggggccgcttttgtgagctttccgcaagatgttgtgaatgaagtcacaaatacgaaaaacgtgcgtgctgttgcagcgccaaaactcggtcctgcagcagatgatgcaatcagtgcggccatagcaaaaatccaaacagcaaaacttcctgtcgttttggtcggcatgaaaggcggaagaccggaagcaattaaagcggttcgcaagcttttgaaaaaggttcagcttccatttgttgaaacatatcaagctgccggtaccctttctagagatttagaggatcaatattttggccgtatcggtttgttccgcaaccagcctggcgatttactgctagagcaggcagatgttgttctgacgatcggctatgacccgattgaatatgatccgaaattctggaatatcaatggagaccggacaattatccatttagacgagattatcgctgacattgatcatgcttaccagcctgatcttgaattgatcggtgacattccgtccacgatcaatcatatcgaacacgatgctgtgaaagtggaatttgcagagcgtgagcagaaaatcctttctgatttaaaacaatatatgcatgaaggtgagcaggtgcctgcagattggaaatcagacagagcgcaccctcttgaaatcgttaaagagttgcgtaatgcagtcgatgatcatgttacagtaacttgcgatatcggttcgcacgccatttggatgtcacgttatttccgcagctacgagccgttaacattaatgatcagtaacggtatgcaaacactcggcgttgcgcttccttgggcaatcggcgcttcattggtgaaaccgggagaaaaagtggtttctgtctctggtgacggcggtttcttattctcagcaatggaattagagacagcagttcgactaaaagcaccaattgtacacattgtatggaacgacagcacatatgacatggttgcattccagcaattgaaaaaatataaccgtacatctgcggtcgatttcggaaatatcgatatcgtgaaatatgcggaaagcttcggagcaactggcttgcgcgtagaatcaccagaccagctggcagatgttctgcgtcaaggcatgaacgctgaaggtcctgtcatcatcgatgtcccggttgactacagtgataacattaatttagcaagtgacaagcttccgaaagaattcggggaactcatgaaaacgaaagctctctag

SEQ ID NO: 4 - protein, 571 aa, Bacillus subtilis
MLTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 5 - DNA, 1665 bp, Lactococcus lactis
atgtctgagaaacaatttggggcgaacttggttgtcgatagtttgattaaccataaagtgaagtatgtatttgggattccaggagcaaaaattgaccgggtttttgatttattagaaaatgaagaaggccctcaaatggtcgtgactcgtcatgagcaaggagctgctttcatggctcaagctgtcggtcgtttaactggcgaacctggtgtagtagttgttacgagtgggcctggtgtatcaaaccttgcgactccgcttttgaccgcgacatcagaaggtgatgctattttggctatcggtggacaagttaaacgaagtgaccgtcttaaacgtgcgcaccaatcaatggataatgctggaatgatgcaatcagcaacaaaatattcagcagaagttcttgaccctaatacactttctgaatcaattgccaacgcttatcgtattgcaaaatcaggacatccaggtgcaactttcttatcaatcccccaagatgtaacggatgccgaagtatcaatcaaagccattcaaccactttcagaccctaaaatggggaatgcctctattgatgacattaattatttagcacaagcaattaaaaatgctgtattgccagtaattttggttggagctggtgcttcagatgctaaagtcgcttcatccttgcgtaatctattgactcatgttaatattcctgtcgttgaaacattccaaggtgcaggggttatttcacatgatttagaacatactttttatggacgtatcggtcttttccgcaatcaaccaggcgatatgcttctgaaacgttctgaccttgttattgctgttggttatgacccaattgaatatgaagctcgtaactggaatgcagaaattgatagtcgaattatcgttattgataatgccattgctgaaattgatacttactaccaaccagagcgtgaattaattggtgatatcgcagcaacattggataatcttttaccagctgttcgtggctacaaaattccaaaaggaacaaaagattatctcgatggccttcatgaagttgctgagcaacacgaatttgatactgaaaatactgaagaaggtagaatgcaccctcttgatttggtcagcactttccaagaaatcgtcaaggatgatgaaacagtaaccgttgacgtaggttcactctacatttggatggcacgtcatttcaaatcatacgaaccacgtcatctcctcttctcaaacggaatgcaaacactcggagttgcacttccttgggcaattacagccgcattgttgcgcccaggtaaaaaagtttattcacactctggtgatggaggcttccttttcacagggcaagaattggaaacagctgtacgtttgaatcttccaatcgttcaaattatctggaatgacggccattatgatatggttaaattccaagaagaaatgaaatatggtcgttcagcagccgttgattttggctatgttgattacgtaaaatatgctgaagcaatgagagcaaaaggttaccgtgcacacagcaaagaagaacttgctgaaattctcaaatcaatcccagatactactggaccggtggtaattgacgttcctttggactattctgataacattaaattagcagaaaaattattgcctgaagagttttattga

SEQ ID NO: 6 - protein, 554 aa, Lactococcus lactis
MSEKQFGANLVVDSLINHKVKYVFGIPGAKIDRVFDLLENEEGPQMVVTRHEQGAAFMAQAVGRLTGEPGVVVVTSGPGVSNLATPLLTATSEGDAILAIGGQVKRSDRLKRAHQSMDNAGMMQSATKYSAEVLDPNTLSESIANAYRIAKSGHPGATFLSIPQDVTDAEVSIKAIQPLSDPKMGNASIDDINYLAQAIKNAVLPVILVGAGASDAKVASSLRNLLTHVNIPVVETFQGAGVISHDLEHTFYGRIGLFRNQPGDMLLKRSDLVIAVGYDPIEYEARNWNAEIDSRIIVIDNAIAEIDTYYQPERELIGDIAATLDNLLPAVRGYKIPKGTKDYLDGLHEVAEQHEFDTENTEEGRMHPLDLVSTFQEIVKDDETVTVDVGSLYIWMARHFKSYEPRHLLFSNGMQTLGVALPWAITAALLRPGKKVYSHSGDGGFLFTGQELETAVRLNLPIVQIIWNDGHYDMVKFQEEMKYGRSAAVDFGYVDYVKYAEAMRAKGYRAHSKEELAEILKSIPDTTGPVVIDVPLDYSDNIKLAEKLLPEEFY

SEQ ID NO: 7 - DNA, 1665 bp, Staphylococcus aureus
  CDS (1)..(1665)
atgactgataaaaagtacactgcagccgatatggttattgatactttgaaaaataatggggtagaatatgtttttggtattccgggtgcaaagatagactatctatttaatgctttaattgatgatggtcctgaacttattgtcactcgtcatgaacaaaatgctgcaatgatggcacaaggtattggaagattaacaggtaaaccgggtgtagtacttgttacaagtggccctggtgtaagtaatttaacgactggactattaacagctacatctgaaggggatcctgtattagcgttaggtggccaagtgaaacgtaatgatttattacgattaacgcatcaaagtattgataatgctgcgctattaaaatattcatcaaaatacagtgaagaagtacaagatcctgaatcattatcagaagttatgacaaatgcaattcgaattgctacttcaggaaaaaatggcgcaagttttattagtattccgcaagacgttatttcttcaccagttgaatctaaagctatatcactttgccaaaaaccaaatttaggagtaccgagtgaacaagatattaatgatgtcattgaagcgattaaaaatgcatcatttcctgttttattagctggtatgagaagttcaagtgcagaagaaacaaatgccattcgcaaattagttgagcgcacgaatttaccagttgtagaaacattccaaggtgcaggtgtaattagtcgtgaattagaaaatcatttcttcggtcgtgtgggcttattccgcaatcaagttggtgatgaattattacgtaaaagtgatttagttgttacaatcggttatgatccaattgaatacgaagctagtaactggaataaagaattagaaacacaaattatcaatattgacgaagttcaagctgaaattactaattatatgcaaccgaaaaaagagttgattggtaatattgctaaaacgattgaaatgatttctgaaaaagtggatgagccatttataaatcaacaacatttagacgaattagaacaattaagaacacatattgatgaagaaactggtattaaagcgacgcatgaagaaggaattctacatccagtggaaattattgaatctatgcaaaaggtattaactgatgatactactgtaacagttgatgttggaagtcactatatttggatggcacgtaatttcagaagttacaatccaagacatttattatttagcaatggtatgcaaacgcttggtgtagcattaccgtgggcaatttcagctgcacttgtgcgccctaatacgcaagttgtgtccgttgctggcgatggtggctttttattttcatcacaagatttagaaacggccgtacgtaaaaatttaaatatcatccagcttatttggaatgatggaaaatataacatggttgaattccaagaagaaatgaaatataaacgttcgtcaggtgtagacttcggtcctgtagattttgtaaaatatgcagaatcatttggcgcgaaaggtttacgagttactaatcaagaagaattagaagcggcaattaaagagggctatgaaacagatggtccagtattaattgatatacctgtaaattacaaagataatatcaaactttcaacaaatatgttacctgacgtatttaactaa

SEQ ID NO: 8 - protein, 554 aa, Staphylococcus aureus
MTDKKYTAADMVIDTLKNNGVEYVFGIPGAKIDYLFNALIDDGPELIVTRHEQNAAMMAQGIGRLTGKPGVVLVTSGPGVSNLTTGLLTATSEGDPVLALGGQVKRNDLLRLTHQSIDNAALLKYSSKYSEEVQDPESLSEVMTNAIRIATSGKNGASFISIPQDVISSPVESKAISLCQKPNLGVPSEQDINDVIEAIKNASFPVLLAGMRSSSAEETNAIRKLVERTNLPVVETFQGAGVISRELENHFFGRVGLFRNQVGDELLRKSDLVVTIGYDPIEYEASNWNKELETQIINIDEVQAEITNYMQPKKELIGNIAKTIEMISEKVDEPFINQQHLDELEQLRTHIDEETGIKATHEEGILHPVEIIESMQKVLTDDTTVTVDVGSHYIWMARNFRSYNPRHLLFSNGMQTLGVALPWAISAALVRPNTQVVSVAGDGGFLFSSQDLETAVRKNLNIIQLIWNDGKYNMVEFQEEMKYKRSSGVDFGPVDFVKYAESFGAKGLRVTNQEELEAAIKEGYETDGPVLIDIPVNYKDNIKLSTNMLPDVFN

SEQ ID NO: 9 - DNA, 1698 bp, Listeria monocytogenes
  CDS (1)..(1698)
atggcgaaactagaaaaagaccaagaaaaagtaataacacaagggaaatcaggagcggatttagttgtagacagcttaattaatcaaggtgttacgcatgtattcgggattccgggagcgaaaattgataaagtttttgatgtgatggaagaacgtggaccagaattaattgtcagtcgtcatgaacaaaatgcggcgtttatggctgctgctatcggtcgtctaaccgggaaacctggtgttgtacttgtaactagtggacctggcgcatcgaatcttgcaacagggcttgtaaccgcaactgcagaaggagatccagtcgttgcgattgctggtaacgtaacaaggcaagaccgcttaaaaagaacccaccaatcaatggataatgcagcacttttccgtccgattacaaaatacagcgaagaagtagttcacgccgaaagtattccagaagcaatcactaacgcttttcgctcggcaacagaaccaaaccaaggcgctgcttttgtcagtttgccacaagatatcgtgaacgaaccaaacgtaccagtaaaagcgattcgcccacttgctaaaccagaaaatggtcctgcttccaaagaacaagttgcaaaacttgttacacgtttgaaaaaagcgaaattaccggtattgctattgggtatgcgagcatctagtccagaagtaactggtgcaattcgtcgcttactccaaaaaacaagtatcccagtagtagaaactttccaagcagctggcgtcatttcacgcgacttagaagataacttctttggacgtgttggtctgttccgcaaccaaccaggggatattttgttaaataaagctgatttagttattacagtgggttatgatccaattgaatacgatccaaaagcttggaatgcctctggtgatagaacgattgtccatttagacgacattcgcgctgatattgatcattattaccaaccagtgacagagctagtcggaaacatcgcgcttactttagaccgagtgaatgcgaaattcagcggtttagaattagcggaaaaagaacttgaaacattaaaagaacttcatgctcaattagaagagcgagatgttccgccagaaagtgatgaaactaaccgagtacatccattgtcggtcattcaaacactacgttcggcaattgatgacaacgtaactgtgacagtcgacgttggttcacattatatttggatggcacgtcatttccgctcctatgaaccacgccgtctgcttttcagtaacggtatgcaaacgcttggtgttgcgcttccttggggaattgctgcaacacttgtacatccgggtgaaaaagtggtttcgatttctggtgacggtggtttcttattttccgcgatggaattagaaacagctgtccgcttgcgtgcgccacttgtacacctagtatggaatgacggaagctatgacatggttgctttccaacaaaaaatgaaatacggcaaagaagcagctgttcgttttggcgatgttgatatcgtaaaatttgcagaaagtttcggagcaaaaggtcttcgcgtaacaaatccagcagaactttctgatgtgttaaaagaagcgcttgaaacagaaggacccgtcgttgtagatattccaattgattaccgtgataacatcaaacttggcgaaactttactacctgaccaattttattaa

SEQ ID NO: 10 - protein, 565 aa, Listeria monocytogenes
MAKLEKDQEKVITQGKSGADLVVDSLINQGVTHVFGIPGAKIDKVFDVMEERGPELIVSRHEQNAAFMAAAIGRLTGKPGVVLVTSGPGASNLATGLVTATAEGDPVVAIAGNVTRQDRLKRTHQSMDNAALFRPITKYSEEVVHAESIPEAITNAFRSATEPNQGAAFVSLPQDIVNEPNVPVKAIRPLAKPENGPASKEQVAKLVTRLKKAKLPVLLLGMRASSPEVTGAIRRLLQKTSIPVVETFQAAGVISRDLEDNFFGRVGLFRNQPGDILLNKADLVITVGYDPIEYDPKAWNASGDRTIVHLDDIRADIDHYYQPVTELVGNIALTLDRVNAKFSGLELAEKELETLKELHAQLEERDVPPESDETNRVHPLSVIQTLRSAIDDNVTVTVDVGSHYIWMARHFRSYEPRRLLFSNGMQTLGVALPWGIAATLVHPGEKVVSISGDGGFLFSAMELETAVRLRAPLVHLVWNDGSYDMVAFQQKMKYGKEAAVRFGDVDIVKFAESFGAKGLRVTNPAELSDVLKEALETEGPVVVDIPIDYRDNIKLGETLLPDQFY

SEQ ID NO: 11 - DNA, 1680 bp, Streptococcus mutans
  CDS (1)..(1680)
atgaccgaaataaataaggaaggctatggggctgacctgattgtagacagcctcattaatcatgatgtcaactatgtttttggaatccctggtgcaaaaattgatcgtgtctttgataccttagaagataaggggccagaacttattgtagcacgccatgagcaaaatgctgcttttatggctcaaggaattggccgtattactggtgagcctggtgttgtgattacaaccagcggtcccggtgtttccaatctggtgactggtcttgttactgcgacagctgagggagatcctgtccttgctattggtggtcaggttaaacgtgctgatttgctcaaacgggctcaccagtcaatgaataatgttgctatgctcgatcccattaccaaatattcagcagaaattcaggatcccgcaacactttcagaaaatattgctaatgcctatcgtttggctaaagcaggaaagccgggagctagtttcttatctattcctcaagatataactgatagtcctgttactgtcaaggcgattaagcccttgacagatcctaaactaggttcagcgtcagttgctgatattaattatttggcacaggccataaaaaatgcggtccttcctgtcttacttttaggaaatggtgcgtcaacggctgcagttacagcttctattcgccgtttgttaggagctgtcaagctgccagtcgttgaaactttccaaggagctggtattgtttcaagagatttagaagaggacactttttttggtcgtgtggggctttttcgtaatcagcccggagatatgttgctgaagcgttctgacttagttatcgctattggctatgatcctattgaatatgaagcgcgcaattggaatgctgaaatttcggctcgcattatcgttattgatgttgctccagctgaaattgatacttatttccaacctgaacgtgaattaattggtgatatagctgaaacacttgatttactcctacctgctattagtggctactcacttccaaaaggttctcttgactatctcaaaggccttcgtgataatgtagtagaagatgtcaaatttgataagacagtcaaatccggtctggttcatccgcttgatgtgattgatgtccttcaaaagcaaacgactgatgatatgacagtaacggttgatgttggcagccattatatttggatggctcgttattttaaaagctatgaagcacggcacttacttttctcaaatggtatgcaaaccttaggtgttgctttgccttgggcaatttcggcagctcttgtacggccaaatgagaagattatttctatttcaggtgatggtggtttcctcttttctggccaagaattggaaacagctgttcgtttacatttaccaattgttcatatcatttggaatgatggtaaatataatatggttgaattccaagaagaaatgaaatacggccgttcagcaggtgttgattttggtcctgttgattttgtcaagtatgctgatagtttcggtgctaaaggttaccgtgctgatagtaaagaaaagtttgatcaagttcttcaaacagcactcaaggaagctgcaaatggcccagttctcattgatgttccaatggactataaagataatgtaaaattgggtgaaactattttgcctgatgaattctactaa

SEQ ID NO: 12 - protein, 559 aa, Streptococcus mutans
MTEINKEGYGADLIVDSLINHDVNYVFGIPGAKIDRVFDTLEDKGPELIVARHEQNAAFMAQGIGRITGEPGVVITTSGPGVSNLVTGLVTATAEGDPVLAIGGQVKRADLLKRAHQSMNNVAMLDPITKYSAEIQDPATLSENIANAYRLAKAGKPGASFLSIPQDITDSPVTVKAIKPLTDPKLGSASVADINYLAQAIKNAVLPVLLLGNGASTAAVTASIRRLLGAVKLPVVETFQGAGIVSRDLEEDTFFGRVGLFRNQPGDMLLKRSDLVIAIGYDPIEYEARNWNAEISARIIVIDVAPAEIDTYFQPERELIGDIAETLDLLLPAISGYSLPKGSLDYLKGLRDNVVEDVKFDKTVKSGLVHPLDVIDVLQKQTTDDMTVTVDVGSHYIWMARYFKSYEARHLLFSNGMQTLGVALPWAISAALVRPNEKIISISGDGGFLFSGQELETAVRLHLPIVHIIWNDGKYNMVEFQEEMKYGRSAGVDFGPVDFVKYADSFGAKGYRADSKEKFDQVLQTALKEAANGPVLIDVPMDYKDNVKLGETILPDEFY

SEQ ID NO: 13 - DNA, 1683 bp, Streptococcus thermophilus
  CDS (1)..(1683)
gtgttcatgtcagaagaaaagcaattgtatggtgcagatttagtggttgatagtttgatcaaccatgatgttgagtatgtctttgggattccaggcgcaaaaatcgatagggtttttgataccttggaagataagggacctgaattgattgttgcccgtcatgagcaaaatgctgcttttatggctcaaggtgttggacgtattactgggaaaccaggtgtagtattggtaacatctggtccaggtgtctccaatttggctactggtttggtaacagcgacggatgaaggagaccctgttcttgctattggtggtcaggttaagcgtgcagatctcttgaaacgtgcccaccaatcaatgaataacgttgctatgcttgagccaattaccaaatatgctgctgaagtacatgatgctaacaccctttctgaaacggttgctaatgcctatcgtcacgctaagtcagggaaaccaggtgcaagcttcatttcaattcctcaagacgtgacggatgctccggtcagtgttaaggctattaagcctatgacagatccaaaacttggttcagcatctgtttctgatattaactatctagcacaagccattaaaaatgcagtgttgccagtctttcttttggggaatggtgcctcatcagaagccgtaacttactctattcgccaaattttgaagcatgttaaattgccagttgttgaaactttccaaggtgccggtatcgtgtcacgtgaccttgaagaagatactttctttggtcgtgtaggtcttttccgtaaccaacccggagacatgttgcttaaaaaatccgacttagttattgccattggttatgatccaatcgaatatgaagcacgtaactggaatgctgaaatttcagcacgtatcatcgttattgatgtcgagccggccgaggtggacacttacttccaaccggaacgtgaattgattggtaatgtagaagcgagcttagacttgcttttgcccgctattcaaggttataaattgcctgaaggtgcggttgaatatcttaaaggtttgaaaaacaatgttgttgaggatgttaagtttgaccgtcagcctgatgaaggtacggtgcatccgctagatttcatcgaaaatttgcaagaacacacagatgatgatatgactgttacgtttgatgttggtagtcactatatttggatggcacgttatctcaaatcgtatgaaccacgtcatttgcttttctcaaatgggatgcaaacgataggtattgctattacatgggctatctctgcagcattggttcgtcctaagacaaaagtgatttctgtatctggtgatggtggtttcctcttctcagcacaagaattggaaacagcagttcgtttgaaattgccaattgtccatattatctggaacgatggtcattacaatatggtggaattccaggaagaaatgaagtacggtcgttcatctggggttgactttggtcctgtagattttgtaaaatatgctgagagctttggagccaaaggttatcgtgcaacaagtaaagcagcgtttgctagcttgcttcaagaggctttgactcaggctgtagatggaccagtccttattgatgttccaattgactataaagataacattaaactcggcgaaactattttgccagatgaattttactaa

SEQ ID NO: 14 - protein, 560 aa, Streptococcus thermophilus
VFMSEEKQLYGADLVVDSLINHDVEYVFGIPGAKIDRVFDTLEDKGPELIVARHEQNAAFMAQGVGRITGKPGVVLVTSGPGVSNLATGLVTATDEGDPVLAIGGQVKRADLLKRAHQSMNNVAMLEPITKYAAEVHDANTLSETVANAYRHAKSGKPGASFISIPQDVTDAPVSVKAIKPMTDPKLGSASVSDINYLAQAIKNAVLPVFLLGNGASSEAVTYSIRQILKHVKLPVVETFQGAGIVSRDLEEDTFFGRVGLFRNQPGDMLLKKSDLVIAIGYDPIEYEARNWNAEISARIIVIDVEPAEVDTYFQPERELIGNVEASLDLLLPAIQGYKLPEGAVEYLKGLKNNVVEDVKFDRQPDEGTVHPLDFIENLQEHTDDDMTVTFDVGSHYIWMARYLKSYEPRHLLFSNGMQTIGIAITWAISAALVRPKTKVISVSGDGGFLFSAQELETAVRLKLPIVHIIWNDGHYNMVEFQEEMKYGRSSGVDFGPVDFVKYAESFGAKGYRATSKAAFASLLQEALTQAVDGPVLIDVPIDYKDNIKLGETILPDEFY

SEQ ID NO: 15 - DNA, 1665 bp, Vibrio angustum
  CDS (1)..(1665)
atgtcggataaaaccgtctctggtgctgaactggttgttgaaactttaaatgcacataacgttccacacatttttggtattcctggagcaaaggtggatgctgttttcgatgctgtttgtgataacggaccagaaatcattatttgtcatcatgaacaaaatgcagcgtttatggcagcagcaactgggcgtttaacgggtaaagcaggcatttgtttagcaacctctggaccaggcgcatcaaaccttgtcacaggcgttgcaacagcgaatagtgaaggtgatcctgtggttgcacttgcaggtgctgtacctctttctatgtattctcacaatactcatcaatccatggatacccgttcactgtttactcctatcaccaagttttcagcagaagtgatggatagcagctcggtatctgatgttgtacataaagcttttcgtattgcagagcaacctacccaaggtgctagctttgttagtctaccgcaagatattctaactaaccgtattccttaccagccagtacaacagcctaatccaattttgttcggtggtgcacacccacaagctattcgtcaggctgctgatcgcattaatgctgcaaaaaatccggtgttattactgggcatggatgcaagccagccttttgttgctgatgctattcgccaactactcaaacaaacaccaattgccgttgtgaatacgtttgccgcagctggggttatttctcatgatttatacaactgctttttaggtcgtgttggcttatttaaaaatcaacccggtgatattgcattaaacagtgcagatttaatcattaccattggctacagcccaattgaatacgatccgattctttggaataaagatgcaaacacaccaattattcatattggttatcaacaagcagatttagaaattagctataaccctgtttgtgaagttgtgggtgacttagcggtgtctgtcacgtctattgcttctgaattagataagcgagaatcattagaaaataaccaacaaatccaattattacgccacgatttacaacatattatgcagatgggggtaaataaaacctcaacaaacggcgttcacccgcttcgttttgttcatgagttacgtcgctttgttagtgacgacaccactgtatgttgtgatgtaggctctatttatatttggatggcacgttacttccacagctttgaacctcgtcgtttattgttcagcaatggccaacaaacattgggcgtagctttaccttgggcaattgcagcttcccttcttcaccctaatgaaaaagtaatttccatgtctggtgatggtggcttcctattctcatcaatggaattagccacggccgttcgccataaatgtaatatcgttcactttgtttggacagatcacagttatgacatggttaagatccaacagcttaaaaagtatggtcgagagagtgccgtcagctttataggtcctgatattgttaagtacgcagaaagcttcggcgcacatggtttagcgatcaatactgccgatgatattgagcctgttatgcgaaaagctatgagcttaagtggcccagtattggtcaacgtcaatgttgattatagcgataacagtcgcctacttgatcaacttcatccatgccaacaagattaa

SEQ ID NO: 16 - protein, 554 aa, Vibrio angustum
MSDKTVSGAELVVETLNAHNVPHIFGIPGAKVDAVFDAVCDNGPEIIICHHEQNAAFMAAATGRLTGKAGICLATSGPGASNLVTGVATANSEGDPVVALAGAVPLSMYSHNTHQSMDTRSLFTPITKFSAEVMDSSSVSDVVHKAFRIAEQPTQGASFVSLPQDILTNRIPYQPVQQPNPILFGGAHPQAIRQAADRINAAKNPVLLLGMDASQPFVADAIRQLLKQTPIAVVNTFAAAGVISHDLYNCFLGRVGLFKNQPGDIALNSADLIITIGYSPIEYDPILWNKDANTPIIHIGYQQADLEISYNPVCEVVGDLAVSVTSIASELDKRESLENNQQIQLLRHDLQHIMQMGVNKTSTNGVHPLRFVHELRRFVSDDTTVCCDVGSIYIWMARYFHSFEPRRLLFSNGQQTLGVALPWAIAASLLHPNEKVISMSGDGGFLFSSMELATAVRHKCNIVHFVWTDHSYDMVKIQQLKKYGRESAVSFIGPDIVKYAESFGAHGLAINTADDIEPVMRKAMSLSGPVLVNVNVDYSDNSRLLDQLHPCQQD

SEQ ID NO: 17 - DNA, 1689 bp, Bacillus cereus
ttgagtacaggtgtaaaagcaaacgacgtgaagacaaaaacaaaaggagcagatcttgttgttgattgtttaattaaacaaggtgttacacatgttttcggtattccaggagcaaagattgactctgtatttgatgtactgcaagaaagaggaccagagttaattgtttgtcgtcatgaacaaaatgcagcatttatggcagctgctattggtagattaacaggaaaaccgggcgtatgtcttgtaacttcaggaccagggacatcaaatttagcgacaggtcttgttactgcgaatgcggagagtgatcccgttgttgctttagctggtgcagttccgcgtacggatcgattaaaacgtacacatcaatctatggataatgctgcactattcgaaccaatcacaaaatatagcgtagaagtagagcatcctgataatgtgccagaagcattatcaaatgcattccgaagtgcgacttctacaaatccaggagcaactttagtaagtttgccgcaagacgttatgactgcggaaacgactgtagagtctatcggtgcgctttctaagccacagcttggaatcgctcccacacatgatattacatatgtagtagataaaataaaagcagcgaaattaccagttattttactcggtatgagagcgagcacaaatgaagtgacgaaagccgttcgtaaattaattgcggatacagaacttcctgtcgttgaaacatatcaagcggctggtgccatttcacgtgagttagaagatcatttcttcggccgtgttggactattccgtaaccaaccaggtgatattttactagaagaggcagatcttgttatttctatcggttatgatccaattgagtatgatccaaagttctggaataaacttggagacagaacgattattcatcttgatgaccatcaagcagatatagatcatgattaccaaccagagcgtgaattaattggtgatattgccttaacagtaaatagcatcgcagaaaagttaccgaaacttgtgttaagtacgaaatcagaagcagtgttagaacgattacgcgcgaaattatcagaacaagcagaagttccaaatcgtccttcagaaggtgttacacatccgcttcaagtgattcgtacacttcgttctttaattagtgacgacacaaccgttacatgtgacatcggttcccattctatttggatggcgagatgtttccgttcttatgaaccacgtagattattatttagtaacggtatgcagacgttaggtgttgcacttccttgggcaattgctgctactttagtagaaccaggtaaaaaagtagtttccgtatcaggtgacggtggtttcttattctcagcgatggagttagaaacggcggtacgtttaaattctccaatcgtccatcttgtttggagagacggcacatatgatatggttgcattccaacaaatgatgaaatacggcagaacatcagctacagagtttggtgatgttgatcttgttaaatatgcggaaagtttcggggcgttaggtcttcgtgttaacacgcctgatgaattagaaggggtattgaaagaagcactagcagcagacggccctgtcattattgatattccaattgactatcgtgacaacattaaattaagcgaaaaattattaccaaaccaattaaactaa

SEQ ID NO: 18 - protein, 562 aa, Bacillus cereus
MSTGVKANDVKTKTKGADLVVDCLIKQGVTHVFGIPGAKIDSVFDVLQERGPELIVCRHEQNAAFMAAAIGRLTGKPGVCLVTSGPGTSNLATGLVTANAESDPVVALAGAVPRTDRLKRTHQSMDNAALFEPITKYSVEVEHPDNVPEALSNAFRSATSTNPGATLVSLPQDVMTAETTVESIGALSKPQLGIAPTHDITYVVDKIKAAKLPVILLGMRASTNEVTKAVRKLIADTELPVVETYQAAGAISRELEDHFFGRVGLFRNQPGDILLEEADLVISIGYDPIEYDPKFWNKLGDRTIIHLDDHQADIDHDYQPERELIGDIALTVNSIAEKLPKLVLSTKSEAVLERLRAKLSEQAEVPNRPSEGVTHPLQVIRTLRSLISDDTTVTCDIGSHSIWMARCFRSYEPRRLLFSNGMQTLGVALPWAIAATLVEPGKKVVSVSGDGGFLFSAMELETAVRLNSPIVHLVWRDGTYDMVAFQQMMKYGRTSATEFGDVDLVKYAESFGALGLRVNTPDELEGVLKEALAADGPVIIDIPIDYRDNIKLSEKLLPNQLN

SEQ ID NO: 19 - DNA, 780 bp, Klebsiella pneumoniae
atgaatcattctgctgaatgcacctgcgaagagagtctatgcgaaaccctgcgggcgttttccgcgcagcatcccgagagcgtgctctatcagacatcgctcatgagcgccctgctgagcggggtttacgaaggcagcaccaccatcgcggacctgctgaaacacggcgatttcggcctcggcacctttaatgagctggacggggagctgatcgccttcagcagtcaggtctatcagctgcgcgccgacggcagcgcgcgcaaagcccagccggagcagaaaacgccgttcgcggtgatgacctggttccagccgcagtaccggaaaacctttgaccatccggtgagccgccagcagctgcacgaggtgatcgaccagcaaatcccctctgacaacctgttctgcgccctgcgcatcgacggccatttccgccatgcccatacccgcaccgtgccgcgccagacgccgccgtaccgggcgatgaccgacgtcctcgacgatcagccggtgttccgctttaaccagcgcgaaggggtgctggtcggcttccggaccccgcagcatatgcaggggatcaacgtcgccgggtatcacgagcactttattaccgatgaccgcaaaggcggcggtcacctgctggattaccagctcgaccatggggtgctgaccttcggcgaaattcacaagctgatgatcgacctgcccgccgacagcgcgttcctgcaggctaatctgcatcccgataatctcgatgccgccatccgttccgtagaaagttaa

SEQ ID NO: 20 - protein, 259 aa, Klebsiella pneumoniae
MNHSAECTCEESLCETLRAFSAQHPESVLYQTSLMSALLSGVYEGSTTIADLLKHGDFGLGTFNELDGELIAFSSQVYQLRADGSARKAQPEQKTPFAVMTWFQPQYRKTFDHPVSRQQLHEVIDQQIPSDNLFCALRIDGHFRHAHTRTVPRQTPPYRAMTDVLDDQPVFRFNQREGVLVGFRTPQHMQGINVAGYHEHFITDDRKGGGHLLDYQLDHGVLTFGEIHKLMIDLPADSAFLQANLHPDNLDAAIRSVES

SEQ ID NO: 21 - DNA, 768 bp, Bacillus subtilis
atgaaacgagaaagcaacattcaagtgctcagccgtggtcaaaaagatcagcctgtgagccagatttatcaagtatcaacaatgacttctctattagacggagtatatgacggagattttgaactgtcagagattccgaaatatggagacttcggtatcggaacctttaacaagcttgacggagagctgattgggtttgacggcgaattttaccgtcttcgctcagacggaaccgcgacaccggtccaaaatggagaccgttcaccgttctgttcatttacgttctttacaccggacatgacgcacaaaattgatgcgaaaatgacacgcgaagactttgaaaaagagatcaacagcatgctgccaagcagaaacttattttatgcaattcgcattgacggattgtttaaaaaggtgcagacaagaacagtagaacttcaagaaaaaccttacgtgccaatggttgaagcggtcaaaacacagccgattttcaacttcgacaacgtgagaggaacgattgtaggtttcttgacaccagcttatgcaaacggaatcgccgtttctggctatcacctgcacttcattgacgaaggacgcaattcaggcggacacgtttttgactatgtgcttgaggattgcacggttacgatttctcaaaaaatgaacatgaatctcagacttccgaacacagcggatttctttaatgcgaatctggataaccctgattttgcgaaagatatcgaaacaactgaaggaagccctgaataa

SEQ ID NO: 22 - protein, 255 aa, Bacillus subtilis
MKRESNIQVLSRGQKDQPVSQIYQVSTMTSLLDGVYDGDFELSEIPKYGDFGIGTFNKLDGELIGFDGEFYRLRSDGTATPVQNGDRSPFCSFTFFTPDMTHKIDAKMTREDFEKEINSMLPSRNLFYAIRIDGLFKKVQTRTVELQEKPYVPMVEAVKTQPIFNFDNVRGTIVGFLTPAYANGIAVSGYHLHFIDEGRNSGGHVFDYVLEDCTVTISQKMNMNLRLPNTADFFNANLDNPDFAKDIETTEGSPE

SEQ ID NO: 23 - DNA, 780 bp, Klebsiella terrigena
gtgaatcattatcctgaatgcacctgccaggagagcctgtgcgaaaccgtacgcggcttctccgcccaccaccctgatagcgttatctatcagacctctctgatgagcgcgctgctgagcggggtctatgagggtagcaccaccatcgccgacctgctgacccacggcgacttcggtctcggcacctttaacgaactcgatggcgaactgattgcctttagcagcgaggtctaccagctgcgcgctgacggcagcgcgcgtaaagcccgggcggatcaaaaaacgcccttcgcggtgatgacctggttcagaccgcagtaccgtaaaacctttgaccacccggtcagccgccagcagctgcacgacgttatcgaccagcaaatcccctccgataacctgttctgcgccctgcatattgatggtcactttcgccacgcccacacccgcaccgtgccgcggcagacgccgccctatcgggcgatgaccgacgtgctcgatgaccagccggttttccgcttcaaccagcgcaaggggacgctggtcggctttcgcaccccgcagcatatgcagggccttaacgttgccggctaccacgagcactttattaccgacgatcgccagggcggcggccatctgctggactaccagctcgatagcggcgtgctgaccttcggcgagatccacaagctgatgattgacctcccggccgacagcgctttcctgcaggccgacctgcatcctgacaatctcgatgccgctattcgtgcggtagaaaactaa

SEQ ID NO: 24 - protein, 259 aa, Klebsiella terrigena
MNHYPECTCQESLCETVRGFSAHHPDSVIYQTSLMSALLSGVYEGSTTIADLLTHGDFGLGTFNELDGELIAFSSEVYQLRADGSARKARADQKTPFAVMTWFRPQYRKTFDHPVSRQQLHDVIDQQIPSDNLFCALHIDGHFRHAHTRTVPRQTPPYRAMTDVLDDQPVFRFNQRKGTLVGFRTPQHMQGLNVAGYHEHFITDDRQGGGHLLDYQLDSGVLTFGEIHKLMIDLPADSAFLQADLHPDNLDAAIRAVEN

SEQ ID NO: 25 - DNA, 771 bp, Klebsiella pneumoniae
atgaaaaaagtcgcacttgttaccggcgccggccaggggattggtaaagctatcgcccttcgtctggtgaaggatggatttgccgtggccattgccgattataacgacgccaccgccaaagcggtcgcctcggaaatcaaccaggccggcggacacgccgtggcggtgaaagtggatgtctccgaccgcgatcaggtatttgccgccgttgaacaggcgcgcaaaacgctgggcggcttcgacgtcatcgtcaataacgccggtgtggcaccgtctacgccgatcgagtccattaccccggagattgtcgacaaagtctacaacatcaacgtcaaaggggtgatctggggtattcaggcggcggtcgaggcctttaagaaagaggggcacggcgggaaaatcatcaacgcctgttcccaggccggccacgtcggcaacccggagctggcggtgtatagctccagtaaattcgcggtacgcggcttaacccagaccgccgctcgcgacctcgcgccgctgggcatcacggtcaacggctactgcccggggattgtcaaaacgccaatgtgggccgaaattgaccgccaggtgtccgaagccgccggtaaaccgctgggctacggtaccgccgagttcgccaaacgcatcactctcggtcgtctgtccgagccggaagatgtcgccgcctgcgtctcctatcttgccagcccggattctgattacatgaccggtcagtcgttgctgatcgacggcgggatggtatttaactaa

SEQ ID NO: 26 - protein, 256 aa, Klebsiella pneumoniae
MKKVALVTGAGQGIGKAIALRLVKDGFAVAIADYNDATAKAVASEINQAGGHAVAVKVDVSDRDQVFAAVEQARKTLGGFDVIVNNAGVAPSTPIESITPEIVDKVYNINVKGVIWGIQAAVEAFKKEGHGGKIINACSQAGHVGNPELAVYSSSKFAVRGLTQTAARDLAPLGITVNGYCPGIVKTPMWAEIDRQVSEAAGKPLGYGTAEFAKRITLGRLSEPEDVAACVSYLASPDSDYMTGQSLLIDGGMVFN

SEQ ID NO: 27 - DNA, 1053 bp, Bacillus cereus
atgaaagcactactttggcataatcaacgtgatgtacgagtagaagaagtaccagaaccaacagtaaaaccaggaacagtgaaaatcaaagttaaatggtgtggtatttgtgggacagacttgcatgaatatttagcagggcctatttttattccaacagaagaacatccattaacacatgtgaaagcacctgttattttaggtcatgagtttagtggtgaggtaatagagattggtgaaggagttacatctcataaagtgggagaccgcgttgttgtagagccaatttattcttgtggtaaatgtgaagcttgtaaacatggacattacaatgtttgtgaacaacttgttttccacggtcttggcggagaaggcggcggtttctctgaatatacagtagtaccagaagatatggttcatcacattccagatgaaatgacgtatgaacaaggtgcgcttgtagaaccagcagcagtagcagttcatgcagtacgtcaaagtaaattaaaagaaggggaagctgtagcggtatttggttgcggtccaattggacttcttgttatccaagcagctaaagcagcaggagcaactcctgttattgcagttgaactttctaaagaacgtcaagagttagcgaaattagcaggtgcggattatgtattaaatccagcaactcaagatgtgttagctgaaattcgtaacttaacaaatggtttaggtgtaaatgttagctttgaagtaacaggtgttgaagttgtactacgccaagcgattgaaagtacaagcttcgaaggacaaactgtaattgttagtgtatgggaaaaagacgcaacaattactccaaataacttagtattaaaagaaaaagaagttattggtattttaggataccgtcacatcttcccagctgttattaaattgattagctccggtcaaattcaagcagagaaattaattacgaaaaaaattacagtggatcaagttgttgaagaaggatttgaagcacttgtaaaagataaaacacaagtgaaaattcttgtttcacctaaataa

SEQ ID NO: 28 - protein, 350 aa, Bacillus cereus
MKALLWHNQRDVRVEEVPEPTVKPGTVKIKVKWCGICGTDLHEYLAGPIFIPTEEHPLTHVKAPVILGHEFSGEVIEIGEGVTSHKVGDRVVVEPIYSCGKCEACKHGHYNVCEQLVFHGLGGEGGGFSEYTVVPEDMVHHIPDEMTYEQGALVEPAAVAVHAVRQSKLKEGEAVAVFGCGPIGLLVIQAAKAAGATPVIAVELSKERQELAKLAGADYVLNPATQDVLAEIRNLTNGLGVNVSFEVTGVEVVLRQAIESTSFEGQTVIVSVWEKDATITPNNLVLKEKEVIGILGYRHIFPAVIKLISSGQIQAEKLITKKITVDQVVEEGFEALVKDKTQVKILVSPK

SEQ ID NO: 29 - DNA, 1113 bp, Lactococcus lactis
ttgcctgaaacgacaaccatcctatatagaggaggcgtttttatgcgcgcagcacgtttttacgaccgcggggatatccgcattgatgaaattaatgaaccaatagtaaaagctggccaagttggcattgatgtggcttggtgtggaatttgtggaacagatctccatgaatttttagatggcccaattttttgtccgtcagcagaacatcctaatccaattactggagaagtaccaccagtcactcttggacatgaaatgtctggggttgtaaattttataggtgaaggagtaagcggacttaaagtaggtgaccatgtcgttgtcgaaccttatatcgttcccgaagggactgatacaagtgaaactggacattataacctctcagaaggctcaaactttattggtttgggcggaaatggtggaggtttggctgaaaaaatttctgttgatgaacgttgggttcacaaaattcctgataacttaccattggatgaagctgctctaattgagccactatcagtcggctatcacgctgttgaacgagcaaatttaagtgaaaagagtacggtattagttgttggtgctggaccaattggactattaactgctgccgttgcaaaagcgcaaggacatactgttatcatcagtgaacctagtggacttcgtcgtaaaaaagcacaagaagcacaagttgctgattatttcttcaatccaattgaagatgacattcaagctaaagttcatgaaattaatgaaaaaggagtggacgcagcctttgaatgtacctctgtccaaccgggatttgacgcttgtctagatgcgattcgtatgggtggaacagttgtcattgtcgcaatttggggcaagcctgctagtgttgatatggcaaaattagtaatcaaagaagctaaccttttaggaacgattgcttataataacactcatccaaaaacaattgatttagtatcaacaggtaaaataaaattggaccaattcatcacagctaaaatcggtttggatgatttgattgataaaggattcgatacgctgattcatcataatgaaacagctgttaaaattttagtttcaccaactggtaaaggtctataa

SEQ ID NO: 30 - protein, 370 aa, Lactococcus lactis
MPETTTILYRGGVFMRAARFYDRGDIRIDEINEPIVKAGQVGIDVAWCGICGTDLHEFLDGPIFCPSAEHPNPITGEVPPVTLGHEMSGVVNFIGEGVSGLKVGDHVVVEPYIVPEGTDTSETGHYNLSEGSNFIGLGGNGGGLAEKISVDERWVHKIPDNLPLDEAALIEPLSVGYHAVERANLSEKSTVLVVGAGPIGLLTAAVAKAQGHTVIISEPSGLRRKKAQEAQVADYFFNPIEDDIQAKVHEINEKGVDAAFECTSVQPGFDACLDAIRMGGTVVIVAIWGKPASVDMAKLVIKEANLLGTIAYNNTHPKTIDLVSTGKIKLDQFITAKIGLDDLIDKGFDTLIHHNETAVKILVSPTGKGL

SEQ ID NO: 31 - DNA, 2532 bp, Roseburia inulinivorans
atgggcaattacgattcaacaccgatagctaaaagtgataggattaaaagattggttgatcatttgtatgctaaaatgcctgaaattgaggccgctagagcagagctaattactgaatcctttaaggccaccgaaggtcaacctgttgttatgagaaaggctagagcttttgaacatatactaaagaatttgccaattatcataagaccagaagaactgattgttggctcaactacaattgcccctagaggttgccaaacgtatccagaattctcatacgagtggttagaggctgaatttgaaactgtcgaaacgcgttcagctgacccattttatatttcagaagaaacgaagaaacgtttgctggctgccgatgcttattggaaaggtaaaacaacctcagagttggcaacttcatatatggccccagaaactctaagagccatgaagcataacttcttcacccctggaaactacttctacaatggtgtcggtcatgtcacagttcaatatgaaacagtattagcaatcggcttgaatggagtaaaagagaaggttaggaaagagatggagaattgtcattttggtgatgccgattatagtacaaagatgtgtttcttggagagcattttaatatcgtgtgatgccgtaatcacttatgctaatagatatgccaagatggccgaggaaatggctgaaaaagaaacagatgctgcaaggaggcaagaactattaacaatcgccagggtttgcaaaaacgttcctgaattcccagccgaaagcttccaggaggcctgccaatccttttggttcatacaacaagtgcttcaaattgaatccagtggtcattcaatttccccaggtagatttgatcaatatatgtatccttattacgaaaaggatttaaaggaaggtagcttaactagggaatatgctcaggaactgatcgattgtatctgggttaagttaaatgatctgaataagtgcagggatgctgcctctgctgagggctttgcaggatattccttatttcaaaacttaatcgttgggggccaaacggttcaaggaagggacgccaccaatgatttgagttttatgtgtatcacggcatctgaacacgtctttttaccgatgccgtcgttgtctataagagtttggcatggtagttccaaagcactgcttatgagagcagctgaattgactagaaccggtataggcttacctgcttattacaatgatgaagtcatcataccagctttggtgcataggggtgctactatggatgaagcaagaaattacaacataataggatgtgtcgaaccgcaggttcctggtaaaactgatggctggcacgatgcagcattctttaacatgtgcagacctttggaaatggtgtttagtaatggttatgataacggtgaaattgcatctatacaaactggtaacgtagaatcttttcagagttttgatgagtttatggaagcttacagaaaacaaatgctatataacatagaacttatggtaaatgccgacaacgcgatagattatgcccacgcaaagttggccccattgccatttgagtcatgtttggttgatgactgtataaagagaggaatgtccgctcaggaaggcggcgcaatctataatttcactggtccacagggctttggtattgcaaacgttgctgatagcttgtatacgattaagaaattggtgttcgaggagaagagaattacgatgggtgaattaaagaaagcgttggaaatgaattatggtaagggtttggatgccacaaccgctggtgacatcgcaatgcaggtcgcgaagggactaaaagatgccggacaggaagtgggtcccgacgtgatcgctaatacaatccgtcaagttcttgaaatggaattaccagaagatgtaagaaagagatatgaagagatccatgaaatgatacttgagttaccaaagtatggtaatgatatagatgaagttgatgaattagctagagaagcagcttacttttacacaagaccattagaaacttttaagaatccaaggggtggcatgtatcaagccggcctttatcccgtgtccgctaatgtgccactaggcgctcaaacgggggccacacccgatggacgtttggcgcatacacccgtggcggatggcgttggtccgacatcaggcttcgatatatccggaccaacagcttcttgcaattctgtcgccaagttggatcatgctatagcctctaatggtaccttatttaatatgaagatgcacccaaccgcaatggcaggtgaaaagggcttagaatccttcatatcgttgatccgtggttatttcgatcaacaaggtatgcacatgcaatttaacgtagtagacagggctacactgcttgatgcgcaggcccaccctgaaaagtattcaggcttaattgtcagagtggcaggttattctgccctttttaccacattgtccaagtcattacaagatgatataatcaaacgtaccgaacaagcagacaatagatag

SEQ ID NO: 32 - protein, 843 aa, Roseburia inulinivorans
MGNYDSTPIAKSDRIKRLVDHLYAKMPEIEAARAELITESFKATEGQPVVMRKARAFEHILKNLPIIIRPEELIVGSTTIAPRGCQTYPEFSYEWLEAEFETVETRSADPFYISEETKKRLLAADAYWKGKTTSELATSYMAPETLRAMKHNFFTPGNYFYNGVGHVTVQYETVLAIGLNGVKEKVRKEMENCHFGDADYSTKMCFLESILISCDAVITYANRYAKMAEEMAEKETDAARRQELLTIARVCKNVPEFPAESFQEACQSFWFIQQVLQIESSGHSISPGRFDQYMYPYYEKDLKEGSLTREYAQELIDCIWVKLNDLNKCRDAASAEGFAGYSLFQNLIVGGQTVQGRDATNDLSFMCITASEHVFLPMPSLSIRVWHGSSKALLMRAAELTRTGIGLPAYYNDEVIIPALVHRGATMDEARNYNIIGCVEPQVPGKTDGWHDAAFFNMCRPLEMVFSNGYDNGEIASIQTGNVESFQSFDEFMEAYRKQMLYNIELMVNADNAIDYAHAKLAPLPFESCLVDDCIKRGMSAQEGGAIYNFTGPQGFGIANVADSLYTIKKLVFEEKRITMGELKKALEMNYGKGLDATTAGDIAMQVAKGLKDAGQEVGPDVIANTIRQVLEMELPEDVRKRYEEIHEMILELPKYGNDIDEVDELAREAAYFYTRPLETFKNPRGGMYQAGLYPVSANVPLGAQTGATPDGRLAHTPVADGVGPTSGFDISGPTASCNSVAKLDHAIASNGTLFNMKMHPTAMAGEKGLESFISLIRGYFDQQGMHMQFNVVDRATLLDAQAHPEKYSGLIVRVAGYSALFTTLSKSLQDDIIKRTEQADNR

SEQ ID NO: 33 - DNA, 794 bp, Roseburia inulinivorans
atgaaagaatatcttaatacttcaggtagaatatttgatatccagaggtattctattcacgatggccctggtgtgcgtacaattgtgtttctaaaaggttgtgcccttagatgcagatggtgctgtaatcctgaaagccaaagcttcgaagttgaaacaatgacgattaatggaaaacctaaagtcatgggtaaagatgttacagtcgccgaggttatgaagacggtagaaagagacatgccttattaccttcaatcaggtggtggtatcaccttatcgggtggcgaatgtactttgcaaccagaattttcccttggcctattgagagctgcaaaggatttgggcatatccacggcaatagagagcatggcgtacgcaaagtacgaagtaatagaaactcttcttccgtatttggatacgtatttaatggacatcaaacatatgaatcctgagaaacataaagaatacactggtcatgataacttgaggatgttagaaaacgccttaagagtcgcgcattctggtcagaccgaactgatcatcagagtacctgtcatcccaggattcaacgcaactgagcaggaactactagatattgcaaaattcgcagatacactgcctggagttagacaaatacacatcttgccatatcataattttggtcagggtaaatacgaaggattgaacagggactatccgatgggggacactgagaaaccctctaatgaacagatgaaagcttttcaagaaatgattcaaaagaacacttccctacattgccaaatcggtggtta

SEQ ID NO: 34 - protein, 264 aa, roseburia inulinivorans
MKEYLNTSGRIFDIQRYSIHDGPGVRTIVFLKGCALRCRWCCNPESQSFEVETMTINGKPKVMGKDVTVAEVMKTVERDMPYYLQSGGGITLSGGECTLQPEFSLGLLRAAKDLGISTAIESMAYAKYEVIETLLPYLDTYLMDIKHMNPEKHKEYTGHDNLRMLENALRVAHSGQTELIIRVPVIPGFNATEQELLDIAKFADTLPGVRQIHILPYHNFGQGKYEGLNRDYPMGDTEKPSNEQMKAFQEMIQKNTSLHCQIGG

SEQ ID NO: 35 - DNA, 1047 bp, Achromobacter xylosoxidans
atgaaagctctggtttatcacggtgaccacaagatctcgcttgaagacaagcccaagcccacccttcaaaagcccacggatgtagtagtacgggttttgaagaccacgatctgcggcacggatctcggcatctacaaaggcaagaatccagaggtcgccgacgggcgcatcctgggccatgaaggggtaggcgtcatcgaggaagtgggcgagagtgtcacgcagttcaagaaaggcgacaaggtcctgatttcctgcgtcacttcttgcggctcgtgcgactactgcaagaagcagctttactcccattgccgcgacggcgggtggatcctgggttacatgatcgatggcgtgcaggccgaatacgtccgcatcccgcatgccgacaacagcctctacaagatcccccagacaattgacgacgaaatcgccgtcctgctgagcgacatcctgcccaccggccacgaaatcggcgtccagtatgggaatgtccagccgggcgatgcggtggctattgtcggcgcgggccccgtcggcatgtccgtactgttgaccgcccagttctactccccctcgaccatcatcgtgatcgacatggacgagaatcgcctccagctcgccaaggagctcggggcaacgcacaccatcaactccggcacggagaacgttgtcgaagccgtgcataggattgcggcagagggagtcgatgttgcgatcgaggcggtgggcataccggcgacttgggacatctgccaggagatcgtcaagcccggcgcgcacatcgccaacgtcggcgtgcatggcgtcaaggttgacttcgagattcagaagctctggatcaagaacctgacgatcaccacgggactggtgaacacgaacacgacgcccatgctgatgaaggtcgcctcgaccgacaagcttccgttgaagaagatgattacccatcgcttcgagctggccgagatcgagcacgcctatcaggtattcctcaatggcgccaaggagaaggcgatgaagatcatcctctcgaacgcaggcgctgcctga

SEQ ID NO: 36 - protein, 348 aa, Achromobacter xylosoxidans
MKALVYHGDHKISLEDKPKPTLQKPTDVVVRVLKTTICGTDLGIYKGKNPEVADGRILGHEGVGVIEEVGESVTQFKKGDKVLISCVTSCGSCDYCKKQLYSHCRDGGWILGYMIDGVQAEYVRIPHADNSLYKIPQTIDDEIAVLLSDILPTGHEIGVQYGNVQPGDAVAIVGAGPVGMSVLLTAQFYSPSTIIVIDMDENRLQLAKELGATHTINSGTENVVEAVHRIAAEGVDVAIEAVGIPATWDICQEIVKPGAHIANVGVHGVKVDFEIQKLWIKNLTITTGLVNTNTTPMLMKVASTDKLPLKKMITHRFELAEIEHAYQVFLNGAKEKAMKIILSNAGAA

SEQ ID NO: 37 - DNA, 1188 bp, Saccharomyces cerevisiae
atgttgagaactcaagccgccagattgatctgcaactcccgtgtcatcactgctaagagaacctttgctttggccacccgtgctgctgcttacagcagaccagctgcccgtttcgttaagccaatgatcactacccgtggtttgaagcaaatcaacttcggtggtactgttgaaaccgtctacgaaagagctgactggccaagagaaaagttgttggactacttcaagaacgacacttttgctttgatcggttacggttcccaaggttacggtcaaggtttgaacttgagagacaacggtttgaacgttatcattggtgtccgtaaagatggtgcttcttggaaggctgccatcgaagacggttgggttccaggcaagaacttgttcactgttgaagatgctatcaagagaggtagttacgttatgaacttgttgtccgatgccgctcaatcagaaacctggcctgctatcaagccattgttgaccaagggtaagactttgtacttctcccacggtttctccccagtcttcaaggacttgactcacgttgaaccaccaaaggacttagatgttatcttggttgctccaaagggttccggtagaactgtcagatctttgttcaaggaaggtcgtggtattaactcttcttacgccgtctggaacgatgtcaccggtaaggctcacgaaaaggcccaagctttggccgttgccattggttccggttacgtttaccaaaccactttcgaaagagaagtcaactctgacttgtacggtgaaagaggttgtttaatgggtggtatccacggtatgttcttggctcaatacgacgtcttgagagaaaacggtcactccccatctgaagctttcaacgaaaccgtcgaagaagctacccaatctctatacccattgatcggtaagtacggtatggattacatgtacgatgcttgttccaccaccgccagaagaggtgctttggactggtacccaatcttcaagaatgctttgaagcctgttttccaagacttgtacgaatctaccaagaacggtaccgaaaccaagagatctttggaattcaactctcaacctgactacagagaaaagctagaaaaggaattagacaccatcagaaacatggaaatctggaaggttggtaaggaagtcagaaagttgagaccagaaaaccaataa

SEQ ID NO: 38 - protein, 395 aa, Saccharomyces cerevisiae
MLRTQAARLICNSRVITAKRTFALATRAAAYSRPAARFVKPMITTRGLKQINFGGTVETVYERADWPREKLLDYFKNDTFALIGYGSQGYGQGLNLRDNGLNVIIGVRKDGASWKAAIEDGWVPGKNLFTVEDAIKRGSYVMNLLSDAAQSETWPAIKPLLTKGKTLYFSHGFSPVFKDLTHVEPPKDLDVILVAPKGSGRTVRSLFKEGRGINSSYAVWNDVTGKAHEKAQALAVAIGSGYVYQTTFEREVNSDLYGERGCLMGGIHGMFLAQYDVLRENGHSPSEAFNETVEEATQSLYPLIGKYGMDYMYDACSTTARRGALDWYPIFKNALKPVFQDLYESTKNGTETKRSLEFNSQPDYREKLEKELDTIRNMEIWKVGKEVRKLRPENQ

SEQ ID NO: 39 - DNA, 1485 bp, Vibrio cholerae
atggcgaattatttcaatacgctgaatctgcgtgaacagttggatcaacttggtcgttgccgttttatggcgcgagaagagtttgcaaccgaagctgattacctaaaaggtaagaaagtggtgatcgtaggttgtggggctcaaggcctaaaccaaggcctcaatatgcgtgattcaggtttggatgtttcttacgctctgcgtcaggctgcgattgatgaacagcgtcagtcatttaagaatgccaagaataatggcttcaacgtgggtagttatgaacaactcatcccaaccgcagatttggtgattaacttgacgccagacaagcagcacaccagtgtggtcaatgcggtgatgcctctgatgaagcaaggtgctgccttgggttactcacacggttttaatatcgttgaagagggcatgcagatccgtaaagacatcacggttgtgatggtggcaccaaaatgtccgggtacggaagttcgtgaagagtataagcgcggtttcggcgttcctactcttatcgcggtacaccctgaaaacgatccacaaggtgaaggttgggaaattgctaaagcgtgggctgcggcaacgggtggccatcgtgcgggctgtttagcttcttcttttgtggcggaagtgaaatccgatttgatgggtgagcaaaccattctctgcggtatgctgcaagcgggctctatcgtttgttacgagaaaatggttgctgatggcatcgaccctggttatgcgggcaagcttttgcaatttggttgggaaaccattaccgaagcactcaagtttggcggtattactcatatgatggatcgcctgtctaaccctgcaaaaatcaaagcgtttgagctgtctgaagagttgaaagatctgatgcgcccactgtacaacaagcatatggatgacatcatttctggccacttctctagcaccatgatggcggattgggcgaatgatgataaagacttattcggctggcgtgcagaaaccgctgagacgacctttgaaaactatccaacaaccgacgtaaaaattgctgagcaagaatactttgataacggtattttgatgattgccatggtgcgtgctggggttgagttggcgtttgaagcgatgacggcttcaggcatcatcgatgagtcggcttactatgaatcactgcacgaactcccactgattgccaatacggtagcgcgtaagcgtctgtatgaaatgaacgtggtaatctctgacactgctgagtacggtaactatctgtttgccaatgtggcggtaccactattgcgtgaaaagtttatgccgaaagtgggcactgatgtgattggtaaaggattaggcgtggtctctaatcaagttgataacgcaacgcttatcgaagtaaacagcatcatccgtaaccatccggttgagtatatcggtgaagagctacgcggttacatgaaagacatgaagcgcatcgccgtgggtgattaa

SEQ ID NO: 40 - protein, 494 aa, Vibrio cholerae
MANYFNTLNLREQLDQLGRCRFMAREEFATEADYLKGKKVVIVGCGAQGLNQGLNMRDSGLDVSYALRQAAIDEQRQSFKNAKNNGFNVGSYEQLIPTADLVINLTPDKQHTSVVNAVMPLMKQGAALGYSHGFNIVEEGMQIRKDITVVMVAPKCPGTEVREEYKRGFGVPTLIAVHPENDPQGEGWEIAKAWAAATGGHRAGCLASSFVAEVKSDLMGEQTILCGMLQAGSIVCYEKMVADGIDPGYAGKLLQFGWETITEALKFGGITHMMDRLSNPAKIKAFELSEELKDLMRPLYNKHMDDIISGHFSSTMMADWANDDKDLFGWRAETAETTFENYPTTDVKIAEQEYFDNGILMIAMVRAGVELAFEAMTASGIIDESAYYESLHELPLIANTVARKRLYEMNVVISDTAEYGNYLFANVAVPLLREKFMPKVGTDVIGKGLGVVSNQVDNATLIEVNSIIRNHPVEYIGEELRGYMKDMKRIAVGD

SEQ ID NO: 41 - DNA, 1014 bp, Pseudomonas aeruginosa
atgcgcgttttctacgataaagactgtgacctctcgatcatccagggcaagaaagttgccatcatcggctacggctcccagggccacgcccatgcctgcaacctgaaggactccggcgtcgacgtcaccgtgggcctgcgtagcggctccgccaccgtggccaaggccgaagcgcacggtctgaaggttgccgacgtgaagaccgccgtcgccgcagccgacgtggtcatgatcctcaccccggacgagttccagggccgcctgtacaaggaagagatcgagccgaacctgaagaagggcgccaccctggccttcgctcacggcttctccatccactacaaccaggtcgtcccgcgcgccgacctcgacgtgatcatgatcgcgccgaaggcaccgggtcacaccgtgcgttccgagttcgtcaagggcggtggcatccctgacctgatcgccatctaccaggacgcttccggcaacgccaagaacgtcgccctgtcctacgcctgcggcgtcggcggcggtcgtaccggtatcatcgaaaccaccttcaaggacgagaccgaaaccgacctgttcggtgagcaggccgttctctgcggtggttgcgtcgagctggtcaaggccggtttcgaaaccctggtcgaagccggttacgcgccggaaatggcctacttcgagtgcctgcacgagctgaagctgatcgtcgacctgatgtacgaaggcggcatcgccaacatgaactactccatctccaacaatgccgaatacggtgagtacgtaaccggtccggaggtgatcaacgccgagtcccgtgctgccatgcgcaacgccctgaagcgcatccaggacggcgagtacgcgaaaatgttcattaccgaaggtgcggccaactacccgtcgatgactgcctaccgccgcaacaacgccgctcacccgatcgagcagatcggcgagaagctgcgcgcgatgatgccgtggatcgcagccaacaagatcgtcgacaagagcaagaac

SEQ ID NO: 42 - protein, 338 aa, Pseudomonas aeruginosa
MRVFYDKDCDLSIIQGKKVAIIGYGSQGHAHACNLKDSGVDVTVGLRSGSATVAKAEAHGLKVADVKTAVAAADVVMILTPDEFQGRLYKEEIEPNLKKGATLAFAHGFSIHYNQVVPRADLDVIMIAPKAPGHTVRSEFVKGGGIPDLIAIYQDASGNAKNVALSYACGVGGGRTGIIETTFKDETETDLFGEQAVLCGGCVELVKAGFETLVEAGYAPEMAYFECLHELKLIVDLMYEGGIANMNYSISNNAEYGEYVTGPEVINAESRAAMRNALKRIQDGEYAKMFITEGAANYPSMTAYRRNNAAHPIEQIGEKLRAMMPWIAANKIVDKSKN

SEQ ID NO: 43 - DNA, 1014 bp, Pseudomonas fluorescens
atgaaagttttctacgataaagactgcgacctgtcgatcatccaaggtaagaaagttgccatcatcggctacggttcccagggccacgctcaagcatgcaacctgaaggattccggcgtagacgtgactgttggcctgcgtaaaggctcggctaccgttgccaaggctgaagcccacggcttgaaagtgaccgacgttgctgcagccgttgccggtgccgacttggtcatgatcctgaccccggacgagttccagtcccagctgtacaagaacgaaatcgagccgaacatcaagaagggcgccactctggccttctcccacggcttcgcgatccactacaaccaggttgtgcctcgtgccgacctcgacgtgatcatgatcgcgccgaaggctccaggccacaccgtacgttccgagttcgtcaagggcggtggtattcctgacctgatcgcgatctaccaggacgcttccggcaacgccaagaacgttgccctgtcctacgccgcaggcgtgggcggcggccgtaccggcatcatcgaaaccaccttcaaggacgagactgaaaccgacctgttcggtgagcaggctgttctgtgtggcggtaccgtcgagctggtcaaagccggtttcgaaaccctggttgaagctggctacgctccagaaatggcctacttcgagtgcctgcacgaactgaagctgatcgttgacctcatgtacgaaggcggtatcgccaacatgaactactcgatctccaacaacgctgaatacggcgagtacgtgactggtccagaagtcatcaacgccgaatcccgtcaggccatgcgcaatgctctgaagcgcatccaggacggcgaatacgcgaagatgttcatcagcgaaggcgctaccggctacccatcgatgaccgccaagcgtcgtaacaacgctgctcacggtatcgaaatcatcggcgagcaactgcgctcgatgatgccttggatcggtgccaacaaaatcgtcgacaaagccaagaac

SEQ ID NO: 44 - protein, 338 aa, Pseudomonas fluorescens
MKVFYDKDCDLSIIQGKKVAIIGYGSQGHAQACNLKDSGVDVTVGLRKGSATVAKAEAHGLKVTDVAAAVAGADLVMILTPDEFQSQLYKNEIEPNIKKGATLAFSHGFAIHYNQVVPRADLDVIMIAPKAPGHTVRSEFVKGGGIPDLIAIYQDASGNAKNVALSYAAGVGGGRTGIIETTFKDETETDLFGEQAVLCGGTVELVKAGFETLVEAGYAPEMAYFECLHELKLIVDLMYEGGIANMNYSISNNAEYGEYVTGPEVINAESRQAMRNALKRIQDGEYAKMFISEGATGYPSMTAKRRNNAAHGIEIIGEQLRSMMPWIGANKIVDKAKN

SEQ ID NO: 45 - DNA, 1014 bp, artificial sequence
  Mutant of Pseudomonas fluorescens ilvC coding region
atgaaggtgttttacgataaagactgcgatctgagcatcatccagggaaagaaggttgctattataggatatggttcccaaggacacgcacaagccttgaacttgaaagattctggggtcgacgtgacagtaggtctgtataaaggtgctgctgatgcagcaaaggctgaagcacatggctttaaagtcacagatgttgcagcggctgttgctggcgctgatttagtcatgattttaattccagatgaatttcaatcgcaattgtacaaaaatgaaatagaaccaaacattaagaagggcgctaccttggccttcagtcatggatttgccattcattacaatcaagtagtccccagggcagatttggacgttattatgattgcacctaaggctccggggcatactgttaggagcgaatttgttaagggtggtggtattccagatttgatcgctatataccaagacgttagcggaaacgctaagaatgtagctttaagctacgcagcaggagttggtggcgggagaacgggtataatagaaaccacttttaaagacgagactgagacagatttatttggagaacaagcggttctgtgcggaggaactgttgaattggttaaagcaggctttgagacgcttgtcgaagcagggtacgctcccgaaatggcatacttcgaatgtctacatgaattgaagttgatagtagacttaatgtatgaaggtggtatagctaatatgaactattccatttcaaataatgcagaatatggtgagtatgtcaccggacctgaagtcattaacgcagaatcaagacaagccatgagaaatgccttgaaacgtatccaggacggtgaatacgctaagatgttcataagtgaaggcgctacgggttacccgagtatgactgctaaaagaagaaacaatgcagcacatggtatcgaaattattggtgaacagttaaggtctatgatgccctggatcggtgctaataagatcgtagacaaggcgaaaaat

SEQ ID NO: 46 - protein, 338 aa, artificial sequence
  mutant of Pseudomonas fluorescens protein
MKVFYDKDCDLSIIQGKKVAIIGYGSQGHAQALNLKDSGVDVTVGLYKGAADAAKAEAHGFKVTDVAAAVAGADLVMILIPDEFQSQLYKNEIEPNIKKGATLAFSHGFAIHYNQVVPRADLDVIMIAPKAPGHTVRSEFVKGGGIPDLIAIYQDVSGNAKNVALSYAAAVGGGRTGIIETTFKDETETDLFGEQAVLCGGTVELVKAGFETLVEAGYAPEMAYFECLHELKLIVDLMYEGGIANMNYSISNNAEYGEYVTGPEVINAESRQAMRNALKRIQDGEYAKMFISEGATGYPSMTAKRRNNAAHGIEIIGEQLRSMMPWIGANKIVDKAKN

SEQ ID NO: 47 - DNA, 1758 bp, Saccharomyces cerevisiae
atgggcttgttaacgaaagttgctacatctagacaattctctacaacgagatgcgttgcaaagaagctcaacaagtactcgtatatcatcactgaacctaagggccaaggtgcgtcccaggccatgctttatgccaccggtttcaagaaggaagatttcaagaagcctcaagtcggggttggttcctgttggtggtccggtaacccatgtaacatgcatctattggacttgaataacagatgttctcaatccattgaaaaagcgggtttgaaagctatgcagttcaacaccatcggtgtttcagacggtatctctatgggtactaaaggtatgagatactcgttacaaagtagagaaatcattgcagactcctttgaaaccatcatgatggcacaacactacgatgctaacatcgccatcccatcatgtgacaaaaacatgcccggtgtcatgatggccatgggtagacataacagaccttccatcatggtatatggtggtactatcttgcccggtcatccaacatgtggttcttcgaagatctctaaaaacatcgatatcgtctctgcgttccaatcctacggtgaatatatttccaagcaattcactgaagaagaaagagaagatgttgtggaacatgcatgcccaggtcctggttcttgtggtggtatgtatactgccaacacaatggcttctgccgctgaagtgctaggtttgaccattccaaactcctcttccttcccagccgtttccaaggagaagttagctgagtgtgacaacattggtgaatacatcaagaagacaatggaattgggtattttacctcgtgatatcctcacaaaagaggcttttgaaaacgccattacttatgtcgttgcaaccggtgggtccactaatgctgttttgcatttggtggctgttgctcactctgcgggtgtcaagttgtcaccagatgatttccaaagaatcagtgatactacaccattgatcggtgacttcaaaccttctggtaaatacgtcatggccgatttgattaacgttggtggtacccaatctgtgattaagtatctatatgaaaacaacatgttgcacggtaacacaatgactgttaccggtgacactttggcagaacgtgcaaagaaagcaccaagcctacctgaaggacaagagattattaagccactctcccacccaatcaaggccaacggtcacttgcaaattctgtacggttcattggcaccaggtggagctgtgggtaaaattaccggtaaggaaggtacttacttcaagggtagagcacgtgtgttcgaagaggaaggtgcctttattgaagccttggaaagaggtgaaatcaagaagggtgaaaaaaccgttgttgttatcagatatgaaggtccaagaggtgcaccaggtatgcctgaaatgctaaagccttcctctgctctgatgggttacggtttgggtaaagatgttgcattgttgactgatggtagattctctggtggttctcacgggttcttaatcggccacattgttcccgaagccgctgaaggtggtcctatcgggttggtcagagacggcgatgagattatcattgatgctgataataacaagattgacctattagtctctgataaggaaatggctcaacgtaaacaaagttgggttgcacctccacctcgttacacaagaggtactctatccaagtatgctaagttggtttccaacgcttccaacggttgtgttttagatgcttga

SEQ ID NO: 48 - protein, 585 aa, Saccharomyces cerevisiae
MGLLTKVATSRQFSTTRCVAKKLNKYSYIITEPKGQGASQAMLYATGFKKEDFKKPQVGVGSCWWSGNPCNMHLLDLNNRCSQSIEKAGLKAMQFNTIGVSDGISMGTKGMRYSLQSREIIADSFETIMMAQHYDANIAIPSCDKNMPGVMMAMGRHNRPSIMVYGGTILPGHPTCGSSKISKNIDIVSAFQSYGEYISKQFTEEEREDVVEHACPGPGSCGGMYTANTMASAAEVLGLTIPNSSSFPAVSKEKLAECDNIGEYIKKTMELGILPRDILTKEAFENAITYVVATGGSTNAVLHLVAVAHSAGVKLSPDDFQRISDTTPLIGDFKPSGKYVMADLINVGGTQSVIKYLYENNMLHGNTMTVTGDTLAERAKKAPSLPEGQEIIKPLSHPIKANGHLQILYGSLAPGGAVGKITGKEGTYFKGRARVFEEEGAFIEALERGEIKKGEKTVVVIRYEGPRGAPGMPEMLKPSSALMGYGLGKDVALLTDGRFSGGSHGFLIGHIVPEAAEGGPIGLVRDGDEIIIDADNNKIDLLVSDKEMAQRKQSWVAPPPRYTRGTLSKYAKLVSNASNGCVLDA

SEQ ID NO: 49 - DNA, 1713 bp, Streptococcus mutans
atgactgacaaaaaaactcttaaagacttaagaaatcgtagttctgtttacgattcaatggttaaatcacctaatcgtgctatgttgcgtgcaactggtatgcaagatgaagactttgaaaaacctatcgtcggtgtcatttcaacttgggctgaaaacacaccttgtaatatccacttacatgactttggtaaactagccaaagtcggtgttaaggaagctggtgcttggccagttcagttcggaacaatcacggtttctgatggaatcgccatgggaacccaaggaatgcgtttctccttgacatctcgtgatattattgcagattctattgaagcagccatgggaggtcataatgcggatgcttttgtagccattggcggttgtgataaaaacatgcccggttctgttatcgctatggctaacatggatatcccagccatttttgcttacggcggaacaattgcacctggtaatttagacggcaaagatatcgatttagtctctgtctttgaaggtgtcggccattggaaccacggcgatatgaccaaagaagaagttaaagctttggaatgtaatgcttgtcccggtcctggaggctgcggtggtatgtatactgctaacacaatggcgacagctattgaagttttgggacttagccttccgggttcatcttctcacccggctgaatccgcagaaaagaaagcagatattgaagaagctggtcgcgctgttgtcaaaatgctcgaaatgggcttaaaaccttctgacattttaacgcgtgaagcttttgaagatgctattactgtaactatggctctgggaggttcaaccaactcaacccttcacctcttagctattgcccatgctgctaatgtggaattgacacttgatgatttcaatactttccaagaaaaagttcctcatttggctgatttgaaaccttctggtcaatatgtattccaagacctttacaaggtcggaggggtaccagcagttatgaaatatctccttaaaaatggcttccttcatggtgaccgtatcacttgtactggcaaaacagtcgctgaaaatttgaaggcttttgatgatttaacacctggtcaaaaggttattatgccgcttgaaaatcctaaacgtgaagatggtccgctcattattctccatggtaacttggctccagacggtgccgttgccaaagtttctggtgtaaaagtgcgtcgtcatgtcggtcctgctaaggtctttaattctgaagaagaagccattgaagctgtcttgaatgatgatattgttgatggtgatgttgttgtcgtacgttttgtaggaccaaagggcggtcctggtatgcctgaaatgctttccctttcatcaatgattgttggtaaagggcaaggtgaaaaagttgcccttctgacagatggccgcttctcaggtggtacttatggtcttgtcgtgggtcatatcgctcctgaagcacaagatggcggtccaatcgcctacctgcaaacaggagacatagtcactattgaccaagacactaaggaattacactttgatatctccgatgaagagttaaaacatcgtcaagagaccattgaattgccaccgctctattcacgcggtatccttggtaaatatgctcacatcgtttcgtctgcttctaggggagccgtaacagacttttggaagcctgaagaaactggcaaaaaa

SEQ ID NO: 50 - protein, 571 aa, Streptococcus mutans
MTDKKTLKDLRNRSSVYDSMVKSPNRAMLRATGMQDEDFEKPIVGVISTWAENTPCNIHLHDFGKLAKVGVKEAGAWPVQFGTITVSDGIAMGTQGMRFSLTSRDIIADSIEAAMGGHNADAFVAIGGCDKNMPGSVIAMANMDIPAIFAYGGTIAPGNLDGKDIDLVSVFEGVGHWNHGDMTKEEVKALECNACPGPGGCGGMYTANTMATAIEVLGLSLPGSSSHPAESAEKKADIEEAGRAVVKMLEMGLKPSDILTREAFEDAITVTMALGGSTNSTLHLLAIAHAANVELTLDDFNTFQEKVPHLADLKPSGQYVFQDLYKVGGVPAVMKYLLKNGFLHGDRITCTGKTVAENLKAFDDLTPGQKVIMPLENPKREDGPLIILHGNLAPDGAVAKVSGVKVRRHVGPAKVFNSEEEAIEAVLNDDIVDGDVVVVRFVGPKGGPGMPEMLSLSSMIVGKGQGEKVALLTDGRFSGGTYGLVVGHIAPEAQDGGPIAYLQTGDIVTIDQDTKELHFDISDEELKHRQETIELPPLYSRGILGKYAHIVSSASRGAVTDFWKPEETGKK

SEQ ID NO: 51 - DNA, 1647 bp, Lactococcus lactis
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 52 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 53 - DNA, 1644 bp, artificial sequence
  codon optimized L. lactis kivD coding region for S. cerevisiae expression
atgtatacagtaggtgactatctgttggacagattacacgaattaggtatagaagaaatattcggagtaccaggtgactacaatttgcaatttctagatcaaattatttcacacaaagatatgaaatgggtgggaaatgctaatgagttaaatgcctcctatatggccgacgggtacgcaagaacgaaaaaggctgcggcattcttgactacatttggtgttggcgaattatccgcagttaatggcttagcgggctcctatgctgagaacctgcctgttgttgagatcgtgggatctcctacctcgaaagtgcagaacgaaggtaagtttgttcaccatacgttggctgatggtgatttcaagcactttatgaagatgcacgaaccggttactgctgccaggactttattgacagccgagaatgcaactgttgaaattgatagagtgttgtctgccttactaaaggaaagaaagccggtttacatcaatttacctgtagatgtagctgccgctaaggctgaaaaaccatccttgcctcttaagaaggaaaattccacgtcgaatacatctgatcaagagattctgaacaaaatacaggaaagtctgaagaatgccaagaaaccaattgtaatcacaggccatgaaattatatcgttcggcctagagaagactgttactcagtttatttcaaagactaagttacctattactactttgaactttggtaaatcatctgttgatgaagcattgccctcatttttggggatttacaacggtactctgtcagagccaaacttgaaggaatttgtggaatctgctgattttattcttatgttgggtgtaaagcttaccgattctagtacgggtgcatttactcaccatcttaatgaaaataaaatgatttccttgaatatcgatgaaggtaaaattttcaacgaaagaatccaaaatttcgacttcgaatccctgatatcatctcttcttgacttgtccgaaattgaatataaaggcaagtacatagataaaaagcaagaagattttgtaccttctaacgcgctgttgtcacaagatagactgtggcaagctgtcgaaaatttgacccaaagtaatgagacgatcgtggctgaacaaggcacttctttcttcggtgcctcatctatatttctgaaatcgaaatcacattttattggtcaacccttgtggggatctataggatacactttccccgcagctctaggcagccaaattgcagataaagaatctagacatttattgtttatcggagatggatcattgcaactgactgtccaagaattaggactagccattagagagaagataaacccaatctgctttatcattaataacgatggttacacggttgagagggaaattcatggtccgaaccagagttataatgacattcctatgtggaattactcaaaactgccagaaagtttcggggcaacggaagacagagttgtgtccaaaattgtgagaacagaaaatgaattcgtatccgtgatgaaagaagctcaagcagatccaaataggatgtattggatagaacttattctagcaaaggagggtgcacctaaagttttgaaaaagatgggtaagttatttgcagaacaaaacaagagc

SEQ ID NO: 54 - DNA, 1146 bp, Saccharomyces cerevisiae
atgagagctttggcatatttcaagaagggtgatattcacttcactaatgatatccctaggccagaaatccaaaccgacgatgaggttattatcgacgtctcttggtgtgggatttgtggctcggatcttcacgagtacttggatggtccaatcttcatgcctaaagatggagagtgccataaattatccaacgctgctttacctctggcaatgggccatgagatgtcaggaattgtttccaaggttggtcctaaagtgacaaaggtgaaggttggcgaccacgtggtcgttgatgctgccagcagttgtgcggacctgcattgctggccacactccaaattttacaattccaaaccatgtgatgcttgtcagaggggcagtgaaaatctatgtacccacgccggttttgtaggactaggtgtgatcagtggtggctttgctgaacaagtcgtagtctctcaacatcacattatcccggttccaaaggaaattcctctagatgtggctgctttagttgagcctctttctgtcacctggcatgctgttaagatttctggtttcaaaaaaggcagttcagccttggttcttggtgcaggtcccattgggttgtgtaccattttggtacttaagggaatgggggctagtaaaattgtagtgtctgaaattgcagagagaagaatagaaatggccaagaaactgggcgttgaggtgttcaatccctccaagcacggtcataaatctatagagatactacgtggtttgaccaagagccatgatgggtttgattacagttatgattgttctggtattcaagttactttcgaaacctctttgaaggcattaacattcaaggggacagccaccaacattgcagtttggggtccaaaacctgtcccattccaaccaatggatgtgactctccaagagaaagttatgactggttcgatcggctatgttgtcgaagccttcgaagaagttgttcgtgccatccacaacggagacatcgccatggaagattgtaagcaactaatcactggtaagcaaaggattgaggacggttgggaaaagggattccaagagttgatggatcacaaggaatccaacgttaagattctattgacgcctaacaatcacggtgaaatgaag

SEQ ID NO: 55 - protein, 382 aa, Saccharomyces cerevisiae
MRALAYFKKGDIHFTNDIPRPEIQTDDEVIIDVSWCGICGSDLHEYLDGPIFMPKDGECHKLSNAALPLAMGHEMSGIVSKVGPKVTKVKVGDHVVVDAASSCADLHCWPHSKFYNSKPCDACQRGSENLCTHAGFVGLGVISGGFAEQVVVSQHHIIPVPKEIPLDVAALVEPLSVTWHAVKISGFKKGSSALVLGAGPIGLCTILVLKGMGASKIVVSEIAERRIEMAKKLGVEVFNPSKHGHKSIEILRGLTKSHDGFDYSYDCSGIQVTFETSLKALTFKGTATNIAVWGPKPVPFQPMDVTLQEKVMTGSIGYVVEAFEEVVRAIHNGDIAMEDCKQLITGKQRIEDGWEKGFQELMDHKESNVKILLTPNNHGEMK

SEQ ID NO: 56 - DNA, 1125 bp, artificial sequence
  horse ADH coding region codon optimized for S. cerevisiae expression
atgtcaacagccggtaaagttattaagtgtaaagcggcagttttgtgggaagagaaaaagccgtttagcatagaagaagtagaagtagcgccaccaaaagcacacgaggttagaatcaagatggttgccaccggaatctgtagatccgacgaccatgtggtgagtggcactctagttactcctttgccagtaatcgcgggacacgaggctgccggaatcgttgaatccataggtgaaggtgttaccactgttcgtcctggtgataaagtgatcccactgttcactcctcaatgtggtaagtgtagagtctgcaaacatcctgagggtaatttctgccttaaaaatgatttgtctatgcctagaggtactatgcaggatggtacaagcagatttacatgcagagggaaacctatacaccatttccttggtacttctacattttcccaatacacagtggtggacgagatatctgtcgctaaaatcgatgcagcttcaccactggaaaaagtttgcttgatagggtgcggattttccaccggttacggttccgcagttaaagttgcaaaggttacacagggttcgacttgtgcagtattcggtttaggaggagtaggactaagcgttattatggggtgtaaagctgcaggcgcagcgaggattataggtgtagacatcaataaggacaaatttgcaaaagctaaggaggtcggggctactgaatgtgttaaccctcaagattataagaaaccaatacaagaagtccttactgaaatgtcaaacggtggagttgatttctcttttgaagttataggccgtcttgatactatggtaactgcgttgtcctgctgtcaagaggcatatggagtcagtgtgatcgtaggtgttcctcctgattcacaaaatttgtcgatgaatcctatgctgttgctaagcggtcgtacatggaagggagctatatttggcggttttaagagcaaggatagtgttccaaaacttgttgccgactttatggcgaagaagtttgctcttgatcctttaattacacatgtattgccattcgagaaaatcaatgaagggtttgatttgttaagaagtggtgaatctattcgtacaattttaactttt

SEQ ID NO: 57 - protein, 375 aa, Equus caballus
MSTAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 58 - DNA, 1020 bp, Lactococcus lactis
atggcagttacaatgtattatgaagatgatgtagaagtatcagcacttgctggaaagcaaattgcagtaatcggttatggttcacaaggacatgctcacgcacagaatttgcgtgattctggtcacaacgttatcattggtgtgcgccacggaaaatcttttgataaagcaaaagaagatggctttgaaacatttgaagtaggagaagcagtagctaaagctgatgttattatggttttggcaccagatgaacttcaacaatccatttatgaagaggacatcaaaccaaacttgaaagcaggttcagcacttggttttgctcacggatttaatatccattttggctatattaaagtaccagaagacgttgacgtctttatggttgcgcctaaggctccaggtcaccttgtccgtcggacttatactgaaggttttggtacaccagctttgtttgtttcacaccaaaatgcaagtggtcatgcgcgtgaaatcgcaatggattgggccaaaggaattggttgtgctcgagtgggaattattgaaacaacttttaaagaagaaacagaagaagatttgtttggagaacaagctgttctatgtggaggtttgacagcacttgttgaagccggttttgaaacactgacagaagctggatacgctggcgaattggcttactttgaagttttgcacgaaatgaaattgattgttgacctcatgtatgaaggtggttttactaaaatgcgtcaatccatctcaaatactgctgagtttggcgattatgtgactggtccacggattattactgacgaagttaaaaagaatatgaagcttgttttggctgatattcaatctggaaaatttgctcaagatttcgttgatgacttcaaagcggggcgtccaaaattaatagcctatcgcgaagctgcaaaaaatcttgaaattgaaaaaattggggcagagctacgtcaagcaatgccattcacacaatctggtgatgacgatgcctttaaaatctatcag

SEQ ID NO: 59 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDEVKKNMKLVLADIQSGKFAQDFVDDFKAGRPKLIAYREAAKNLEIEKIGAELRQAMPFTQSGDDDAFKIYQ

SEQ ID NO: 60 - DNA, 719 bp, Artificial sequence
  UAS(PGK1)-FBA1
aattaccgtcgctcgtgatttgtttgcaaaaagaacaaaactgaaaaaacccagacacgctcgacttcctgtcttcctattgattgcagcttccaatttcgtcacacaacaaggtcctgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaa

SEQ ID NO: 61 - DNA, 636 bp, Artificial sequence
  Promoter UAS(PGK1)-ENO2
aattaccgtcgctcgtgatttgtttgcaaaaagaacaaaactgaaaaaacccagacacgctcgacttcctgtcttcctattgattgcagcttccaatttcgtcacacaacaaggtcctgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataata

SEQ ID NO: 62 - DNA, 979 bp, Artificial sequence
  Promoter UAS(FBA1)-PDC1
tgacagcaggattatcgtaatacgtaatagttgaaaatctcaaaaatgtgtgggtcattacgtaaataatgataggaatgggattcttctatttttcctttttccattctcgcacgccgaaatgcatgcaagtaacctattcaaagtaatatctcatacatgtttcatgagggtaacaacatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 63 - DNA, 994 bp, Artificial sequence
  UAS(PGK1)-PDC1
aattaccgtcgctcgtgatttgtttgcaaaaagaacaaaactgaaaaaacccagacacgctcgacttcctgtcttcctattgattgcagcttccaatttcgtcacacaacaaggtcctgtcgacccgcacgccgaaatgcatgcaagtaacctattcaaagtaatatctcatacatgtttcatgagggtaacaacatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 64 - DNA, 731 bp, Artificial sequence
  UAS(PGK1)-OLE1
aattaccgtcgctcgtgatttgtttgcaaaaagaacaaaactgaaaaaacccagacacgctcgacttcctgtcttcctattgattgcagcttccaatttcgtcacacaacaaggtcctgtcgaccggggcgcccgtgcaaaaactaactccgagcccgggcatgtcccgggttagcgggcccaacaaaggcgcttatctggtgggcttccgtagaagaaaaaaagctgttgagcgagctatttcgggtatcccagccttctctgcagaccgccccagttggcttggctctggtgctgttcgttagcatcacatcgcctgtgacaggcagaggtaataacggcttaaggttctcttcgcatagtcggcagctttctttcggacgttgaacactcaacaaaccttatctagtgcccaaccaggtgtgcttctacgagtcttgctcactcagacacacctatccctattgttacggctatggggatggcacacaaaggtggaaataatagtagttaacaatatatgcagcaaatcatcggctcctggctcatcgagtcttgcaaatcagcatatacatatatatatgggggcagatcttgattcatttattgttctatttccatctttcctacttctgtttccgtttatattttgtattacgtagaatagaacatcatagtaatagatagttgtggtgatcatattataaacagcactaaaacattacaacaaa

SEQ ID NO: 65 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 66 - protein, 343 aa, Artificial sequence
  Anaerostipes caccae KARI variant K9D3
MEECKMAKIYYQEDCNLSLLDGKTIAVIGYGSQGHAHALNAKESGCNVIIGLYEGAKDWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 67 - protein, 343 aa, Artificial sequence
  Anaerostipes caccae KARI variant K9G9
MEECKMAKIYYQEDCNLSLLDGKTIAVIGYGSQGHAHALNAKESGCNVIIGLYEGAKEWKRAEEQGFEVYTAAEAAKKADIIMILINDEKQATMYKNDIEPNLEAGNMLMFAHGFNIHFGCIVPPKDVDVTMIAPKGPGHTVRSEYEEGKGVPCLVAVEQDATGKALDMALAYALAIGGARAGVLETTFRTETETDLFGEQAVLCGGVCALMQAGFETLVEAGYDPRNAYFECIHEMKLIVDLIYQSGFSGMRYSISNTAEYGDYITGPKIITEDTKKAMKKILSDIQDGTFAKDFLVDMSDAGSQVHFKAMRKLASEHPAEVVGEEIRSLYSWSDEDKLINN

SEQ ID NO: 68 - protein, 267 aa, Saccharomyces cerevisiae
MSQGRKAAERLAKKTVLITGASAGIGKATALEYLEASNGDMKLILAARRLEKLEELKKTIDQEFPNAKVHVAQLDITQAEKIKPFIENLPQEFKDIDILVNNAGKALGSDRVGQIATEDIQDVFDTNVTALINITQAVLPIFQAKNSGDIVNLGSIAGRDAYPTGSIYCASKFAVGAFTDSLRKELINTKIRVILIAPGLVETEFSLVRYRGNEEQAKNVYKDTTPLMADDVADLIVYATSRKQNTVIADTLIFPTNQASPHHIFRG

SEQ ID NO: 69 - DNA, 2073 bp, Saccharomyces cerevisiae
atggaaggcttcaatccggctgacatagaacatgcgtcaccgattaattcatctgacagccattcatcctcctttgtatatgctctacccaaaagtgctagtgaatatgtagtcaaccataatgagggtcgtgcaagtgcaagtggaaatccagccgcagtgccgtctcccataatgacactgaatctcaaaagcacacattccctcaatattgatcagcatgttcatacctcaacatcgccgacggaaactattgggcatattcatcatgtggaaaagctgaatcaaaacaatttgattcatctggatccagtacccaactttgaagataagtccgatattaagccttggttgcaaaagattttttatcctcaaggaatagaacttgtgatagaaaggtcggacgcatttaaagttgtcttcaagtgtaaagctgctaaaaggggaaggaacgcgagaaggaaaagaaaagataagcccaaaggacaggaccacgaagacgagaaatccaagatcaatgatgacgaattagaatatgcgagtccttctaatgccacagtaaccaatgggcctcaaacatcgcccgatcaaacatcctccataaagccaaagaaaaaaagatgtgtatcgaggtttaataactgtccgtttagagtacgagctacttattcgttaaagaggaaaagatggagcattgttgtaatggacaataaccattcacatcagctaaagtttaaccctgattccgaagagtacaaaaaattcaaagaaaaattaagaaaggataatgacgtagatgcaatcaagaaattcgacgaattggaatacagaactttggccaatttgcccattccaacagctacaatcccctgtgattgtggtttaacaaatgaaatacaaagtttcaatgtcgtattgcccactaacagtaatgttacttcatcagcatcctcttcaactgtatcgtccatatcccttgattcatcgaatgcatctaaaaggccatgcttaccctctgtaaataacaccggtagtatcaataccaataacgtaaggaaaccgaaaagccagtgtaagaataaagacacactcttaaaaagaaccaccatgcagaactttctcacaactaaatcaaggctgcgtaagaccggtacgccaacatcttcgcaacactcatctacagcattttcaggatatattgatgatcctttcaatttgaatgaaatcttgccactgccggcatccgatttcaagctaaacactgtaacaaatttgaacgaaattgactttacgaacatttttaccaaatcgccgcatccacatagcgggtctacccatccaagacaagtcttcgaccaattggacgattgttcctctatactcttctctccattaactacaaacacgaataatgaatttgaaggagagtcagatgattttgttcattctccatatttgaactcagaggcagatttcagccaaattcttagtagtgctcccccagtccatcatgacccaaatgaaacacatcaggaaaaccaggatattattgatagatttgctaatagttcccaagaacataatgagtatattctacaatatttgacgcactccgatgctgctaaccacaataacatcggcgttccaaacaacaattcacattcgctaaatactcagcataacgtttctgatctgggcaactcacttttaagacaagaagctttagttggcagctcttcaacaaaaatcttcgacgaattgaaatttgtacaaaatggcccacacggttctcaacatcctatagattttcaacatgttgaccatcgtcatctcagctctaatgaacctcaagtacgatcacatcaatatggtccgcaacagcagccaccgcagcaattgcaatatcaccaaaatcagccccacgacggccataaccacgaacagcaccaaacagtacaaaaggatatgcaaacgcatgaatcgctagaaataatgggaaacacattattggaagagttcaaagacattaaaatggtgaacggcgagttgaagtatgtgaagccagaagattag

SEQ ID NO: 70 - protein, 690 aa, Saccharomyces cerevisiae
MEGFNPADIEHASPINSSDSHSSSFVYALPKSASEYVVNHNEGRASASGNPAAVPSPIMTLNLKSTHSLNIDQHVHTSTSPTETIGHIHHVEKLNQNNLIHLDPVPNFEDKSDIKPWLQKIFYPQGIELVIERSDAFKVVFKCKAAKRGRNARRKRKDKPKGQDHEDEKSKINDDELEYASPSNATVTNGPQTSPDQTSSIKPKKKRCVSRFNNCPFRVRATYSLKRKRWSIVVMDNNHSHQLKFNPDSEEYKKFKEKLRKDNDVDAIKKFDELEYRTLANLPIPTATIPCDCGLTNEIQSFNVVLPTNSNVTSSASSSTVSSISLDSSNASKRPCLPSVNNTGSINTNNVRKPKSQCKNKDTLLKRTTMQNFLTTKSRLRKTGTPTSSQHSSTAFSGYIDDPFNLNEILPLPASDFKLNTVTNLNEIDFTNIFTKSPHPHSGSTHPRQVFDQLDDCSSILFSPLTTNTNNEFEGESDDFVHSPYLNSEADFSQILSSAPPVHHDPNETHQENQDIIDRFANSSQEHNEYILQYLTHSDAANHNNIGVPNNNSHSLNTQHNVSDLGNSLLRQEALVGSSSTKIFDELKFVQNGPHGSQHPIDFQHVDHRHLSSNEPQVRSHQYGPQQQPPQQLQYHQNQPHDGHNHEQHQTVQKDMQTHESLEIMGNTLLEEFKDIKMVNGELKYVKPED

SEQ ID NO: 71 - DNA, 1251 bp, Saccharomyces cerevisiae
atgaaagcaaagtcgatgaagagcataatatctgtgccaatatcagtgtctaaaacagggaaaatgaagttaacagcatctcccgataatctagcatctatgatgtctaaagaccaaaataaacttattcatttagacccagttccgtcttttgaggacaggcacgagattaaaccgtggctacagaaaatattctatcctcaaggaatagatatagtcatcgaaagatcagatagtagcaaagttacttttaagtgtaggtcagtacgatcgaaagtagggttgaaccctaaatccaaaggttcctcttcaagatcccatgcatgtccatttagaattagagcagcctactctgtgcggttacagaagtggaacgtggttgttatgaataacatacattcacatgagctgagatttgatttgattactaaaacggatgactataaaaagtttaaggaaaatcttcgtcagaagaacgacgaaaaagccatcaaaacatttgatgaattagaatataaagcaagtctaaacttaccactcgtcacaccaattatttcttgtgactgtgggttaaccaaggaaattgaagctttcaataacatttttctcccactatcaaacccccctttgacgtctaaaaaaaatttgctcaaaacaaataaaaactccgtttctaaaataaaatccagacagatggataattcgaaacctaggccaaggttgaaaacgaagttggatgcggatctacatgataccggatttttagacaatttcaaaacgcggaattcctgtgtaaagatagagaaagaagattctttaacgaacttaaacgaaattgattttacgaacatgttttgcaacgacaatttcatccaaaattacaaccaaggcctgatggaacttttgacagaacctacaccaggtccttcgtcctcttcttgtatacttccttcaacaccaacgaggcccttgtcacaaagcaaaatggatatagccctgtcagaatcaacaacatcttcaccaaattttatggaaacagacgctccttatggagatgagatcattaaagtttccaaagataccaaaagcaacgcaccaactgctgatacagatattgctacgaacttaggcaaagagagaaacgaaaattttggcatgttaaactacaactacgaagcgttgcttcatttcaacgatgagcattttaatgaattaaattctattgacccagccttaatatcaaaatattaa

SEQ ID NO: 72 - protein, 416 aa, Saccharomyces cerevisiae
MKAKSMKSIISVPISVSKTGKMKLTASPDNLASMMSKDQNKLIHLDPVPSFEDRHEIKPWLQKIFYPQGIDIVIERSDSSKVTFKCRSVRSKVGLNPKSKGSSSRSHACPFRIRAAYSVRLQKWNVVVMNNIHSHELRFDLITKTDDYKKFKENLRQKNDEKAIKTFDELEYKASLNLPLVTPIISCDCGLTKEIEAFNNIFLPLSNPPLTSKKNLLKTNKNSVSKIKSRQMDNSKPRPRLKTKLDADLHDTGFLDNFKTRNSCVKIEKEDSLTNLNEIDFTNMFCNDNFIQNYNQGLMELLTEPTPGPSSSSCILPSTPTRPLSQSKMDIALSESTTSSPNFMETDAPYGDEIIKVSKDTKSNAPTADTDIATNLGKERNENFGMLNYNYEALLHFNDEHFNELNSIDPALISKY

SEQ ID NO: 73 - DNA, 363 bp, Saccharomyces cerevisiae
atgacaggtgaaagaattgaaaaggtgaaaataaatgacgaatttgcaaaatcacatttcctaacaactcagtggagagaaacaaagcgccaacgacactataagatgcccgttactgaacagggactccgcgagaggatagaatcagccataccgcaagtttaccatatcattgtgacggatctttcgtacggttgtggtcagtcgtttgatattgtggtggtcagcgacttctttcaaggtaaaagcaaactaatgaggagtcgtgcagtgaacaaggctgtgaaagaagagctgcaggagattcatgcctttagctgcaagtgctacactgaggaggaatggtctaagattgtggtatga

SEQ ID NO: 74 - protein, 120 aa, Saccharomyces cerevisiae
MTGERIEKVKINDEFAKSHFLTTQWRETKRQRHYKMPVTEQGLRERIESAIPQVYHIIVTDLSYGCGQSFDIVVVSDFFQGKSKLMRSRAVNKAVKEELQEIHAFSCKCYTEEEWSKIVV

SEQ ID NO: 75 - DNA, 858 bp, Saccharomyces cerevisiae
atgtgttcttttcaggttccatctgcattttcttttaactacacctcgtactgttataaacgccaccaagcaagatattacacagcagcaaaactttttcaggaaatgcctgttattgaaattaacgatcaagagcaatttacttacctaactaccactgcggccggcgacaagttaatcgtgctttatttccataccagttgggcagaaccatgcaaagcattaaagcaggtttttgaggccattagtaatgagccttccaattccaacgtctctttcttatccattgatgcggacgaaaactcggaaatttcagaactttttgaaatctcagctgttccatattttatcataattcacaaagggacaatcttaaaagaattatccggcgcggatccaaaggagtatgtgtctttattagaagactgcaagaactcagtcaattccggatcatcacaaactcatactatggaaaatgcaaacgtaaatgaggggagtcataatgatgaagacgatgacgacgaagaagaggaagaagaaactgaggagcaaataaacgctagattgactaaattggtcaatgccgcgccggtaatgttatttatgaaggggagcccctctgaacctaaatgcgggttttcgagacaacttgtgggtatcttgagagaacatcaagtaagatttggcttctttgatatattaagagacgaatctgttagacaaaacttgaaaaagttttctgaatggccaactttccctcaactttatataaatggggagtttcaaggcggtttagacattatcaaggaatccttggaggaagaccctgattttttgcagcatgctctccaatcttaa

SEQ ID NO: 76 - protein, 285 aa, Saccharomyces cerevisiae
MCSFQVPSAFSFNYTSYCYKRHQARYYTAAKLFQEMPVIEINDQEQFTYLTTTAAGDKLIVLYFHTSWAEPCKALKQVFEAISNEPSNSNVSFLSIDADENSEISELFEISAVPYFIIIHKGTILKELSGADPKEYVSLLEDCKNSVNSGSSQTHTMENANVNEGSHNDEDDDDEEEEEETEEQINARLTKLVNAAPVMLFMKGSPSEPKCGFSRQLVGILREHQVRFGFFDILRDESVRQNLKKFSEWPTFPQLYINGEFQGGLDIIKESLEEDPDFLQHALQS

SEQ ID NO: 77 - DNA, 969 bp, Saccharomyces cerevisiae
atgtccattgtagcactaaagaacgcagtggtgacccttatacagaaagcgaaaggtagtggtggaacctcagagttgggggggtctgaatcaactcccttgttgaggggtagtaatagcaatagttcaaggcatgataacttatcctcatctagctcggatattatctatggtagaaattcagcgcaggatctagaaaactcaccgatgtcagtagggaaagataataggaatggcgataacggttcggataacgaaaaggcgaacctagggttcttccaatcagtagatcctcgcgtaattagcgatttgattatcgggctaagcgacggtttaaccgtcccctttgctctaacagctggtctatcttcacttggtgatgcgaaattggtcattaccggtggctttgctgagttgatctcaggtgcgatttctatgggccttggtggctatttgggagcgaagagtgaatctgattattatcatgctgaagtaaagaaggagaaaaggaaattttacgataactccaacctaattaacagagaaattgaagacattctgttagagattaaccctaatttctcggacgaaacaatcgtttcgttcatcaaagatttacaaagaacacctgagctaatggttgacttcattatcaggtacggtaggggtctagacgaacctgccgagaatagagaactaatcagtgcagtcactatcggtggcggttatctacttggtgggctagtaccactagtgccatatttttttgtctcagatgtgggcacaggtctcatttattctataatagtcatggttgtaacattattctggtttggttacgtaaagacaaaactatccatgggtagtggcagttcgacttcaaagaaagttacggaaggtgttgaaatggttgttgtgggtggtgtagcagcaggtgcggcttggttctttgttaagttactgggttaa

SEQ ID NO: 78 - protein, 322 aa, Saccharomyces cerevisiae
MSIVALKNAVVTLIQKAKGSGGTSELGGSESTPLLRGSNSNSSRHDNLSSSSSDIIYGRNSAQDLENSPMSVGKDNRNGDNGSDNEKANLGFFQSVDPRVISDLIIGLSDGLTVPFALTAGLSSLGDAKLVITGGFAELISGAISMGLGGYLGAKSESDYYHAEVKKEKRKFYDNSNLINREIEDILLEINPNFSDETIVSFIKDLQRTPELMVDFIIRYGRGLDEPAENRELISAVTIGGGYLLGGLVPLVPYFFVSDVGTGLIYSIIVMVVTLFWFGYVKTKLSMGSGSSTSKKVTEGVEMVVVGGVAAGAAWFFVKLLG

SEQ ID NO: 79 - protein, 347 aa, Beijerinkia indica
MKALVYRGPGQKLVEERQKPELKEPGDAIVKVTKTTICGTDLHILKGDVATCKPGRVLGHEGVGVIESVGSGVTAFQPGDRVLISCISSCGKCSFCRRGMFSHCTTGGWILGNEIDGTQAEYVRVPHADTSLYRIPAGADEEALVMLSDILPTGFECGVLNGKVAPGSSVAIVGAGPVGLAALLTAQFYSPAEIIMIDLDDNRLGLAKQFGATRTVNSTGGNAAAEVKALTEGLGVDTAIEAVGIPATFELCQNIVAPGGTIANVGVHGSKVDLHLESLWSHNVTITTRLVDTATTPMLLKTVQSHKLDPSRLITHRFSLDQILDAYETFGQAASTQALKVIISMEA

(19) United States
(12) Patent Application Publication
(10) Pub. No.: US 2012/0149080 A1
(43) Pub. Date: Jun. 14, 2012

(54) YEAST PRODUCTION CULTURE FOR THE PRODUCTION OF BUTANOL

(75) Inventors: VASANTHA NAGARAJAN, Wilmington, DE (US); Michael G. Bramucci, Boothwyn, PA (US)

(73) Assignee: Butamax(TM) Advanced Biofuels LLC, Wilmington, DE (US)

(21) Appl. No.: 13/162,165

(22) Filed: Jun. 16, 2011

Related U.S. Application Data
(60) Provisional application No. 61/355,733, filed on Jun. 17, 2010.

(51) Int. Cl. C12P 7/16; C12N 1/19; C12N 1/16

(52) U.S. Cl. 435/160; 435/255.7; 435/254.2; 435/255.21; 435/255.4; 435/255.5

(57) ABSTRACT

High cell density cultures of yeast were found to have higher tolerance for butanol in the medium. The high cell density yeast cultures had greater survival and higher glucose utilization than cultures with low cell densities. Production of butanol using yeast in high cell density cultures is thus beneficial for improving butanol production.

### CROSS-REFERENCE TO RELATED APPLICATIONS

This application claims the benefit of priority of U.S. Provisional Application No. 61/355,733, filed Jun. 17, 2010, which is incorporated herein by reference in its entirety.

### REFERENCE TO SEQUENCE LISTING SUBMITTED ELECTRONICALLY

The content of the electronically submitted sequence listing Name: 20110616_CL4590USNA_SEQLIST.txt; Size: 244,365 bytes; and Date of Creation: Jun. 16, 2011 is incorporated herein by reference in its entirety.

### FIELD OF THE INVENTION

The invention relates to the field of industrial microbiology and the production of butanol. Specifically, production cultures of yeast organisms tolerant to high concentrations of butanol have been developed.

### BACKGROUND OF THE INVENTION

Butanol is an important industrial chemical, useful as a fuel additive, as a feedstock chemical in the plastics industry, and as a foodgrade extractant in the food and flavor industry. Each year 10 to 12 billion pounds of butanol are produced by petrochemical means and the need for this commodity chemical will likely increase.

Butanol may be made through chemical synthesis or by fermentation. Isobutanol is produced biologically as a by-product of yeast fermentation. It is a component of “fusel oil” that forms as a result of incomplete metabolism of amino acids by this group of fungi. Isobutanol is specifically produced from catabolism of L-valine and the yield is typically very low. Additionally, recombinant microbial production hosts, expressing a 1-butanol biosynthetic pathway (Donaldson et al., U.S. Patent Application Publication No. US20080182308A1), a 2-butanol biosynthetic pathway (Donaldson et al., U.S. Patent Publication Nos. US 20070259410A1 and US 20070292927), and an isobutanol biosynthetic pathway (Maggio-Hall et al., U.S. Patent Publication No. US 20070092957) have been described.

Biological production of butanols is generally limited by butanol toxicity to the host microorganism used in fermentation for butanol production. Yeasts are typically sensitive to butanol in the medium. Genetic engineering approaches have been used to alter gene expression to increase yeast cell tolerance to butanol. Another approach to improving production is to improve the fermentation process. U.S. Pat. No. 4,765,992 discloses addition of microorganism cell walls before or during fermentation to adsorb substances toxic to yeast which cause cessation of fermentation during alcoholic fermentation. U.S. Pat. No. 4,414,329 discloses using high mineral salts media with at least about 60 to 160 grams per liter of cells in a method to produce single cell protein. U.S. Pat. No. 4,284,724 discloses achieving a 6% to about 20% (dry cell weight) density fermentation by removing fermentation broth, filtering, and recycling the yeast cells to the fermentor.

There remains a need to develop butanol production cultures of yeast in which effects due to butanol sensitivity are reduced, thereby allowing increased butanol yield.

### SUMMARY OF THE INVENTION

The invention provides cultures for production of butanol that have increased tolerance to butanol due to the presence of a high density of yeast cells.

Provided herein is a production culture for the fermentative production of butanol comprising: a) a medium comprising a suitable carbon substrate for the metabolism of yeast; b) a culture of butanol producing yeast cells having a glucose utilization rate of at least about 0.5 gram per gram of dry cell weight per hour (g/gdcw/h); and c) butanol at a concentration of at least about 2% (weight/volume) the medium.

In another embodiment the invention provides a production culture for the fermentative production of butanol comprising: a) a medium comprising a suitable carbon substrate for the metabolism of yeast; b) a culture of butanol producing yeast cells having a cell density of at least about 2.4 gram dry cell weight per liter (gdcw/L); and c) butanol at a concentration of at least about 2% (weight/volume) in the medium.

In yet another embodiment the invention provides a method for the production of butanol comprising preparing a production culture of the invention wherein the yeast comprises a butanol biosynthetic pathway selected from the group consisting of isobutanol pathway and 1-butanol pathway, and fermenting the yeast under conditions wherein butanol is produced.

Provided herein is a production culture for the fermentative production of butanol comprising: a) a medium comprising a suitable carbon substrate for the metabolism of yeast; b) a culture of butanol producing yeast cells having a glucose utilization rate of at least about 0.5 gram per gram of dry cell weight per hour; and c) butanol at a concentration of at least about 2% (w/v) in the medium.

In embodiments, the cell density is at least about 2.4 gram dry cell weight per liter. In embodiments, the cell density is at least about 7 gram dry cell weight per liter. In embodiments, the butanol producing yeast cells have a glucose utilization rate of at least about 1 gram per gram of dry cell weight per hour.

In embodiments, the butanol producing yeast cells were produced by a method comprising: a) mutagenesis; and b) exposure to 3% isobutanol; and c) repeated freeze-thaw cycles. In embodiments, the freeze-thaw cycle is repeated more than twice. In embodiments, the cells are produced by a method comprising at least 5 freeze-thaw cycles.

In embodiments, the butanol producing yeast cells were produced by a method comprising: a) growth of the butanol producing yeast cells in a medium containing ethanol; b) concentrating the cells to a density in the range of 30 gdcw/L; c) exposure to 3% isobutanol; and d) repeated freeze-thaw cycles. In embodiments, the freeze-thaw cycle is repeated more than twice. In embodiments, the cells are produced by a method comprising at least 5 freeze-thaw cycles.

In embodiments, the butanol producing yeast cells were produced by a method comprising: a) growth of the butanol producing yeast cells in a medium containing ethanol; b) serially transferred to a medium containing 0.1% to 2.0% butanol for growth for a minimum of 10 h and maximum of 72 h; c) gradually increasing the butanol concentration in the medium in subsequent passages for increased growth rates of butanol producing cells; and d) pooling the cells with fast growth rate e) exposure to 3% isobutanol; and/or repeated freeze-thaw cycles.

In embodiments, the yeast is crabtree positive and, in embodiments, the yeast is crabtree negative. In embodiments, the yeast is a member of a genus selected from the group consisting of Saccharomyces, Schizosaccharomyces, Hansenula, Candida, Kluyveromyces, Yarrowia, Issatchenkia and Pichia. In embodiments, the yeast comprises a butanol biosynthetic pathway selected from the group consisting of isobutanol pathway and 1-butanol pathway.

In embodiments, the culture is maintained at the following conditions for between 1 hour to about 200 hours: a) temperature that is between about 20° C. and 37° C.; b) dissolved oxygen that is maintained between microaerobic conditions to above 3%; c) carbon substrates in excess provided by liquefied biomass; d) pH that is between about 3 and 7.5; and d) butanol removal selected from vacuum application and liquid-liquid extraction.

In embodiments, the culture has a glucose utilization rate of at least about 0.5 gram per gram of dry cell weight per hour. In embodiments, the cell density is at least about 7 grams dry cell weight per liter. In embodiments, the butanol concentration is at least about 2.5% and the culture has a glucose utilization rate of at least about 0.4 gram per gram of dry cell weight per hour. In embodiments, the yeast comprises a butanol biosynthetic pathway selected from the group consisting of isobutanol pathway, 1-butanol pathway, and 2-butanol pathway.

In embodiments, the culture comprises PNY0602 or PNY0614. In embodiments, the culture comprises PNY0602 or PNY0614 further comprising a butanol biosynthetic pathway. In embodiments, the butanol biosynthetic pathway is an isobutanol biosynthetic pathway.

### BRIEF DESCRIPTION OF THE FIGURES AND SEQUENCE DESCRIPTIONS

The various embodiments of the invention can be more fully understood from the following detailed description, the figures, and the accompanying sequence descriptions, which form a part of this application.

FIG. 1 shows four different 2-butanol biosynthetic pathways.

FIG. 2 shows three different isobutanol biosynthetic pathways.

FIG. 3 a pathway for 1-butanol biosynthesis.

FIG. 4 shows a graph of glucose utilization in high cell density yeast cultures in the presence of isobutanol.

The following biological deposits have been made under the terms of the Budapest Treaty on the International Recognition of the Deposit of Microorganisms for the Purposes of Patent Procedure:

| | International | |
|---|---|---|
| Depositor Identification | Depository | |
| Reference | Designation | Date of Deposit |

Saccharomyces cerevisiae PNY0602
Saccharomyces cerevisiae PNY0614

The following sequences and the sequence listing provided herewith and incorporated by reference herein conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

TABLE 1
SEQ ID Numbers of Expression Coding Regions and Proteins

| | SEQ | SEQ |
|---|---|---|
| | ID NO: | ID NO: |
| | Nucleic | Amino |
| Description | acid | Acid |
| Klebsiella pneumoniae budB (acetolactate | 1 | 2 |
| synthase) | | |
| Bacillus subtilis alsS | 3 | 4 |
| (acetolactate synthase) | | |
| Lactococcus lactis als | 5 | 6 |
| (acetolactate synthase) | | |
| Als Staphylococcus aureus | 7 | 8 |
| Als Listeria monocytogenes | 9 | 10 |
| Als Streptococcus mutans | 11 | 12 |
| Als Streptococcus thermophilus | 13 | 14 |
| Als Vibrio angustum | 15 | 16 |
| Als Bacillus cereus | 17 | 18 |
| budA, acetolactate decarboxylase from Klebsiella | 19 | 20 |
| pneumoniae ATCC 25955 | | |
| alsD, acetolactate decarboxylase from Bacillus | 21 | 22 |
| subtilis | | |
| budA, acetolactate decarboxylase from Klebsiella | 23 | 24 |
| terrigena | | |
| budC, butanediol dehydrogenase from Klebsiella | 25 | 26 |
| pneumoniae IAM1063 | | |
| butanediol dehydrogenase from Bacillus cereus | 27 | 28 |
| butB, butanediol dehydrogenase from Lactococcus | 29 | 30 |
| lactis | | |
| BDH1 butanediol dehydrogenase from | 54 | 55 |
| Saccharomyces cerevisiae | | |
| RdhtA, B12-indep diol dehydratase from Roseburia | 31 | 32 |
| inulinivorans | | |
| RdhtB, B12-indep diol dehydratase reactivase from | 33 | 34 |
| Roseburia inulinivorans | | |
| sadB, butanol dehydrogenase from Achromobacter | 35 | 36 |
| xylosoxidans | | |
| S. cerevisiae ILV5 | 37 | 38 |
| (acetohydroxy acid reductoisomerase) | | |
| Vibrio cholerae ketol-acid reductoisomerase | 39 | 40 |
| (KARI) | | |
| Pseudomonas aeruginosa ketol-acid | 41 | 42 |
| reductoisomerase | | |
| Pseudomonas fluorescens ketol-acid | 43 | 44 |
| reductoisomerase | | |
| Pf5.IlvC-Z4B8 mutant Pseudomonas fluorescens | 45 | 46 |
| acetohydroxy acid reductoisomerase (codon | | |
| optimized for S. cerevisiae expression) | | |
| Lactococcus lactis ilvC | 58 | 59 |
| S. cerevisiae ILV3 | 47 | 48 |
| (Dihydroxyacid dehydratase; DHAD) | | |
| Streptococcus mutans ilvD (DHAD) | 49 | 50 |
| Lactococcus lactis kivD (branched-chain α-keto | 51 | 52 |
| acid decarboxylase) | | |
| L. lactis kivD codon optimized for S. cerevisiae | 53 | 52* |
| expression | | |
| Equus caballus alcohol dehydrogenase codon | 56 | 57 |
| optimized for S. cerevisiae expression | | |
| Saccharomyces cerevisiae ALD6 | — | 60 |
| Saccharomyces cerevisiae YMR226C | — | 63 |
| Beijerinkia indica alcohol dehydrogenase | — | 74 |
| Anaerostipes caccae KARI variant K9D3 | — | 61 |
| Anaerostipes caccae KARI variant K9G9 | — | 62 |
| Saccharomyces cerevisiae AFT1 | 64 | 65 |
| Saccharomyces cerevisiae AFT2 | 66 | 67 |
| Saccharomyces cerevisiae FRA2 | 68 | 69 |
| Saccharomyces cerevisiae GRX3 | 70 | 71 |
| Saccharomyces cerevisiae CCC1 | 72 | 73 |

*The same amino acid sequence is encoded by SEQ ID NOs: 51 and 53

SEQ ID NOs: 55-59 are hybrid promoter sequences.

The invention can be more fully understood from the following detailed description which forms a part of this application.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

The present invention relates to yeast production cultures that have improved fermentation to produce butanol due to reduced sensitivity to butanol that is present in the culture medium. Butanol includes isobutanol and 1-butanol. In addition, the invention relates to methods of producing butanol using the present cultures. Butanol is useful for replacing fossil fuels, in addition to applications as solvents and/or extractants

The following definitions and abbreviations are to be use for the interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

The term “butanol” as used herein, refers to 1-butanol, isobutanol, or mixtures thereof.

The term “isobutanol biosynthetic pathway” or “isobutanol pathway” refers to an enzyme pathway to produce isobutanol from pyruvate.

The term “1-butanol biosynthetic pathway” or “1-butanol pathway” refers to an enzyme pathway to produce 1-butanol from pyruvate.

The term “low cell density” refers to a cell concentration of less than about 6×10⁵ cells/ml. For example, a culture with an OD₆₀₀ of 0.05 is a low cell density culture, based on the relationship that 1.0 OD₆₀₀ corresponds to 10⁷ cell/ml.

The term “high cell density” refers to a cell concentration of greater than about 5×10⁷ cells/ml. For example, a culture with an OD₆₀₀ of 5.0 and about 2.4 grams dry cell weight per liter (gdcw/L) is a high cell density culture, based on the relationship that 1.0 OD₆₀₀ corresponds to 10⁷ cell/ml and to 0.4 gdcw/L.

The terms “glucose utilization” and “glucose consumption” refer to the amount of glucose that a cell culture metabolizes under conditions of excess glucose. Glucose utilization rate is measured in a culture of defined cell density in a defined concentration of butanol in culture conditions as described in Example 3 herein with glucose as the carbon substrate. When alternative carbon substrates are present in media used for production, the glucose utilization rate cannot be measured in that culture, but must be measured in a separate culture with glucose as the carbon substrate.

The term “carbon substrate” or “fermentable carbon substrate” or “suitable carbon substrate” refers to a carbon source capable of being metabolized by cultures of the present invention and particularly include carbon sources selected from the group consisting of monosaccharides, oligosaccharides, and polysaccharides.

The term “fermentative production” refers to the conversion of a carbon source to a product by the metabolic activity of a microorganism, such as in this case by cultures of the present invention.

The term “viable” refers to a culture of cells (e.g., yeast cells) capable of multiplying or being cultured under the growth conditions provided herein or in a growth medium containing butanol at a concentration of at least about 2% (w/v). In some embodiments, a viable culture is capable of multiplying or being cultured under such conditions for 24 hours.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

### Improved Butanol Tolerance in High Cell Density Culture

Disclosed herein is the discovery that when yeast cells are in a high cell density culture, the cells show increased tolerance to butanol as compared to yeast cells in a low cell density culture. High cell density culture for the present purposes refers to a culture with a cell density of at least about 2.4 gram dry cell weight per liter (gdcw/L). Any culture with greater cell density than about 2.4 gdcw/L, such as one with at least about 3.8 gdcw/L or higher, including 24 gdcw/L or higher, is a high cell density culture. High cell density may be greater than about 3 gdcw/L, greater than about 5 gdcw/L, greater than about 7 gdcw/L, greater than about 10 gdcw/L, greater than about 20 gdcw/L, or greater than about 30 gdcw/L. It is envisioned that cell densities as high as about 35-40 gdcw/L may be useful. For yeast, high cell density may also be characterized as a cell concentration of greater than about 5×10⁷ cells/ml or a measured OD₆₀₀ of at least about 5. In some embodiments, the cell density can be any range of cell densities described herein, for example, from about 2.4 gdcw/L to about 40 gdcw/L, from about 2.4 gdcw/L to about 35 gdcw/L, from about 2.4 gdcw/L to about 30 gdcw/L, from about 2.4 gdcw/L to about 20 gdcw/L, from about 2.4 gdcw/L to about 10 gdcw/L, from about 2.4 gcdw/L to about 7 gdcw/L, from about 2.4 gdcw/L to about 5 gdcw/L, from about 3 gdcw/L to about 40 gdcw/L, from about 3 gdcw/L to about 35 gdcw/L, from about 3 gdcw/L to about 30 gdcw/L, from about 3 gdcw/L to about 20 gdcw/L, from about 3 gdcw/L to about 10 gdcw/L, from about 3 gcdw/L to about 7 gdcw/L, from about 3 gdcw/L to about 5 gdcw/L, from about 7 gdcw/L to about 40 gdcw/L, from about 7 gdcw/L to about 35 gdcw/L, from about 7 gdcw/L to about 30 gdcw/L, from about 7 gdcw/L to about 20 gdcw/L, or from about 7 gdcw/L to about 10 gdcw/L. For comparison, low cell density cultures have less than about 6×10⁵ cells/ml.

Increased tolerance of yeast to butanol was assessed herein by survival and/or utilization of glucose, which was found to be improved in high cell density cultures with respect to that in cultures of yeast cells at low cell density. Survival was measured by assaying the number of colony forming units (CFU), which is a measure of live cells. Yeast cultures with high cell densities in the presence of 1% butanol were found to have survival rates of at least about 40%, 50%, 60%, 70%, 75%, 80%, and up to 100% as compared to survival rate of a control culture without butanol under conditions assayed herein. Survival rate varies and depends on multiple factors including concentration of butanol, specific butanol isomer, and strain of yeast. In contrast, survival of low cell density cultures, having 100-fold fewer cells, was found in 1% butanol to be at rates up to 13% as compared to survival rate of a control culture without butanol.

The effect of yeast cell density on tolerance to alcohols was found not to be one response generalized to the presence of alcohols. The effect of cell density on butanol tolerance was different from the effect of cell density in the presence of the most widely produced alcohol, ethanol. In fact, as shown herein (see Example 1), the opposite effect was observed in ethanol, with low cell density cultures showing higher survival than high cell density cultures.

High cell density cultures having 3.8 gdcw/L and 24 gdcw/L were found herein to utilize glucose in 1.5% isobutanol at a rate of at least about 1 gram per gdcw per hour. In 1% isobutanol the calculated rate was at least about 1.4 gram per gdcw per hour. In 2% isobutanol the rate was at least about 0.5 gram per gdcw per hour, in 2.5% isobutanol the rate was at least about 0.4 gram per gdcw per hour and in 3% isobutanol the rate was at least about 0.2 gram per gdcw per hour.

Thus, provided herein are butanol production cultures having a high cell density of at least about 2.4 gdcw/L so that higher tolerance to butanol is achieved. High cell density cultures may be at least about 2.4 gdcw/L, 3.8 gdcw/L, or 24 gdcw/L, or higher. Butanol concentration in the production culture is at least about 1%, and may be at least about 1.5%, 2.0%, 2.5%, or 3.0% (w/v). In some embodiments, the butanol concentration is at least about 2.0%. In some embodiments, the butanol concentration is any range of the butanol concentrations disclosed herein, for example, from about 1% to about 3%, from about 1.5% to about 3%, from about 2% to about 3%, from about 1.5% to about 2.5%, or from about 2% to about 3% (w/v). Butanol can be isobutanol or 1-butanol. In the present production culture the glucose utilization rate is at least about 0.2 grams per gram of dry cell weight per hour (g/gdcw/h). The glucose utilization rate may be higher, such as at least about 0.3, 0.4, 0.5, 0.6, 1, 1.5, 2.4, or 3 gram per gdcw per hour. In some embodiments, the butanol concentration is at least about 2% w/v and the glucose utilization rate is at least about 0.5, 0.6, 1, 1.5, 2.4, or 3 gram per gdcw per hour. In some embodiments, the glucose utilization rate is at least about 0.5 gram per gdcw per hour. In some embodiments, the glucose utilization rate can be any range of the glucose utilization rates described herein, for example, from about 0.3 g/gdcw/h to about 3 g/gdcw/h, from about 0.3 g/gdcw/h to about 2.4 g/gdcw/h, from about 0.3 g/gdcw/h to about 1.5 g/gdcw/h, from about 0.3 g/gdcw/h to about 1 g/gdcw/h, from about 0.3 g/gdcw/h to about 0.6 g/gdcw/h, from about 0.3 g/gdcw/h to about 0.5 g/gdcw/h, from about 0.3 g/gdcw/h to about 0.4 g/gdcw/h, from about 0.5 g/gdcw/h to about 3 g/gdcw/h, from about 0.5 g/gdcw/h to about 2.4 g/gdcw/h, from about 0.5 g/gdcw/h to about 1.5 g/gdcw/h, from about 0.5 g/gdcw/h to about 1 g/gdcw/h, from about 0.5 g/gdcw/h to about 0.6 g/gdcw/h, from about 1 g/gdcw/h to about 3 g/gdcw/h, from about 1 g/gdcw/h to about 2.4 g/gdcw/h, or from about 1 g/gdcw/h to about 1.5 g/gdcw/h. In some embodiments, the glucose concentration is at least about 2.0% w/v and the glucose utilization rate is a range from about 0.5 g/gdcw/h to about 3 g/gdcw/h, from about 0.5 g/gdcw/h to about 2.4 g/gdcw/h, from about 0.5 g/gdcw/h to about 1.5 g/gdcw/h, from about 0.5 g/gdcw/h to about 1 g/gdcw/h, from about 0.5 g/gdcw/h to about 0.6 g/gdcw/h, from about 0.5 g/gdcw/h to about 3 g/gdcw/h, from about 0.5 g/gdcw/h to about 2.4 g/gdcw/h, from about 0.5 g/gdcw/h to about 1.5 g/gdcw/h, from about 0.5 g/gdcw/h to about 1 g/gdcw/h, from about 0.5 g/gdcw/h to about 0.6 g/gdcw/h, from about 1 g/gdcw/h to about 3 g/gdcw/h, from about 1 g/gdcw/h to about 2.4 g/gdcw/h, or from about 1 g/gdcw/h to about 1.5 g/gdcw/h. The glucose utilization rate achieved will depend on the concentration of butanol and the specific type of butanol in the culture medium. In general the rate will decrease with increasing butanol concentration. In general, yeast cells have similar response to isobutanol and 1-butanol, with less sensitivity to 2-butanol.

The glucose utilization rate is typically determined at a temperature of about 30° C. to about 45° C. The glucose utilization rate can also be determined at a temperature of about 30° C. to about 37° C. In some embodiments, cultures provided herein have a glucose utilization rate of greater than about 0.5 gram per gram of dry cell weight per hour at a temperature between about 30° C. to about 45° C. In some embodiments, cultures provided herein have a glucose utilization rate of greater than about 0.5 gram per gram of dry cell weight per hour at a temperature between about 30° C. to about 37° C. In some embodiments, cultures provided herein have a glucose utilization rate of greater than about 0.5 gram per gram of dry cell weight per hour at a temperature between about 30° C. to about 32° C. In some embodiments, the glucose utilization rate is determined for a culture that has been in contact with a medium comprising butanol at a concentration of at least about 2% (w/v) for at least about 6 hours. In some embodiments, cultures provided herein have a glucose utilization rate of greater than about 0.5 gram per gram of dry cell weight per hour for a culture that has been in contact with a medium comprising butanol at a concentration of at least about 2% (w/v) for at least about 6 hours.

In some embodiments, the cultures described herein are viable cultures.

### Preparation of High Cell Density Production Cultures

The present high cell density butanol production cultures may be prepared by any method that provides a cell density of at least about 2.4 gdcw/L. Cultures with cell densities of, for example, 2.4 gdcw/L, 2.7 gdcw/L, 2.8 gdcw/L, 3.8 gdcw/L or 24 gdcw/L or higher may be prepared as high cell density cultures. In some embodiments, the cell density can be any range of cell densities described herein, for example, from about 2.4 gdcw/L to about 40 gdcw/L, from about 2.4 gdcw/L to about 35 gdcw/L, from about 2.4 gdcw/L to about 30 gdcw/L, from about 2.4 gdcw/L to about 20 gdcw/L, from about 2.4 gdcw/L to about 10 gdcw/L, from about 2.4 gcdw/L to about 7 gdcw/L, from about 2.4 gdcw/L to about 5 gdcw/L, from about 3 gdcw/L to about 40 gdcw/L, from about 3 gdcw/L to about 35 gdcw/L, from about 3 gdcw/L to about 30 gdcw/L, from about 3 gdcw/L to about 20 gdcw/L, from about 3 gdcw/L to about 10 gdcw/L, from about 3 gcdw/L to about 7 gdcw/L, from about 3 gdcw/L to about 5 gdcw/L, from about 7 gdcw/L to about 40 gdcw/L, from about 7 gdcw/L to about 35 gdcw/L, from about 7 gdcw/L to about 30 gdcw/L, from about 7 gdcw/L to about 20 gdcw/L, or from about 7 gdcw/L to about 10 gdcw/L. For example, in one method yeast cells that are capable of producing butanol are grown in an aerated culture which minimizes butanol production. For example, crabtree-positive yeast cells may be grown with high aeration and in low glucose concentration to maximize respiration and cell mass production, as known in the art, rather than butanol production. Typically the glucose concentration is kept to less than about 0.2 g/L. The aerated culture can grow to a high cell density and then be used as the present production culture. Alternatively, yeast cells that are capable of producing butanol may be grown and concentrated to produce a high cell density culture.

In addition, expression of the butanol biosynthetic pathway may be regulated such that it is minimally active during growth of yeast cells to high cell density. One or more genes of the pathway may be expressed from a promoter that may be controlled by growth conditions or media components to regulate their expression. In this culture cells may grow to high cell density to be used as a production culture or to be used as a seed culture for starting a high cell density production culture.

Cultures for production of high cell density cultures are grown in media using conditions as described below in sections on Fermentation media and Culture conditions, except that glucose can be limited to about 2 g/L and aerobic conditions can be as described above for maximizing respiratory growth.

### Butanol Producing Yeast Cells

The present high cell density production cultures may be cultures of any yeast that produces butanol. The yeast may be crabtree positive or crabtree negative. Crabtree-positive yeast cells demonstrate the crabtree effect, which is a phenomenon whereby cellular respiration is inhibited when a high concentration of glucose is added to aerobic culture medium. Suitable yeasts include, but are not limited to, Saccharomyces, Schizosaccharomyces, Hansenula, Candida, Kluyveromyces, Yarrowia, Issatchenkia and Pichia. Suitable strains include, but are not limited to, Saccharomyces cerevisiae, Schizosaccharomyces pombe, Kluyveromyces lactis, Kluyveromyces thermotolerans, Candida glabrata, Candida albicans, Pichia stipitis, Issatchenkia orientalis and Yarrowia lipolytica.

Any of these yeasts that are engineered or otherwise able to produce butanol may be used in the present cultures. A biosynthetic pathway for production of isobutanol, 1-butanol, or 2-butanol is constructed in the yeast cell so that it produces butanol. The pathway genes may include endogenous genes and/or heterologous genes.

Standard recombinant DNA and molecular cloning techniques for recombinant host cells are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Press, Cold Spring Harbor, N.Y. (1984); and by Ausubel, F. M. et al., Current Protocols in Molecular Biology, published by Greene Publishing Assoc. and Wiley-Interscience (1987). Other molecular tools and techniques are known in the art and include splicing by overlapping extension polymerase chain reaction (PCR) (Yu, et al. (2004) Fungal Genet. Biol. 41:973-981), positive selection for mutations at the URA3 locus of Saccharomyces cerevisiae (Boeke, J. D. et al. (1984) Mol. Gen. Genet. 197, 345-346; M A Romanos, et al. Nucleic Acids Res. 1991 January 11; 19(1): 187), the cre-lox site-specific recombination system as well as mutant lox sites and FLP substrate mutations (Sauer, B. (1987) Mol Cell Biol 7: 2087-2096; Senecoff, et al. (1988) Journal of Molecular Biology, Volume 201, Issue 2, Pages 405-421; Albert, et al. (1995) The Plant Journal. Volume 7, Issue 4, pages 649-659), “seamless” gene deletion (Akada, et al. (2006) Yeast; 23(5):399-405), and gap repair methodology (Ma et al., Genetics 58:201-216; 1981).

Methods for gene expression in yeasts are known in the art as described, for example, in Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). For example, a chimeric gene for expression may be constructed by operably linking a promoter and terminator to a coding region. Promoters that may used include, for example, constitutive promoters FBA1, TDH3, ADH1, and GPM1, and the inducible promoters GAL1, GAL10, and CUP1. Other yeast promoters include hybrid promoters UAS(PGK1)-FBA1p (SEQ ID NO: 55), UAS(PGK1)-ENO2p (SEQ ID NO: 56), UAS(FBA1)-PDC1p (SEQ ID NO: 57), UAS(PGK1)-PDC1p (SEQ ID NO: 58), and UAS(PGK)-OLE1p (SEQ ID NO: 59). Suitable transcriptional terminators that may be used in a chimeric gene construct for expression include, but are not limited to FBA1t, TDH3t, GPM1t, ERG10t, GAL1t, CYC1t, and ADH1t.

Suitable promoters, transcriptional terminators, and coding regions may be cloned into E. coli-yeast shuttle vectors, and transformed into yeast cells. These vectors allow for propagation in both E. coli and yeast strains. Typically the vector contains a selectable marker and sequences allowing autonomous replication or chromosomal integration in the desired host. Typically used plasmids in yeast are shuttle vectors pRS423, pRS424, pRS425, and pRS426 (American Type Culture Collection, Rockville, Md.), which contain an E. coli replication origin (e.g., pMB1), a yeast 2μ origin of replication, and a marker for nutritional selection. The selection markers for these four vectors are HIS3 (vector pRS423), TRP1 (vector pRS424), LEU2 (vector pRS425) and URA3 (vector pRS426). Construction of expression vectors with a chimeric gene for expression may be performed by either standard molecular cloning techniques in E. coli or by the gap repair recombination method in yeast.

The gap repair cloning approach takes advantage of the highly efficient homologous recombination in yeast. Typically, a yeast vector DNA is digested (e.g., in its multiple cloning site) to create a “gap” in its sequence. A number of insert DNAs of interest are generated that contain a ≧21 bp sequence at both the 5′ and the 3′ ends that sequentially overlap with each other, and with the 5′ and 3′ terminus of the vector DNA. For example, to construct a yeast expression vector for “Gene X′, a yeast promoter and a yeast terminator are selected for the expression cassette. The promoter and terminator are amplified from the yeast genomic DNA, and Gene X is either PCR amplified from its source organism or obtained from a cloning vector comprising Gene X sequence. There is at least a 21 bp overlapping sequence between the 5′ end of the linearized vector and the promoter sequence, between the promoter and Gene X, between Gene X and the terminator sequence, and between the terminator and the 3′ end of the linearized vector. The “gapped” vector and the insert DNAs are then co-transformed into a yeast strain and plated on the medium containing the appropriate compound mixtures that allow complementation of the nutritional selection markers on the plasmids. The presence of correct insert combinations can be confirmed by PCR mapping using plasmid DNA prepared from the selected cells. The plasmid DNA isolated from yeast (usually low in concentration) can then be transformed into an E. coli strain, e.g. TOP10, followed by mini preps and restriction mapping to further verify the plasmid construct. Finally the construct can be verified by DNA sequence analysis.

Like the gap repair technique, integration into the yeast genome also takes advantage of the homologous recombination system in yeast. Typically, a cassette containing a coding region plus control elements (promoter and terminator) and auxotrophic marker is PCR-amplified with a high-fidelity DNA polymerase using primers that hybridize to the cassette and contain 40-70 base pairs of sequence homology to the regions 5′ and 3′ of the genomic area where insertion is desired. The PCR product is then transformed into yeast and plated on medium containing the appropriate compound mixtures that allow selection for the integrated auxotrophic marker. For example, to integrate “Gene X” into chromosomal location “Y”, the promoter-coding regionX-terminator construct is PCR amplified from a plasmid DNA construct and joined to an autotrophic marker (such as URA3) by either SOE PCR (Horton et al. (1989) Gene 77:61-68) or by common restriction digests and cloning. The full cassette, containing the promoter-coding regionX-terminator-URA3 region, is PCR amplified with primer sequences that contain 40-70 bp of homology to the regions 5′ and 3′ of location “Y” on the yeast chromosome. The PCR product is transformed into yeast and selected on growth media lacking uracil. Transformants can be verified either by colony PCR or by direct sequencing of chromosomal DNA.

### Butanol Production Pathways

Suitable pathways for production of butanol are known in the art, and certain suitable pathways are described herein. In some embodiments, the butanol production pathway comprises at least one gene that is heterologous to the host cell. In some embodiments, the butanol biosynthetic pathway comprises more than one gene that is heterologous to the host cell. In some embodiments, the butanol biosynthetic pathway comprises heterologous genes encoding polypeptides corresponding to every step of a biosynthetic pathway. As is known in the art, sequences may be codon optimized for expression in a host cell.

Genes and polypeptides that can be used for substrate to product conversions described herein as well as methods of identifying such genes and polypeptides, are described herein and/or in the art, for example, for isobutanol, in U.S. Pat. No. 7,851,188.

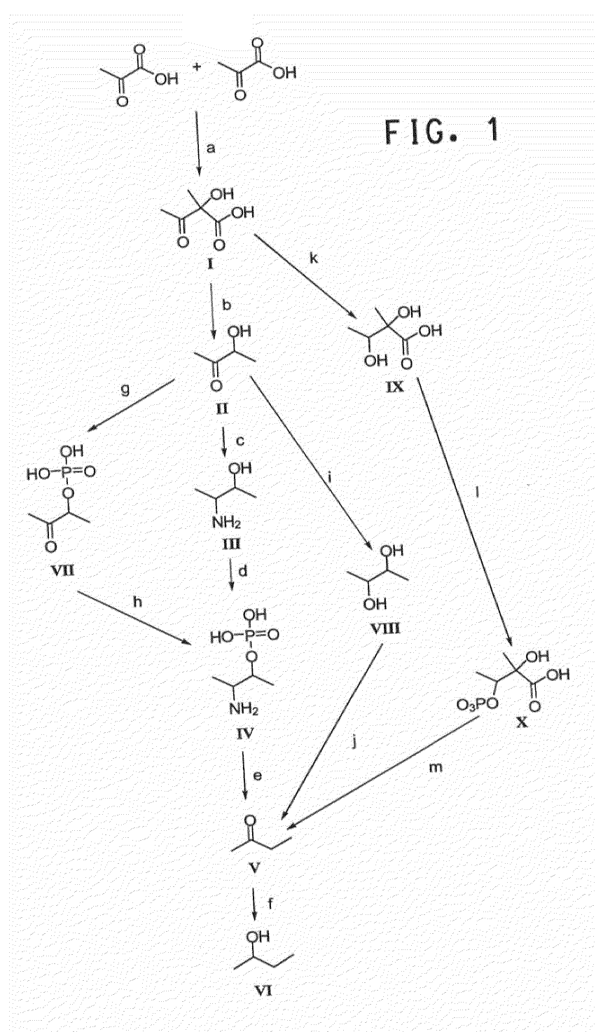

Biosynthetic pathways for production of 2-butanol that may be engineered in the present cells are disclosed in the art, for example, in US Patent Application Publications US20070292927A1 and US20070259410A1, which are herein incorporated by reference. A diagram of the disclosed 2-butanol biosynthetic pathways is provided in FIG. 1. For example, the pathway in US20070292927A1 includes the following conversion steps:
pyruvate to acetolactate (FIG. 1 step a) as catalyzed for example by acetolactate synthase;
acetolactate to acetoin (FIG. 1 step b) as catalyzed for example by acetolactate decarboxylase;
acetoin to 2,3-butanediol (FIG. 1 step i) as catalyzed for example by butanediol dehydrogenase;
2,3-butanediol to 2-butanone (FIG. 1 step j) as catalyzed for example by diol dehydratase or glycerol dehydratase; and
2-butanone to 2-butanol (FIG. 1 step f) as catalyzed for example by butanol dehydrogenase.

As disclosed in US Patent Application Publication No. US 2009-0305363, for production of the 2,3-butanediol intermediate in yeast pdc− host cells, acetolactate synthase may be expressed in the cytosol. Acetolactate synthase enzymes, which also may be called acetohydroxy acid synthase, belong to EC 2.2.1.6 (switched from 4.1.3.18 in 2002), are well-known, and they participate in the biosynthetic pathway for the proteinogenic amino acids leucine and valine, as well as in the pathway for fermentative production of 2,3-butanediol from acetoin in a number of organisms. The skilled person will appreciate that polypeptides having acetolactate synthase activity isolated from a variety of sources may be used in the present cells. Acetolactate synthase (Als) enzyme activities that have substrate preference for pyruvate over ketobutyrate are of particular utility, such as those encoded by alsS of Bacillus and budB of Klebsiella (Gollop et al., J. Bacteriol. 172(6):3444-3449 (1990); Holtzclaw et al., J. Bacteriol. 121(3):917-922 (1975)). Als from Bacillus subtilis (DNA: SEQ ID NO:3; protein: SEQ ID NO:4), from Klebsiella pneumoniae (DNA: SEQ ID NO:1; protein: SEQ ID NO:2), and from Lactococcus lactis (DNA: SEQ ID NO:5; protein: SEQ ID NO:6) are provided herein.

Additional Als coding regions and encoded proteins that may be used include those from Staphylococcus aureus (DNA: SEQ ID NO:7; protein: SEQ ID NO:8), Listeria monocytogenes (DNA: SEQ ID NO:9; protein: SEQ ID NO:10), Streptococcus mutans (DNA: SEQ ID NO:11; protein: SEQ ID NO:12), Streptococcus thermophilus (DNA: SEQ ID NO:13; protein: SEQ ID NO:14), Vibrio angustum (DNA: SEQ ID NO:15; protein: SEQ ID NO:16), and Bacillus cereus (DNA: SEQ ID NO:17; protein: SEQ ID NO:18). Any Als gene that encodes an acetolactate synthase having at least about 80-85%, 85%-90%, 90%-95%, or at least about 96%, 97%, or 98% sequence identity to any of those with SEQ ID NOs:2, 4, 6, 8, 10, 12, 14, 16, or 18 that converts pyruvate to acetolactate may be used. Identities are based on the Clustal W method of alignment using the default parameters of GAP PENALTY=10, GAP LENGTH PENALTY=0.1, and Gonnet 250 series of protein weight matrix.

Additionally, US Patent Application Publication No. 20090305363 provides a phylogenetic tree depicting acetolactate synthases that are the 100 closest neighbors of the B. subtilis AlsS sequence, any of which may be used. Additional Als sequences that may be used in the present strains may be identified in the literature and in bioinformatics databases as is well known to the skilled person. Identification of coding and/or protein sequences using bioinformatics is typically through BLAST (described above) searching of publicly available databases with known Als encoding sequences or encoded amino acid sequences, such as those provided herein. Identities are based on the Clustal W method of alignment as specified above. Additionally, the sequences described herein or those recited in the art may be used to identify other homologs in nature as described above.

Cytosolic expression of acetolactate synthase is achieved by transforming with a gene comprising a sequence encoding an acetolactate synthase protein, with no mitochondrial targeting signal sequence. Methods for gene expression in yeasts are known in the art (see for example Methods in Enzymology, Volume 194, Guide to Yeast Genetics and Molecular and Cell Biology (Part A, 2004, Christine Guthrie and Gerald R. Fink (Eds.), Elsevier Academic Press, San Diego, Calif.). Expression using chimeric genes (including coding regions with operably linked promoters and terminators), vectors, cloning methods, and integration methods are as described above.

Conversion of acetolactate to acetoin is by an acetolactate decarboxylase enzyme, known as EC 4.1.1.5 which is available, for example, from Bacillus subtilis (DNA: SEQ ID NO:21; Protein: SEQ ID NO:22), Klebsiella terrigena (DNA: SEQ ID NO:23, Protein: SEQ ID NO:24) and Klebsiella pneumoniae (DNA: SEQ ID NO:19, protein: SEQ ID NO:20). Any gene that encodes an acetolactate decarboxylase having at least about 80-85%, 85%-90%, 90%-95%, or at least about 96%, 97%, or 98% sequence identity to any of those with SEQ ID NOs:20, 22, or 24 that converts acetolactate to acetoin may be used.

Conversion of acetoin to 2,3-butanediol is by a butanediol dehydrogenase enzyme, also known as acetoin reductase. Butanediol dehydrogenase enzymes may have specificity for production of (R)- or (S)-stereochemistry in the alcohol product. (S)-specific butanediol dehydrogenases are known as EC 1.1.1.76 and are available, for example, from Klebsiella pneumoniae (DNA: SEQ ID NO:25; protein: SEQ ID NO:26). (R)-specific butanediol dehydrogenases are known as EC 1.1.1.4 and are available, for example, from Bacillus cereus (DNA: SEQ ID NO:27, protein: SEQ ID NO:28), Lactococcus lactis (DNA: SEQ ID NO:29, protein: SEQ ID NO:30), and Saccharomyces cerevisiae (BDH1; DNA: SEQ ID NO:54, protein: SEQ ID NO:55). Any gene that encodes a butanediol dehydrogenase having at least about 80-85%, 85%-90%, 90%-95%, or at least about 98% sequence identity to any of those with SEQ ID NOs:26, 28, 30 or 55 that converts acetoin to 2,3-butanediol may be used.

Diol dehydratases, also known as butanediol dehydratases, which utilize the cofactor adenosyl cobalamin (vitamin B12) are known as EC 4.2.1.28. Glycerol dehydratases that also utilize the cofactor adenosyl cobalamin are known as EC 4.2.1.30. Diol and glycerol dehydratases have three subunits that are required for activity. Provided in US 20070292927A1 are examples of sequences of the three subunits of many diol and glycerol dehydratases that may be used in a 2-butanone or 2-butanol pathway in the present cells, as well as the preparation and use of a Hidden Markov Model to identify additional diol and dehydratase enzymes that may be used.

Butanol dehydrogenases are a subset of a broad family of alcohol dehydrogenases and may be NAD⁺- or NADP⁺-dependent. The NAD-dependent enzymes are known as EC 1.1.1.1 and the NADP-dependent enzymes are known as EC 1.1.1.2. Provided in US 20070292927A1 are examples of sequences of butanol dehydrogenases that may be used in the disclosed 2-butanol biosynthetic pathway in the present cells.

Described in US Patent Application Publication US 20090155870 A1, which is herein incorporated by reference, are construction of chimeric genes and genetic engineering of yeast for 2-butanol production using the US 20070292927A1 disclosed biosynthetic pathway. Further description for gene construction and expression is above and in the Examples herein.

The use in this pathway in yeast of the butanediol dehydratase from Roseburia inulinivorans, RdhtA, (protein SEQ ID NO:32, coding region SEQ ID NO:31) is disclosed in commonly owned and co-pending US Patent Application Publication US 20090155870 A1. This enzyme is used in conjunction with the butanediol dehydratase reactivase from Roseburia inulinivorans, RdhtB, (protein SEQ ID NO:34, coding region SEQ ID NO: 33). This butanediol dehydratase is desired in many hosts because it does not require coenzyme B₁₂. Another B₁₂-dependent diol dehydratase that may be used is one from Klebsiella pneumoniae, having three subunits: pduC, pduD, and pduE, that is disclosed in WO2009046370.

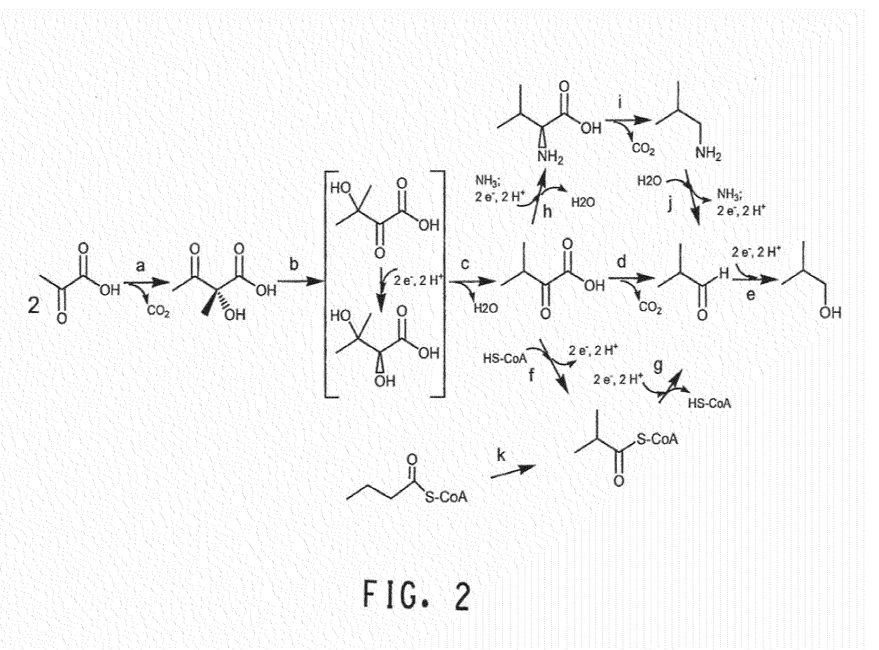

Useful for the last step of converting 2-butanone to 2-butanol in all pathways of FIG. 2 is a butanol dehydrogenase isolated from an environmental isolate of a bacterium identified as Achromobacter xylosoxidans that is disclosed in US Patent Application Publication No. 20090269823 (DNA: SEQ ID NO:35, protein SEQ ID NO:36), which is herein incorporated by reference.

Genes and their expression for other pathways of FIG. 2 are disclosed in US20070259410A1. Additional sequences that may be used to express the disclosed enzyme activities in the present strains may be identified in the literature and in bioinformatics databases as is well known to the skilled person. Identification of coding and/or protein sequences using bioinformatics is typically done using sequence analysis software such as BLAST (BLASTP, BLASTN, BLASTX (Altschul et al., J. Mol. Biol., 215:403-410 (1990)) and searching of publicly available databases with known encoding sequences or encoded amino acid sequences, such as those provided herein. Identities are based on the Clustal W method of alignment (described by Higgins and Sharp, CABIOS. 5:151-153 (1989); Higgins, D. G. et al., Comput. Appl. Biosci. 8:189-191 (1992)) and found in the MegAlign™ v6.1 program of the LASERGENE bioinformatics computing suite (DNASTAR Inc.). Default parameters for multiple alignment (GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB).

Additionally, the sequences described herein or those recited in the art may be used to identify other homologs in nature. For example each of the encoding nucleic acid fragments described herein may be used to isolate genes encoding homologous proteins. Isolation of homologous genes using sequence-dependent protocols is well known in the art. Examples of sequence-dependent protocols include, but are not limited to: 1) methods of nucleic acid hybridization; 2) methods of DNA and RNA amplification, as exemplified by various uses of nucleic acid amplification technologies [e.g., polymerase chain reaction (PCR), Mullis et al., U.S. Pat. No. 4,683,202; ligase chain reaction (LCR), Tabor, S. et al., Proc. Acad. Sci. USA 82:1074 (1985); or strand displacement amplification (SDA), Walker, et al., Proc. Natl. Acad. Sci. U.S.A., 89:392 (1992)]; and 3) methods of library construction and screening by complementation.

Biosynthetic pathways for production of isobutanol that may be engineered in the present cells are disclosed in the art, for example, in US Patent Application Publication US 20070092957 A1, which is herein incorporated by reference. A diagram of the disclosed isobutanol biosynthetic pathways is provided in FIG. 2. As described in US 20070092957 A1, steps in an example isobutanol biosynthetic pathway include conversion of:
pyruvate to acetolactate (FIG. 2 pathway step a) as catalyzed for example by acetolactate synthase
acetolactate to 2,3-dihydroxyisovalerate (FIG. 2 pathway step b) as catalyzed for example by acetohydroxy acid isomeroreductase, also called ketol-acid reductoisomerase;
2,3-dihydroxyisovalerate to α-ketoisovalerate (FIG. 2 pathway step c) as catalyzed for example by acetohydroxy acid dehydratase, also called dihydroxy-acid dehydratase;
α-ketoisovalerate to isobutyraldehyde (FIG. 2 pathway step d) as catalyzed for example by branched-chain α-keto acid decarboxylase; and
isobutyraldehyde to isobutanol (FIG. 2 pathway step e) as catalyzed for example by branched-chain alcohol dehydrogenase.

Acetolactate synthase was described above for the 2,3-butanediol pathway.

Acetohydroxy acid isomeroreductase, also called ketol-acid reductoisomerase (KARI) may naturally use NADPH (reduced nicotinamide adenine dinucleotide phosphate) as an electron donor. KARIs include those known by the EC number 1.1.1.86. Examples of sequences of KARI enzymes and their coding regions are provided in US20070092957 A1, including ILV5 from Saccharomyces cerevisiae (DNA: SEQ ID NO:37; protein SEQ ID NO:38). Ketol-acid reductoisomerase (KARI) enzymes are described in U.S. Patent Appl. Pub. Nos. 20080261230 A1, 20090163376 A1, 20100197519 A1, and PCT Appl. Pub. No. WO/2011/041415, all herein incorporated by reference. Examples of KARIs disclosed therein are those from Vibrio cholerae (DNA: SEQ ID NO:39; protein SEQ ID NO:40), Pseudomonas aeruginosa PAO1, (DNA: SEQ ID NO:41; protein SEQ ID NO:42), and Pseudomonas fluorescens PF5 (DNA: SEQ ID NO:43; protein SEQ ID NO:44) and Pf5.IlvC-Z4B8 mutant Pseudomonas fluorescens acetohydroxy acid reductoisomerase (DNA: SEQ ID NO:45; protein SEQ ID NO:46). Another KARI is encoded by the ilvC gene of Lactococcus lactis (DNA: SEQ ID NO:58; protein SEQ ID NO:59). KARIs also include Anaerostipes caccae KARI variants “K9G9” and “K9D3” (SEQ ID NOs: 62 and 61, respectively).

Acetohydroxy acid dehydratases, also called dihydroxy acid dehydratases (DHAD), are known by the EC number 4.2.1.9. Examples of sequences of DHAD enzymes and their coding regions are provided in U.S. Pat. No. 7,851,188 dihydroxyacid dehydratases (DHADs), including ILV3 of Saccharomyces cerevisiae (DNA: SEQ ID NO:47; protein SEQ ID NO:48). Additional [2Fe-2S]²⁺ DHAD sequences such as the Streptococcus mutans DHAD (DNA: SEQ ID NO:49; protein SEQ ID NO:50) and a method for identifying [2Fe-2S]²⁺ DHAD enzymes that may be used to obtain additional DHAD sequences that may be used are disclosed in co-pending US Patent Application Publication No. 20100081154, which is herein incorporated by reference.

Branched-chain α-keto acid decarboxylases (KivD) are known by the EC number 4.1.1.72. Examples of sequences of branched-chain α-keto acid decarboxylase enzymes and their coding regions are provided in US20070092957 A1, including Lactococcus lactis KivD (DNA: SEQ ID NO:51; codon optimized for expression in S. cerevisiae SEQ ID NO:53; protein SEQ ID NO:52). Additional branched-chain α-keto acid decarboxylases include one from Bacillus subtilis with coding sequence optimized for expression in S. cerevisiae (DNA: SEQ ID NO:54; protein SEQ ID NO:55), and others readily identified by one skilled in the art using bioinformatics as described above.

Branched-chain alcohol dehydrogenases are known by the EC number 1.1.1.265, but may also be classified under other alcohol dehydrogenases (specifically, EC 1.1.1.1 or 1.1.1.2). These enzymes utilize NADH (reduced nicotinamide adenine dinucleotide) and/or NADPH as electron donor and examples of sequences of branched-chain alcohol dehydrogenase enzymes and their coding regions are provided in US20070092957 A1.

U.S. Patent Appl. Publ. No. 20090269823 A1 describes SadB (DNA: SEQ ID NO:35, protein SEQ ID NO:36), an alcohol dehydrogenase (ADH) from Achromobacter xylosoxidans. Alcohol dehydrogenases also include horse liver ADH (HADH; codon optimized for expression in S. cerevisiae; DNA: SEQ ID NO:56; protein SEQ ID NO:57) and Beijerinkia indica ADH (protein SEQ ID NO: 74) as well as others readily identified by one skilled in the art using bioinformatics as described above.

Genes that may be used for expression of enzymes for two additional isobutanol pathways are described in US 20070092957 A1. Additional genes that may be used in all three pathways can be identified by one skilled in the art as described above.

Additionally described in US 20070092957 A1 are construction of chimeric genes and genetic engineering of yeast, exemplified by Saccharomyces cerevisiae, for isobutanol production using the disclosed biosynthetic pathways. Further description for gene construction and expression is above and in the Examples herein.

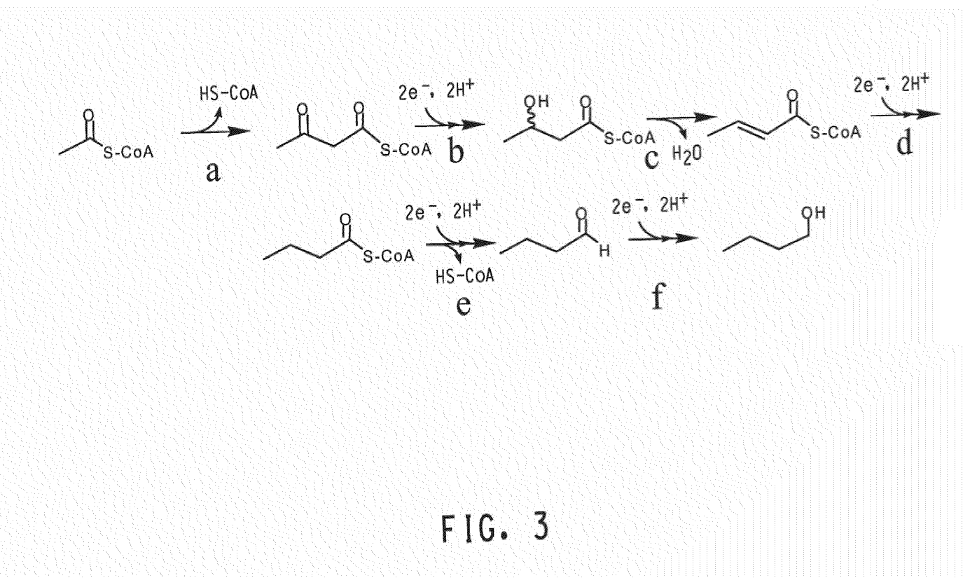

A biosynthetic pathway for production of 1-butanol that may be engineered in the present cells is disclosed in co-pending US Patent Application Publication US 20080182308A1, which is herein incorporated by reference. A diagram of the disclosed 1-butanol biosynthetic pathway is provided in FIG. 3. As described in US 20080182308A1, steps in the disclosed 1-butanol biosynthetic pathway include conversion of:
acetyl-CoA to acetoacetyl-CoA (FIG. 3 pathway step a), as catalyzed for example by acetyl-CoA acetyltransferase;
acetoacetyl-CoA to 3-hydroxybutyryl-CoA (FIG. 3 pathway step b), as catalyzed for example by 3-hydroxybutyryl-CoA dehydrogenase;
3-hydroxybutyryl-CoA to crotonyl-CoA (FIG. 3 pathway step c), as catalyzed for example by crotonase;
crotonyl-CoA to butyryl-CoA (FIG. 3 pathway step d), as catalyzed for example by butyryl-CoA dehydrogenase;
butyryl-CoA to butyraldehyde (FIG. 3 pathway step e), as catalyzed for example by butyraldehyde dehydrogenase; and
butyraldehyde to 1-butanol (FIG. 3 pathway step f), as catalyzed for example by butanol dehydrogenase.

Genes that may be used for expression of these enzymes are described in US 20080182308A1, and additional genes that may be used can be identified by one skilled in the art as described above. Methods for expression of these genes in yeast are described in US 20080182308A1 as well as herein above.

In some embodiments, the butanol biosynthetic pathway comprises at least one gene, at least two genes, at least three genes, at least four genes, or at least 5 genes that is/are heterologous to the yeast cell. In embodiments, each substrate to product conversion of a butanol biosynthetic pathway in a recombinant host cell is catalyzed by a heterologous polypeptide. In embodiments, the butanol biosynthetic pathway is an isobutanol biosynthetic pathway and the polypeptide catalyzing the substrate to product conversions of acetolactate to 2,3-dihydroxyisovalerate and/or the polypeptide catalyzing the substrate to product conversion of isobutyraldehyde to isobutanol are capable of utilizing NADH as a cofactor.

It will be appreciated that host cells comprising a butanol biosynthetic pathway such as an isobutanol biosynthetic pathway as provided herein may further comprise one or more additional modifications. U.S. Appl. Pub. No. 20090305363 (incorporated by reference) discloses increased conversion of pyruvate to acetolactate by engineering yeast for expression of a cytosol-localized acetolactate synthase and substantial elimination of pyruvate decarboxylase activity. Modifications to reduce glycerol-3-phosphate dehydrogenase activity and/or disruption in at least one gene encoding a polypeptide having pyruvate decarboxylase activity or a disruption in at least one gene encoding a regulatory element controlling pyruvate decarboxylase gene expression as described in U.S. Patent Appl. Pub. No. 20090305363 (incorporated herein by reference), modifications to a host cell that provide for increased carbon flux through an Entner-Doudoroff Pathway or reducing equivalents balance as described in U.S. Patent Appl. Pub. No. 20100120105 (incorporated herein by reference). Other modifications include integration of at least one polynucleotide encoding a polypeptide that catalyzes a step in a pyruvate-utilizing biosynthetic pathway. Other modifications include at least one deletion, mutation, and/or substitution in an endogenous polynucleotide encoding a polypeptide having acetolactate reductase activity. In embodiments, the polypeptide having acetolactate reductase activity is YMR226c (SEQ ID NO: 63) of Saccharomyces cerevisae or a homolog thereof. Additional modifications include a deletion, mutation, and/or substitution in an endogenous polynucleotide encoding a polypeptide having aldehyde dehydrogenase and/or aldehyde oxidase activity. In embodiments, the polypeptide having aldehyde dehydrogenase activity is ALD6 (SEQ ID NO: 60) from Saccharomyces cerevisiae or a homolog thereof. A genetic modification which has the effect of reducing glucose repression wherein the yeast production host cell is pdc− is described in U.S. Appl. Publication No. 20110124060, incorporated herein by reference.

Recombinant host cells may further comprise (a) at least one heterologous polynucleotide encoding a polypeptide having dihydroxy-acid dehydratase activity; and (b)(i) at least one deletion, mutation, and/or substitution in an endogenous gene encoding a polypeptide affecting Fe—S cluster biosynthesis; and/or (ii) at least one heterologous polynucleotide encoding a polypeptide affecting Fe—S cluster biosynthesis. In embodiments, the polypeptide affecting Fe—S cluster biosynthesis is encoded by AFT1 (nucleic acid SEQ ID NO: 64, amino acid SEQ ID NO: 65), AFT2 (SEQ ID NOs: 66 and 67), FRA2 (SEQ ID NOs: 68 and 69), GRX3 (SEQ ID NOs: 70 and 71), or CCC1 (SEQ ID NOs: 72 and 73). In embodiments, the polypeptide affecting Fe—S cluster biosynthesis is constitutive mutant AFT1 L99A, AFT1 L102A, AFT1 C291F, or AFT1 C293F.

### Fermentation Media

High cell density production cultures disclosed herein are maintained in culture medium that supports metabolism for production of butanol. The culture medium can also provide the culture viability for production of butanol. Typically media used for the present invention may contain at least about 2 g/L glucose or an equivalent amount of carbon substrates. Carbon substrates may include but are not limited to monosaccharides such as fructose, oligosaccharides such as lactose maltose, galactose, or sucrose, polysaccharides such as starch or cellulose or mixtures thereof and unpurified mixtures from renewable feedstocks such as cheese whey permeate, cornsteep liquor, sugar beet molasses, and barley malt. Other carbon substrates may include ethanol, lactate, succinate, or glycerol. Hence it is contemplated that the source of carbon in the media may encompass a wide variety of carbon containing substrates. Carbon substrates may also be provided by corn mash, cane juice, molasses, wheat mash, or other forms of biomass that have been liquefied, or treated and saccharified, to release carbon sources therein. Carbon substrates are typically maintained in excess to allow for maximal metabolism.

In addition to an appropriate carbon source, fermentation media must contain suitable minerals, salts, cofactors, buffers and other components, known to those skilled in the art, suitable for the metabolism of the cultures and promotion of the enzymatic pathway necessary for production of butanol.

Suitable media include common commercially prepared media such as broth that includes yeast nitrogen base, ammonium sulfate, and dextrose as the carbon/energy source or YPD Medium, a blend of peptone, yeast extract, and dextrose in optimal proportions for growing most Saccharomyces cerevisiae strains. Other defined or synthetic growth media may also be used and are known by one skilled in the art of microbiology or fermentation science.

### Culture Conditions

Typically cultures are maintained under conditions to support a viable butanol producing yeast cell, including a temperature in the range of about 20° C. to about 37° C. in an appropriate medium. Suitable pH ranges for the fermentation are typically between pH 3.0 and pH 7.5, where pH 4.5 to pH 6.5 is in some embodiments the initial condition.

Fermentations may be performed under aerobic or anaerobic conditions. In some embodiments, dissolved oxygen is maintained between microaerobic conditions to above 3%.

The amount of butanol in the fermentation medium is typically determined by high performance liquid chromatography (HPLC). However, other art-known methods can be used.

Cultures may be fermented in batch, fed-batch, or continuous systems. A Fed-Batch system is similar to a typical batch system with the exception that the carbon source substrate is added in increments as the fermentation progresses. Batch and Fed-Batch fermentations are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227, (1992), herein incorporated by reference.

Typical production conditions may include a fed-batch process for a period of time with a switch to a batch mode once all of the carbon source is added. Under commercial conditions, the liquefied mash or feedstock is fed over a period of time and a saccharification enzyme is also added to the fermentor which releases glucose from starch over time. This slow release of glucose over time from starch is controlled by the amount of saccharification enzyme that is added to the fermentor. In the case of cane juice fermentation, the substrate is slowly added over time until all substrates are added after which the fermentation proceeds under batch mode. The fermentation may be run for a period of time that is between about one hour and 200 hours.

Product Isolation from Fermentation Medium

During production, butanol product may be removed from the fermentation media by processes known in the art including vacuum application and liquid-liquid extraction.

Products can be isolated from the fermentation medium by methods known to one skilled in the art. For example, bioproduced isobutanol may be isolated from the fermentation medium using methods known in the art for ABE fermentations (see, e.g., Durre, Appl. Microbiol. Biotechnol. 49:639-648 (1998), Groot et al., Process. Biochem. 27:61-75 (1992), and references therein). For example, solids may be removed from the fermentation medium by centrifugation, filtration, decantation, or the like. Then, the isobutanol may be isolated from the fermentation medium using methods such as distillation, azeotropic distillation, liquid-liquid extraction, adsorption, gas stripping, membrane evaporation, pervaporation or vacuum flash fermentation (see e.g., U.S. Pub. No. 20090171129 A1, and International Pub. No. WO2010/151832 A1, both incorporated herein by reference in their entirety). A vacuum may be applied to a portion or the whole of the fermentation broth to remove butanol from the aqueous phase.

Because butanol forms a low boiling point, azeotropic mixture with water, distillation can be used to separate the mixture up to its azeotropic composition. Distillation may be used in combination with another separation method to obtain separation around the azeotrope. Methods that may be used in combination with distillation to isolate and purify butanol include, but are not limited to, decantation, liquid-liquid extraction, adsorption, and membrane-based techniques. Additionally, butanol may be isolated using azeotropic distillation using an entrainer (see, e.g., Doherty and Malone, Conceptual Design of Distillation Systems, McGraw Hill, New York, 2001).

The butanol-water mixture forms a heterogeneous azeotrope so that distillation may be used in combination with decantation to isolate and purify the isobutanol. In this method, the isobutanol containing fermentation broth is distilled to near the azeotropic composition. Then, the azeotropic mixture is condensed, and the isobutanol is separated from the fermentation medium by decantation. The decanted aqueous phase may be returned to the first distillation column as reflux. The isobutanol-rich decanted organic phase may be further purified by distillation in a second distillation column.

The butanol can also be isolated from the fermentation medium using liquid-liquid extraction in combination with distillation. In this method, the isobutanol is extracted from the fermentation broth using liquid-liquid extraction with a suitable solvent. The isobutanol-containing organic phase is then distilled to separate the butanol from the solvent. The amount of an extractant added may be from 5% to 50% of the fermentor volume for use in liquid-liquid extraction (LLE) to remove butanol from the aqueous medium during fermentation.

Distillation in combination with adsorption can also be used to isolate isobutanol from the fermentation medium. In this method, the fermentation broth containing the isobutanol is distilled to near the azeotropic composition and then the remaining water is removed by use of an adsorbent, such as molecular sieves (Aden et al., Lignocellulosic Biomass to Ethanol Process Design and Economics Utilizing Co-Current Dilute Acid Prehydrolysis and Enzymatic Hydrolysis for Corn Stover, Report NREL/TP-510-32438, National Renewable Energy Laboratory, June 2002).

Additionally, distillation in combination with pervaporation may be used to isolate and purify the isobutanol from the fermentation medium. In this method, the fermentation broth containing the isobutanol is distilled to near the azeotropic composition, and then the remaining water is removed by pervaporation through a hydrophilic membrane (Guo et al., J. Membr. Sci. 245, 199-210 (2004)).

In situ product removal (ISPR) (also referred to as extractive fermentation) can be used to remove butanol (or other fermentative alcohol) from the fermentation vessel as it is produced, thereby allowing the microorganism to produce butanol at high yields. One method for ISPR for removing fermentative alcohol that has been described in the art is liquid-liquid extraction. In general, with regard to butanol fermentation, for example, the fermentation medium, which includes the microorganism, is contacted with an organic extractant at a time before the butanol concentration reaches a toxic level. The organic extractant and the fermentation medium form a biphasic mixture. The butanol partitions into the organic extractant phase, decreasing the concentration in the aqueous phase containing the microorganism, thereby limiting the exposure of the microorganism to the inhibitory butanol.

Liquid-liquid extraction can be performed, for example, according to the processes described in U.S. Patent Appl. Pub. No. 20090305370, the disclosure of which is hereby incorporated in its entirety. U.S. Patent Appl. Pub. No. 20090305370 describes methods for producing and recovering butanol from a fermentation broth using liquid-liquid extraction, the methods comprising the step of contacting the fermentation broth with a water immiscible extractant to form a two-phase mixture comprising an aqueous phase and an organic phase. Typically, the extractant can be an organic extractant selected from the group consisting of saturated, mono-unsaturated, poly-unsaturated (and mixtures thereof) C₁₂ to C₂₂ fatty alcohols, C₁₂ to C₂₂ fatty acids, esters of C₁₂ to C₂₂ fatty acids, C₁₂ to C₂₂ fatty aldehydes, and mixtures thereof. The extractant(s) for ISPR can be non-alcohol extractants. The ISPR extractant can be an exogenous organic extractant such as oleyl alcohol, behenyl alcohol, cetyl alcohol, lauryl alcohol, myristyl alcohol, stearyl alcohol, 1-undecanol, oleic acid, lauric acid, myristic acid, stearic acid, methyl myristate, methyl oleate, undecanal, lauric aldehyde, 20-methylundecanal, and mixtures thereof.

In some embodiments, the alcohol can be esterfied by contacting the alcohol in a fermentation medium with an organic acid (e.g., fatty acids) and a catalyst (e.g. enzyme such as a lipase) capable of esterfiying the alcohol with the organic acid. In such embodiments, the organic acid can serve as an ISPR extractant into which the alcohol esters partition. The organic acid can be supplied to the fermentation vessel and/or derived from the biomass supplying fermentable carbon fed to the fermentation vessel. Lipids present in the feedstock can be catalytically hydrolyzed to organic acid, and the same catalyst (e.g., enzymes) can esterify the organic acid with the alcohol. The catalyst can be supplied to the feedstock prior to fermentation, or can be supplied to the fermentation vessel before or contemporaneously with the supplying of the feedstock. When the catalyst is supplied to the fermentation vessel, alcohol esters can be obtained by hydrolysis of the lipids into organic acid and substantially simultaneous esterification of the organic acid with butanol present in the fermentation vessel. Organic acid and/or native oil not derived from the feedstock can also be fed to the fermentation vessel, with the native oil being hydrolyzed into organic acid. Any organic acid not esterified with the alcohol can serve as part of the ISPR extractant. The extractant containing alcohol esters can be separated from the fermentation medium, and the alcohol can be recovered from the extractant. The extractant can be recycled to the fermentation vessel. Thus, in the case of butanol production, for example, the conversion of the butanol to an ester reduces the free butanol concentration in the fermentation medium, shielding the microorganism from the toxic effect of increasing butanol concentration. In addition, unfractionated grain can be used as feedstock without separation of lipids therein, since the lipids can be catalytically hydrolyzed to organic acid, thereby decreasing the rate of build-up of lipids in the ISPR extractant.

In situ product removal can be carried out in a batch mode or a continuous mode. In a continuous mode of in situ product removal, product is continually removed from the reactor. In a batchwise mode of in situ product removal, a volume of organic extractant is added to the fermentation vessel and the extractant is not removed during the process. For in situ product removal, the organic extractant can contact the fermentation medium at the start of the fermentation forming a biphasic fermentation medium. Alternatively, the organic extractant can contact the fermentation medium after the microorganism has achieved a desired amount of growth, which can be determined by measuring the optical density of the culture. Further, the organic extractant can contact the fermentation medium at a time at which the product alcohol level in the fermentation medium reaches a preselected level. In the case of butanol production according to some embodiments of the present invention, the organic acid extractant can contact the fermentation medium at a time before the butanol concentration reaches a toxic level, so as to esterify the butanol with the organic acid to produce butanol esters and consequently reduce the concentration of butanol in the fermentation vessel. The ester-containing organic phase can then be removed from the fermentation vessel (and separated from the fermentation broth which constitutes the aqueous phase) after a desired effective titer of the butanol esters is achieved. In some embodiments, the ester-containing organic phase is separated from the aqueous phase after fermentation of the available fermentable sugar in the fermentation vessel is substantially complete.

### EXAMPLES

The present invention is further defined in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “OD₆₀₀” means the optical density measured at a wavelength of 600 nm, “CFU” means colony forming unit, “HPLC” means high performance liquid chromatography.

### GENERAL METHODS

Media and growth of yeast are described in Amberg, Burke and Strathern, 2005, Methods in Yeast Genetics, Cold Spring Harbor Laboratory Press, Cold Spring Harbor, N.Y. Methods are also described in Yeast Protocol (Editor W. Xiao, Humana Press, Totowa, N.J. The optical density at 600 nm (OD₆₀₀) was measured using an Ultraspec 3100 spectrophotometer (GE Healthcare Life Sciences, Piscataway, N.J.).

### Example 1

### 24 Hour Survival at Low Cell Density and High Cell Density for Saccharomyces cerevisiae BY4743

Saccharomyces cerevisiae BY4743 (ATCC 201390) was plated from a freezer vial onto YPD agar (Teknova, Hollister, Calif.; Cat. #Y1000) and incubated overnight at 30° C. Cells from the YPD plate were inoculated into a 25 ml pre-culture of YPD broth (Teknova; Cat. #Y5000) with an initial OD₆₀₀ of 0.06 to 0.08. The pre-culture was grown for 7 hours in an aerobic shaker at 30° C. An aliquot of the pre-culture was inoculated into 200 ml of YPD broth, with an initial OD₆₀₀ of 0.06 to 0.08, and this culture was incubated with aeration at 30° C. for approximately 17 hours. The optical density of the culture was measured, the culture was centrifuged and the cells were resuspended to yield an OD₆₀₀ of 5 in fresh YPD broth. This is referred to as a high cell density (HCD) culture. This culture was diluted 100 fold into fresh YPD broth to give a culture with an OD₆₀₀ of 0.05. This is referred to as a low the cell density (LCD) culture. These cultures were incubated at 30° C. with shaking for 30 minutes for an acclimation period. At the end of the 30 minute acclimation period, the OD₆₀₀ was measured. 5 ml of low or high cell density culture was dispensed to 15 ml conical tubes which contained different concentrations of the alcohol to be tested. A control tube included no alcohol. The tubes were placed in a rotary drum at 30° C. and incubated for 24 hours. The time of addition of cells was referred to as “time 0”. The concentrations tested were: 1-butanol at 1.0% (w/v), 1.5% and 2%; isobutanol at 1.0%, 1.5% and 2.5%; 2-butanol at 2.0%, 3.0% and 3.5%; and ethanol at 2.0%, 4.0%, and 5%. The alcohol concentration of each culture was measured after 24 hours by HPLC which showed that <0.01% of the alcohol was lost during the incubation period.

Colony forming units (CFUs) were determined on YPD plates at time 0 for the control and at 24 hours for each of the cultures, including the control culture, using standard microbiological methods. Cultures were serially diluted in a microtiter plate (20 μl of the culture and 180 μl of YPD broth) and 10 μl of the various dilutions were spotted onto triplicate agar plates and incubated at 30° C. for 24 and 48 hours. Colonies were counted after 48 hours, and CFUs/ml were calculated. At time 0, the low cell density cultures had about 4.8×10⁵ CFUs/ml, and the high cell density cultures had about 5.4×10⁷ CFUs/ml. The percent survival was calculated based on the CFUs/ml of the 24 hour control (0 butanol) HCD or LCD culture, and the results are given in Table 2.

TABLE 2
Survival of S. cerevisiae after 24 h exposure to variousalcohols

| | Concentration | Percent Survival | |
|---|---|---|---|
| Alcohol | (w/v) | HCD (%) | LCD (%) |
| 1-Butanol | 1.0% | 72.2 | 1.3 |
| | 1.5% | 57.3 | 0.4 |
| | 2.0% | 3.6 | 0.1 |
| Isobutanol | 1% | 77.8 | 0.6 |
| | 1.5% | 45.1 | 0.1 |
| | 2.5% | <0.001 | <0.001 |
| 2-Butanol | 2% | 27.6 | 0.3 |
| | 3% | 18.9 | 0.1 |
| | 3.5% | <0.001 | <0.001 |
| Ethanol | 2% | 62.5 | 100.0 |
| | 4% | 50.2 | 100.0 |
| | 5% | 38.9 | 25.3 |

The control high cell density culture without alcohol increased from 5.4×10⁷ cells/ml to 6.5×10⁷ cells/ml in 24 hours. The control low cell density culture without alcohol increased from 4.8×10⁵ cells/ml to 2.8×10⁷ cells/ml in 24 hours. In cultures exposed to 1-butanol, isobutanol, or 2-butanol, the percent survival was significantly higher for the high cell density cultures than for the low cell density cultures (Table 2). In contrast, high cell density cultures were more sensitive to exposure to 2.0% or 4.0% ethanol than the low cell density cultures.

### Example 2

### 24 Hour Survival at Low Cell Density and High Cell Density for Non-Saccharomyces Yeasts

Non-Saccharomyces yeast strains were tested to determine whether isobutanol tolerance is affected by cell density for these yeasts. Kluyveromyces marxianus PNY0577 (American Type Culture Collection (“ATCC”), Manassas, Va.; ATCC #8554), Kluyveromyces marxians PNY0578 (ATCC #16045), Pichia membranifaciens PNY0572, Pichia anomala PNY0573, Pichia sp. PNY0574, Issatchenkia orientalis PNY0575 and Issatchenkia orientalis PNY0576 were tested. The Pichia and Issatchenkia strains used are wild-type representatives of the designated genera. Other representative strains are commercially available, for example, from ATCC.

The non-Saccharomyces yeast strains were plated from freezer vials onto YPD agar (Teknova, Hollister, Calif.) and incubated overnight at 30° C. Cells from each strain from the YPD plates were inoculated into a 25 ml pre-culture of YPD broth) (Teknova, Hollister, Calif.) with an initial OD₆₀₀ of 0.06 to 0.08 and grown for 7 hours in an aerobic shaker at 30° C. An aliquot of each pre-culture was inoculated into 200 ml YPD broth, to provide an initial OD₆₀₀ of 0.06 to 0.08 and the resulting culture was incubated with aeration at 30° C. for approximately 17 hours. The optical density of the culture was measured, the culture was centrifuged and the cells were resuspended to yield an OD₆₀₀ of 5 in fresh YPD broth. This is referred to as a high cell density (HCD) culture. This culture was diluted 100 fold into fresh YPD broth to give a culture with an OD₆₀₀ of 0.05. This is referred to as a low cell density (LCD) culture. These cultures were incubated at 30° C. with shaking for 30 minutes for an acclimation period. At the end of the 30 minute acclimation period, the OD₆₀₀ was measured.

5 ml of low or high cell density culture was dispensed to 15 ml conical tubes which contained the concentrations of isobutanol to be tested. The tubes were placed in a rotary drum at 30° C. and incubated for 24 hours. The isobutanol concentration was 1.0% or 2.0%. A control culture for each strain contained no isobutanol. The time of addition of cells was “time 0”. The isobutanol concentration of each sample was measured after 24 hours by HPLC which showed that <0.01% of the alcohol was lost during the incubation period. All samples were filtered with Acrodisc CR PTFE 0.2 μm filters (Pall Life Sciences, Port Washington, N.Y.) and analyzed using a Shodex SH1011 column (8 mm ID×300 mm length; Showa Denko America, Inc., New York, N.Y.) and a Shodex SH-G as a guard column. The injection volume was 10 μL. The mobile phase was 0.01N sulfuric acid. The column temperature was 50° C. with a mobile phase flow rate of 0.5 mL/min. For detection, a photometric detector at 210 nm and a refractive index detector were used.

Colony forming units (CFUs) were determined on YPD plates at time 0 for the controls and at 24 hours for all samples, including the control cultures, using standard microbiological methods. Essentially, cells were serially diluted in a microtiter plate (20 μl of the culture and 180 μl of YPD broth) and 10 μl of the various dilutions were spotted onto triplicate agar plates and incubated at 30° C. for 24 and 48 hours. Colonies were counted after 48 hours and CFUs/ml were calculated. The percent survival was calculated based on the CFUs/ml of the 24 hour HCD or LCD control culture for each strain and results are given in Table 3.

The control high cell density cultures without isobutanol increased from about 1.0×10⁸ cells/ml to about 1.5×10⁸ cells/ml in 24 hours. The control low cell density cultures without isobutanol increased from about 1.1×10⁶ cells/ml to about 1.0×10⁸ cells/ml in 24 hours. All of the non-Saccharomyces yeast strains had significantly higher levels of survival in HCD cultures than in LCD cultures exposed to 1.0% isobutanol (Table 3). Only one strain (PNY0574) survived exposure to 2.0% isobutanol. This strain also had a significantly higher level of survival in the HCD culture than in the LCD culture exposed to 2.0% isobutanol.

TABLE 3
Survival of non-Saccharomyces yeasts after 24 h exposure to isobutanol

| | | | | Percent Survival | |
|---|---|---|---|---|---|
| | | | | 1% isobutanol | 2% isobutanol |
| Strain ID | Name | HCD | LCD | HCD | LCD |
| PNY0572 | Pichia membranifaciens | 80.6 | 2.7 | <0.001 | <0.001 |
| PNY0573 | Pichia anomala | 75.6 | 0.1 | <0.001 | <0.001 |
| PNY0574 | Pichia sp. | 100 | 10 | 100 | 0.5 |
| PNY0575 | Issatchenkia orientalis | 61.3 | 3.1 | <0.001 | <0.001 |
| PNY0576 | Issatchenkia orientalis | 50 | 12.8 | <0.001 | <0.001 |
| PNY0577 | Kluyveromyces marxianus | 39.5 | 0.1 | <0.001 | <0.001 |
| PNY0578 | Kluyveromyces marxianus | 51 | <0.001 | <0.001 | <0.001 |

### Example 3

Glucose Utilization Rates for Saccharomyces cerevisiae Strains in the Presence of Isobutanol in High Cell Density Culture

Saccharomyces cerevisiae BY4743 was plated from a freezer vial onto YPD agar (Teknova, Hollister, Calif.; Cat. #Y1000) and incubated overnight at 30° C. Cells from the YPD plate were inoculated into a 25 ml pre-culture of YPD broth (Teknova, Hollister, Calif.; Cat. #Y5000) with an initial OD₆₀₀ of 0.06 to 0.08, and this culture was grown for 7 hours in an air shaker at 30° C. An aliquot of the pre-culture was inoculated into 200 ml of YPD broth with an initial OD₆₀₀ of 0.06 to 0.08, and this culture was incubated with aeration at 30° C. for approximately 17 hours. The optical density of the culture was determined, the culture was centrifuged and the cells were resuspended in fresh YPD broth to yield an OD₆₀₀ of 8. At this point, an aliquot of 10 ml of the culture was used for a dry cell weight determination, which gave a result of 3.89 gdcw/L.

20 ml cultures were transferred to flasks that contained 0.0%, 0.5%, 1.0%, 1.25%, 1.5%, 1.75% or 2.0% isobutanol. Time of addition of cells was referred to as 0 hours. At various times, samples (1.5 ml) were withdrawn and centrifuged at 10,000 rpm for 2 minutes in a microfuge (Sorvall Biofuge pico). The supernatant was filtered through a 0.2 μm filter (Pall Life Sciences, Port Washington, N.Y.) Pall GHP Acrodisc 13 mm Syringe Filter with 0.2 μm GHP Membrane), the filtrate was diluted ten-fold and the glucose concentration was determined using a YSI Glucose Analyzer (YSI 2700 Select; YSI, Inc., Yellow Springs, Ohio).

Glucose consumption rates in the presence of isobutanol were also determined when the cell concentration was 18 gdcw/L or 24 gdcw/L. Cells from the YPD plate were inoculated into a 25 ml pre-culture of YPD broth (Teknova, Hollister, Calif. Cat. #Y5000) with an initial OD₆₀₀ of 0.06 to 0.08, and this culture was grown for 7 hours in an air shaker at 30° C. An aliquot of the pre-culture was inoculated into 8 flasks with 300 ml of YPD broth with an initial OD₆₀₀ of 0.1 and this culture was incubated with aeration at 30° C. for approximately 17 hours. The optical density of the culture was determined, the culture was centrifuged and the cells were resuspended in fresh YPD broth to yield an OD₆₀₀ of 38 (18 gdcw/L). Samples (15 ml) were transferred to flasks containing 2%, 3%, or 4% isobutanol and glucose consumption was followed for 150 min.

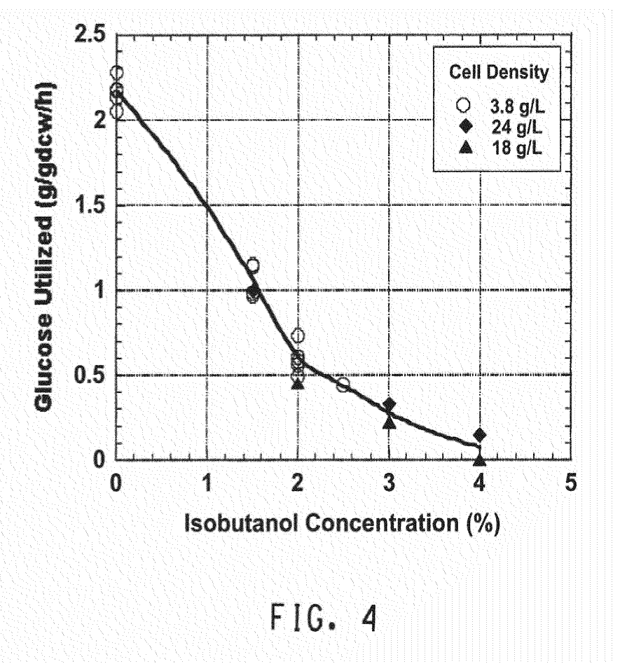

The experiment was repeated except that the starting OD₆₀₀ was 49.1 (24 gdcw/L). Samples (15 ml) were transferred to flasks containing 1.5%, 3%, or 4% isobutanol and glucose consumption was followed for 150 min. The results for BY4743 are graphed in FIG. 4.

In the control culture (without isobutanol) the glucose consumption rate was around 2.17 g/gdcw/h under the experimental conditions tested. At 1.5% isobutanol the glucose consumption rate for both the 3.8 and the 24 gdcw/L cultures was approximately 50% of the control rate (about 1.1 g/gdcw/h). Glucose consumption was observed even in the presence of 2.0% and 2.5% isobutanol, and it ranged from 20 to 25% of the control rate. The actual glucose consumption rate in the control may vary 5% from one experiment to another. However, the relative percentage of isobutanol inhibition were reproducible.

### Example 4

### Process to Generate High Cell Density Culture

Saccharomyces cerevisiae BY4743 was plated from a freezer vial onto YPD agar (Teknova, Hollister, Calif.; Cat. #Y1000) and incubated overnight at 30° C. Cells from the YPD plate were inoculated into a 25 ml pre-culture of YPD broth (Teknova, Hollister, Calif.; Cat. #Y5000) with an initial OD₆₀₀ of 0.06 to 0.08. The pre-culture was grown for 7 hours in an aerobic shaker at 30° C. An aliquot of the pre-culture was inoculated into 125 ml of YPD broth, with an initial OD₆₀₀ of 0.06 to 0.08, and this culture was incubated with aeration at 30° C. for approximately 17 hours.

A starter culture of 150 ml of YPD was inoculated with 5 ml of the overnight culture with an initial OD₆₀₀ of 0.15. This culture was incubated at 30° C. with aeration for two hours, reaching OD₆₀₀ of 0.38. At this time (time=0) the cultures were challenged with varying concentrations of isobutanol as given in Table 4 and the OD₆₀₀ was assayed every hour for 5 h and at 24 h. Results are given in Table 4.

TABLE 4
Growth (OD 600) of yeast in varying isobutanol concentrations

| | BY4743 | | | | | | |
|---|---|---|---|---|---|---|---|
| Time | Isobutanol Concentration | | | | | | |
| (Hours) | 0% | 0.5% | 0.75% | 1% | 1.5% | 2% | 2.5% |
| 0 | 0.38 | 0.38 | 0.38 | 0.38 | 0.38 | 0.38 | 0.38 |
| 1 | 0.56 | 0.55 | 0.54 | 0.51 | 0.46 | 0.43 | 0.42 |
| 2 | 0.80 | 0.74 | 0.68 | 0.62 | 0.50 | 0.46 | 0.42 |
| 3 | 1.42 | 1.12 | 1.00 | 0.82 | 0.53 | 0.47 | 0.42 |
| 4 | 2.32 | 1.69 | 1.36 | 0.96 | 0.61 | 0.49 | 0.45 |
| 5 | 3.02 | 2.22 | 1.80 | 1.16 | 0.65 | 0.50 | 0.45 |
| 24 | 6.44 | 5.19 | 4.97 | 4.41 | 1.41 | 0.90 | 0.52 |

Since for this strain, one OD₆₀₀ is equivalent to 10⁷ cells/ml, S. cerevisiae grew to about 4×10⁷ cells/ml in the presence of 1% isobutanol after overnight growth.

The cultures grown in 1% isobutanol, or less (concentration of isobutanol <1%), to OD₆₀₀ of 4.41, or greater, are high cell density cultures. These cultures can be used for isobutanol production.

### Example 5

### Process to Generate Strains with High Glucose Consumption

S. cerevisiae strains used were PNY0569 CEN.PK122 (MATa MAL2-8c SUC2/MATalpha MAL2-8c SUC2) and PNY0571 (CEN.PK 113-7D MATa MAL2-8c SUC2), obtained from Centraalbureau voor Schimmelcultures, Fungal and Yeast Collection (Netherlands) as well as PNY0602 and PNY0614. PNY0602 and PNY0614 were isolated from a mutagenized culture of PNY0571. PNY0571 (CEN.PK 113-7D) was subjected to 10 rounds of chemical mutagenesis with N-methyl-N′-nitro-N-nitrosoguanidine (NTG) and ethylmethane sulfonate (EMS) using standard methods (Barbour, L., M. Hanna and W. Xiao. 2006. Mutagenesis, p. 121-127. In W. Xiao (ed.), Yeast Protocols, Second Edition. Humana Press, NJ). Cells were grown overnight in 10 ml of YPD at 30° C. with shaking. The overnight culture was centrifuged, and the pellet was resuspended in 50 mM potassium phosphate buffer, pH 7.0. The cells were again centrifuged and resuspended in 10 ml of the same buffer. A portion of the resuspended cells (2.5 ml) was transferred to a plastic 15 ml screw cap centrifuge tube, and the cells were treated with NTG (10 μg/ml final concentration) or EMS (3% w/v) for 40 minutes at 30° C. without shaking. The mutagen was inactivated by addition of an equal volume of filter sterilized 10% (w/v) sodium thiosulfate. The treated cells were centrifuged and resuspended in water two times. The treatment protocol involved repeated cycles of treating yeast cells with one of the mutagens (e.g., NTG), allowing the surviving cells to grow out overnight in YPD with 1% isobutanol, and then treating the overnight culture with the other mutagen (e.g., EMS). After the fifth cycle (i.e., a total of ten treatments with mutagen), cells were screened for isobutanol tolerance. PNY0602 was isolated after prolonged (24 h) exposure to 3.0% isobutanol. PNY0614 was isolated after 5 cycles of repeated freezing and thawing of the mutagenized culture by resuspending mutagenized cells in distilled water and transferring the cells to dry-ice ethanol bath and a 37° C. water bath for 20 minutes each.

The isolated microorganism associated with ATCC Accession No. ______ is also known herein as PNY0602. The isolated microorganism associated with ATCC Accession No. ______ was deposited under the Budapest Treaty on ______ at the American Type Culture Collection, Patent Depository 10801 University Boulevard, Manassas, Va. 20110-2209. The isolated microorganism associated with ATCC Accession No. ______ is also known herein as PNY0614. The isolated microorganism associated with ATCC Accession No. ______ was deposited under the Budapest Treaty on ______ at the American Type Culture Collection, Patent Depository 10801 University Boulevard, Manassas, Va. 20110-2209.

Glucose consumption rates in the presence of isobutanol in YPD were also determined when the cell concentration was about 8 O.D. (about 3.9 gdcw/L). Saccharomyces cerevisiae strains PNY0571, PNY0602 and PNY0614 were plated from a freezer vial onto YPD agar (Teknova, Hollister, Calif.; Cat. #Y1000) and incubated overnight at 30° C. Cells from the YPD plate were inoculated into a 25 ml pre-culture of YPD broth (Teknova, Hollister, Calif.; Cat. #Y5000) with an initial OD₆₀₀ of 0.06 to 0.08, and this culture was grown for 7 hours in an air shaker at 30° C. An aliquot of the pre-culture was inoculated into 200 ml of YPD broth with an initial OD₆₀₀ of 0.06 to 0.08, and this culture was incubated with aeration at 30° C. for approximately 17 hours. The optical density of the culture was determined, the culture was centrifuged and the cells were resuspended in fresh YPD broth to yield an OD₆₀₀ of 8. At this point, an aliquot of 10 ml of the culture was used for a dry cell weight determination, which gave dry cell weight in the range of 3.89 gdcw/L.

20 ml cultures were transferred to flasks that contained 0.0%, 0.5%, 0.75%, 1.0%, 1.25%, 1.5%, 1.75%, 2.0% and 2.5% isobutanol. Time of addition of cells was referred to as 0 hours. At various times samples (500 to 700 ul) were transferred to a tube containing an equal volume of 10% TCA. Samples were centrifuged and glucose determined in the YSI glucose analyzer (YSI 2700 Select).

Results are shown in Table 5. These results show that the glucose consumption rates can be improved compared with the parent by mutagenesis and selection.

TABLE 5
Glucose consumed per gdcw per hour in high cell density
cultures with differing amounts of isobutanol.

| | Isobutanol Concentration (%) | | | | | | | |
|---|---|---|---|---|---|---|---|---|
| Strain | 0 | 0.5 | 0.75 | 1 | 1.5 | 1.75 | 2 | 2.5 |
| PNY0571 | 2.4 | 2.1 | 2.0 | 1.9 | 1.0 | 0.8 | 0.6 | 0.3 |
| PNY0602 | 3.1 | 3.0 | 2.9 | 2.7 | 1.5 | 1.1 | 0.9 | 0.5 |
| PNY0614 | 3.0 | 2.9 | 2.8 | 2.6 | 1.4 | 1.0 | 0.8 | 0.4 |

Isobutanol production may or may not be coupled to growth. Therefore we measured the glucose consumption rates under non-growing conditions using three different pH buffers.

Glucose consumption rates were determined in phosphate buffer pH 6.5 as outlined by Diderich, J. A., et al., Microbiology, 1999, 145 p. 3447-54. We also determined the glucose consumption rates at pH 5.25 and pH 4.0 using MES buffer.

Cells were grown overnight at 30° C. in YPD (125 ml in a 500 ml flask). Cells were centrifuged and resuspended in buffer (0.1M phosphate buffer pH 6.0 or 0.1M MES buffer pH 5.25 or 0.1M MES buffer pH 4.0), washed one time, and then resuspended such that each cell suspension had approximately 16 OD or 160 million cell/ml. 10 ml of these stock cells were transferred to flasks containing 10 ml of the corresponding buffer containing 40 g/L glucose and varying concentrations of isobutanol. At various times samples (500 to 700 ul) were transferred to a tube containing an equal volume of 10% TCA. Samples were centrifuged and glucose determined in the YSI glucose analyzer. Glucose consumption rates were determined by plotting the amount of glucose consumed over time. The results are given in Table 6. Higher glucose consumption rates were observed in the presence of 20 g/L isobutanol at lower pHs than at pH 6.5.

TABLE 6
Glucose utilization rates (g/gdcw/h) in the presence of 20 g/L isobutanol,
at three different pH values.

| 20 g/L | Glucose Utilization (g/gdcw/h) | | |
|---|---|---|---|
| Isobutanol | pH 4 | pH 5.25 | pH 6.5 |
| PNY0569 | 0.7 | 0.8 | 0.6 |
| PNY0602 | 0.9 | 1.1 | 0.7 |
| PNY0614 | 0.9 | 1.1 | 0.7 |

### Example 6

### Glucose Utilization Rates for Issatchenkia orientalis Strains in the Presence of Isobutanol in High Cell Density Culture

Strain Issatchenkia orientalis PNY0660, was derived from ATCC 20381 strain that was previously referred to as Candida acidothermophilium. Strain PNY0660 was plated from a freezer vial onto YPD agar (Teknova, Hollister, Calif.; Cat. #Y1000) and incubated overnight at 30° C. Cells from the YPD plate were inoculated into a 50 ml pre-culture of YPD broth (Teknova, Hollister, Calif.; Cat. #Y5000) with an initial OD₆₀₀ of 0.06 to 0.08, and this culture was grown for 5 hours in an air shaker at 30° C. An aliquot of the pre-culture was inoculated into 200 ml of YPD broth with an initial OD₆₀₀ of 0.6, and this culture was incubated with aeration at 30° C. for approximately 17 hours. The optical density of the culture was determined, the culture was centrifuged, and the sugar consumption rate was determined in corn test medium. Corn test medium contained 0.2% casamino acids and 2% glucose and 100 mM MES buffer pH 5.25 and per liter contained (i) salts: ammonium sulfate 5.0 g, potassium phosphate monobasic 2.8 g, and magnesium sulfate heptahydrate 0.5 g, (ii) vitamins: biotin (D−) 0.40 mg, Ca D(+) panthotenate 8.00 mg, myo-inositol 200.00 mg, pyridoxol hydrochloride 8.00 mg, p-aminobenzoic acid 1.60 mg, riboflavin 1.60 mg, folic acid 0.02 mg, niacin 30.0 mg, and thiamine 30 mg; and (iii) trace elements: EDTA (Titriplex 1117) 99.38 mg, zinc sulphate heptahydrate 29.81 mg, manganese chloride dehydrate 5.57 mg, cobalt(II)chloride hexahydrate 1.99 mg, copper(II)sulphate pentahydrate 1.99 mg, Di-sodium molybdenum dehydrate 2.65 mg, calcium chloride dehydrate 29.81 mg, iron sulphate heptahydrate 19.88 mg, and boric acid.

20 ml cultures (2.7 gdcw/L) in corn test medium were transferred to flasks that contained 0.0%, 0.5%, 1.0%, 1.5%, 1.75% or 2.0% isobutanol. Time of addition of cells was referred to as 0 hours. At various times, samples (1.5 ml) were withdrawn and centrifuged at 10,000 rpm for 2 minutes in a microfuge (Sorvall Biofuge pico). The supernatant was filtered through a 0.2 μm filter (Pall Life Sciences, Port Washington, N.Y.) Pall GHP Acrodisc 13 mm Syringe Filter with 0.2 μm GHP Membrane), the filtrate was diluted ten-fold, and the glucose concentration was determined using a YSI Glucose Analyzer (YSI 2700 Select; YSI, Inc., Yellow Springs, Ohio). The sugar consumption rate that was measured from 0 h to 6 h is shown in Table 7.

TABLE 7
Glucose utilization rate g/gdcw/h

| Isobutanol (g/L) | 0.0 | 5.0 | 10.0 | 15.0 | 17.5 | 20.0 |
|---|---|---|---|---|---|---|
| 30 C. | 2.4 | 2.4 | 2.4 | 1.4 | 0.9 | 0.7 |

I. orientalis strains have a sugar consumption rate of 0.7 g/gdw/h in the presence of 20 g/l of isobutanol.

The foregoing description of the invention has been presented for purposes of illustration and description. Furthermore, the description is not intended to limit the invention to the form disclosed herein.

All of the various aspects, embodiments and options described herein can be combined in any and all variations.

All publications, patents and patent applications mentioned in this specification are herein incorporated by reference to the same extent as if each individual publication, patent or patent application was specifically and individually indicated to be incorporated by reference.

What is claimed is:

1. A production culture for the fermentative production of butanol comprising:
(i) a medium comprising a fermentable carbon substrate;
(ii) a culture of yeast cells having a glucose utilization rate of at least about 0.5 gram per gram of dry cell weight per hour; and
(iii) butanol at a concentration of at least about 2% (w/v) in the medium.

2. The production culture of claim 1, wherein the cell density is at least about 2.4 gram dry cell weight per liter.

3. The production culture of claim 2, wherein the cell density is at least about 7 gram dry cell weight per liter.

4. The production culture of claim 1, wherein the butanol producing yeast cells have a glucose utilization rate of at least about 1 gram per gram of dry cell weight per hour.

5. The production culture of claim 4, wherein the butanol producing yeast cells are produced by a method comprising:
(i) mutagenesis;
(ii) exposure to 3% isobutanol; and
(iii) repeated freeze-thaw cycles.

6. The production culture of claim 1, wherein the yeast is crabtree positive.

7. The production culture of claim 1, wherein the yeast is a member of a genus selected from the group consisting of Saccharomyces, Schizosaccharomyces, Hansenula, Candida, Kluyveromyces, Yarrowia, Issatchenkia and Pichia.

8. The production culture of claim 1, wherein the yeast comprises an isobutanol pathway or a 1-butanol pathway.

9. The production culture of claim 1, wherein the culture is maintained at the following conditions for about 1 hour to about 200 hours:
(i) temperature that is from about 20° C. to about 45° C.;
(ii) dissolved oxygen that is maintained at microaerobic conditions to above 3%;
(iii) carbon substrates in excess provided by liquefied biomass;
(iv) pH that is from about 3 to about 7.5; and
(v) butanol removal selected from vacuum application and liquid-liquid extraction.

10. A method for the production of butanol, comprising:
(i) preparing the production culture of claim 1, wherein the yeast comprises an isobutanol pathway or a 1-butanol pathway; and
(ii) fermenting the yeast under conditions wherein butanol is produced.

11. A production culture for the fermentative production of butanol comprising:
(i) a medium comprising a fermentable carbon substrate;
(ii) a culture of yeast cells having a cell density of at least about 2.4 gram dry cell weight per liter; and
(iii) butanol at a concentration of at least about 2% (w/v) in the medium.

12. The production culture of claim 11, wherein the culture has a glucose utilization rate of at least about 0.5 gram per gram of dry cell weight per hour.

13. The production culture of claim 11, wherein the culture has a glucose utilization rate of at least about 1 gram per gram of dry cell weight per hour

14. The production culture of claim 11, wherein the cell density is at least about 7 grams dry cell weight per liter.

15. The production culture of claim 11, wherein the butanol concentration is at least about 2.5% and the culture has a glucose utilization rate of at least about 0.4 gram per gram of dry cell weight per hour.

16. The production culture of claim 11, wherein the yeast is a member of a genus selected from the group consisting of Saccharomyces, Schizosaccharomyces, Hansenula, Candida, Kluyveromyces, Yarrowia, Issatchenkia and Pichia.

17. The production culture of claim 11, wherein the yeast comprises an isobutanol pathway or 1-butanol pathway.

18. The production culture of claim 11, wherein the culture is maintained at the following conditions for about 1 hour to about 200 hours:
(i) temperature that is from about 20° C. to about 37° C.;
(ii) dissolved oxygen that is maintained at microaerobic conditions to above 3%;
(iii) carbon substrates in excess provided by liquefied biomass;
(iv) pH that is from about 3 to about 7.5; and
(v) butanol removal selected from vacuum application and liquid-liquid extraction.

19. A method for the production of butanol, comprising:
a. preparing the production culture of claim 11, wherein the yeast comprises a butanol biosynthetic pathway selected from the group consisting of an isobutanol pathway, a 1-butanol pathway and a 2-butanol pathway; and
b. fermenting the yeast under conditions wherein butanol is produced.

20. A method for increasing the tolerance of a production culture for the fermentative production of butanol, comprising:
(i) providing a medium comprising a fermentable carbon substrate;
(ii) providing a culture of butanol producing yeast cells having a glucose utilization rate of at least about 0.5 gram per gram of dry cell weight per hour; and
(iii) contacting the yeast culture with the fermentable carbon substrate whereby the glucose utilization rate is maintained over a suitable period of time and whereby butanol is produced.

21. A production culture for the fermentative production of butanol comprising:
(i) a medium comprising a fermentable carbon substrate;
(ii) a culture of yeast cells having a glucose utilization rate of at least about 2.4 grams per gram of dry cell weight per hour; and
(iii) butanol at a concentration of at least about 1% (w/v) in the medium.

22. The production culture of claim 21, wherein the cell density is at least about 2.7 grams dry cell weight per liter.
